(19) United States
(12) Patent Application Publication
(10) Pub. No.: US 2023/0360730 A1
(43) Pub. Date: Nov. 9, 2023

(54) SYSTEMS AND METHODS FOR ANALYSIS OF SAMPLES

(71) Applicant: IDbyDNA Inc., Salt Lake City, UT (US)

(72) Inventors: Kate Broadbent, Salt Lake City, UT (US); Robert Schlaberg, Salt Lake City, UT (US)

(21) Appl. No.: 18/003,648

(22) PCT Filed: Feb. 4, 2022
(86) PCT No.: PCT/US2022/015355

§ 371 Date: Dec. 28, 2022

Related U.S. Application Data
(60) Provisional application No. 63/145,954, filed on Feb. 4, 2021.

(51) Int. Cl. G16B 30/10; G16B 25/10

(52) U.S. Cl. CPC G16B 30/10; G16B 25/10

(57) ABSTRACT

Systems and methods for determining an amount of a predefined category are provided. A sample is obtained, including nucleic acids from the predefined category and nucleic acids from a source other than the predefined category. A known quantity of an internal control material comprising nucleic acids is added to the sample. The sample, including the internal control material, is sequenced. A sequencing dataset including sequence reads from the predefined category and sequence reads from the internal control material is obtained. A first read count, normalized using a first target nucleotide length, of sequence reads from the predefined category, and a second read count, normalized using a second target nucleotide length, of sequence reads from the internal control material are determined. The amount of the predefined category in the sample is calculated based on the first read count, the second read count, and the known quantity of the internal control material.

### CROSS REFERENCE TO RELATED APPLICATIONS

This application claims priority to U.S. Provisional Patent Application Ser. No. 63/145,954, filed Feb. 4, 2021, which is hereby incorporated by reference in its entirety.

### TECHNICAL FIELD

This specification describes technologies relating to quantifying predefined categories, such as organisms, represented within a sample.

### BACKGROUND

The paradigm of DNA sequencing has changed with the advent of next-generation sequencing (NGS) technologies capable of processing hundreds of thousands to millions of DNA fragments in parallel, resulting in low per-base costs for generated sequences and gigabase (Gb) to terabase (Tb)-scale throughputs for a single sequencing run. A modern NGS sequencer, for example, can sequence over 45 human genomes in a single day for approximately $1000 each, or less. Consequently, NGS can be used to define the characteristics of entire genomes and delineate differences between them, allowing researchers to gain a deeper understanding of the full spectrum of genetic variation underlying complex phenotypic traits. Wide availability of next-generation sequencing instruments, lower reagent costs, and streamlined sample preparation protocols are enabling an increasing number of investigators to perform rapid, cost-effective, and high-throughput DNA and RNA sequencing for metagenomics studies. These approaches reduce bias, improve detection of less abundant taxa, and facilitate the discovery of novel pathogens and pathogenic markers.

Nevertheless, NGS protocols are highly complex and variable, giving rise to intra- or inter-lab variation magnified over differences in, for example, starting sample, reagents, instruments, library preparation, sequencing, and/or other avenues for sample loss or human error. Such variation limits the clinical and diagnostic value of NGS data, for instance, where meaningful analysis of sequencing data from multiple sources is hindered by inconsistencies between samples, sequencing runs, batches, or labs. In particular, sample-to-sample or lab-to-lab variations can prevent the accurate comparison, quantification, or determination of prevalence of populations (e.g., organismal populations) in samples for use in clinical and molecular diagnostics.

### SUMMARY

Given the above background, improved methods and systems are needed for performing analysis (e.g., metagenomics analysis) using sequencing data, particularly where sample or process variation confounds accurate quantification of predefined categories represented in samples (e.g., organismal populations from next-generation sequencing data). Advantageously, technical solutions (e.g., computing systems, methods, and non-transitory computer readable storage mediums) for addressing the above identified problems are provided in the present disclosure.

As discussed above, variations in samples or sequencing processes can impede the analysis and interpretation of corresponding sequencing data, including the profiling of microbial populations for metagenomics. For example, the accurate characterization (e.g., quantification) of microbial populations within a specimen plays a major role in understanding microbial diversity and its relationship with health and disease. Conventional methods for quantification of populations using sequencing data rely on laborious, assay-specific, and/or target-specific methods, including, for example, external titration studies using quantified standards to derive one or more quantitative standard curve models, performing quantification in a reaction separate from the sequencing assay, using an assay- or template-specific quantification standards, using a competitive template as a quantification standards, and/or relative quantification. Thus, there is a need in the art for improved systems and methods that allow for the quantification of predefined categories of populations (e.g., organisms) represented in a sample using sequencing data, and that further overcome the above limitations arising from inter-sample variation.

Accordingly, the present disclosure provides a method for determining an amount of a predefined category represented in a sample. The method includes obtaining a sample including nucleic acid molecules from the organism (e.g., a sample that is contaminated and/or infected by a microorganism). A known quantity of an internal control material is added to the sample, and the mixture of the sample with the internal control material is sequenced (e.g., by next-generation sequencing). After sequencing, sequence reads from the organism and the internal control material are counted and normalized (e.g., based on a target nucleotide sequence length). The amount of the organism in the sample is then quantified based on the first read count, the second read count, and the known quantity of the internal control material.

The systems and methods disclosed herein overcome the abovementioned deficiencies by providing a method for quantification (e.g., absolute quantification) of a predefined category (e.g., a microorganism) represented in the sample. For example, the limitations of sample and/or process variation are avoided by the addition of the internal control material to the sample prior to sequencing, such that any manipulations (e.g., sample loss, sample preparation, extraction, amplification, nucleic acid recovery, purification, library preparation, and/or sequencing) to which the sample including the organism is exposed are likewise reflected in the internal control material and the corresponding sequence reads originating from the internal control material. Furthermore, the systems and methods disclosed herein can be used for quantification of any number of samples or sample types, including any number of microbial populations, without the need for customization of the internal control material or laborious external titration assays. For example, the addition of the internal control material to each respective sample in one or more samples prior to sequencing provides that any manipulations experienced by the respective sample is likewise reflected in its corresponding internal control material, and thus each sample can be individually analyzed (e.g., for quantification of a respective one or more predefined categories included in the sample) using its respective corresponding internal control material.

For instance, improvements of the disclosed systems and methods over conventional methods are illustrated in the Examples section, below. In particular, as described below in FIG. 4 and Example 3, concentrations of the respective pathogens determined using the methods provided herein exhibited robust agreement with known concentrations of common pathogens (e.g., Staphylococcus aureus, Enterococcus faecalis, and SARS-CoV-2). In particular, the calculated concentrations were obtained without the use of the external, assay-specific, and/or template-specific quantification employed by conventional methods described above.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

One aspect of the present disclosure provides a method for determining an amount of a predefined category represented in a sample, the method including obtaining a sample containing one or more nucleic acid molecules originating from the organism and one or more nucleic acid molecules originating from a source other than the organism, and adding to the sample a known quantity of an internal control material containing one or more nucleic acid molecules.

The method further includes obtaining, in electronic form, a sequencing dataset comprising a first plurality of sequence reads and a second plurality of sequence reads from a sequencing of the sample including the internal control material, where each respective sequence read in the first plurality of sequence reads is determined by a sequencing of a nucleic acid molecule in the one or more nucleic acid molecules originating from the organism, and each respective sequence read in the second plurality of sequence reads is determined by a sequencing of a nucleic acid molecule in the one or more nucleic acid molecules in the internal control material.

A first read count for the number of sequence reads originating from the organism is determined from the first plurality of sequence reads, where the first read count is normalized based on a first target nucleotide sequence length, and a second read count for the number of sequence reads originating from the internal control material is determined from the second plurality of sequence reads, where the second read count is normalized based on a second target nucleotide sequence length. The amount of the organism in the sample is calculated, based on the first read count, the second read count, and the known quantity of the internal control material.

In some embodiments, the calculation of the organism quantity is determined by the equation Q_{org}=(Q_{IC}*RC_{org})/RC_{IC}, where Q_{org} is the amount of the organism in the sample, Q_{IC} is the known quantity of the internal control material, RC_{org} is the first normalized read count for the number of sequence reads originating from the organism, and RC_{IC} is the second normalized read count for the number of sequence reads originating from the internal control material.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is an example block diagram illustrating a computing device and related data structures used by the computing device in accordance with some implementations of the present disclosure.

FIG. 2 illustrates an example method in accordance with an embodiment of the present disclosure, in which optional steps are indicated by broken lines.

FIG. 3 illustrates an example workflow of a method in accordance with some embodiments of the present disclosure.

FIGS. 4A, 4B, and 4C illustrate performance measures obtained using the disclosed systems and methods, in accordance with some embodiments of the present disclosure. FIGS. 4A and 4B provide comparisons of calculated concentrations with known concentrations of pathogens in titration samples. FIG. 4C illustrates SARS-CoV-2 data obtained from clinical samples.

FIG. 5 illustrates viral load correlation in plasma versus quantitative PCR for two example organisms (left panel: cytomegalovirus; right panel: BK polyomavirus) in accordance with some embodiments of the present disclosure.

FIGS. 6A and 6B illustrate application of correction factors to target nucleotide sequences of an organism, such that calculated quantification is corrected to match expected quantification of the organism in accordance with some embodiments of the present disclosure.

### DETAILED DESCRIPTION

### Introduction

As sequencing costs drop, analytic operations can be automated with significant price reductions. Large-scale sequencing technologies, such as next-generation sequencing (NGS), have afforded the opportunity to achieve sequencing at costs that are less than one U.S. dollar per million bases, and, in fact, costs of less than ten U.S. cents per million bases have been realized. See, Nimwegen et al. (2016), “Is the $1000 Genome as Near as We Think? A Cost Analysis of Next-Generation Sequencing,” Clin Chem 62(11): 1458-1464, doi:10.1373/clinchem.2016.258632. Accordingly, NGS instruments are capable of generating large amounts of data (e.g., in the gigabase- to terabase-scale), for which analysis is often computationally taxing. In addition, NGS components and processes such as sample type, sample preparation, amplification, and sequencing, and the data obtained from these processes, can include a number of confounding factors that introduce variation between datasets (e.g., experiment to experiment, lab to lab, etc.) and thus hinder the analysis and comparison of such data. For instance, samples may not be uniformly prepared for sequencing due to human and/or systematic errors. In another instance, samples may not be uniformly sequenced due to the presence of nucleic acids from one or more sub-populations in the sample (e.g., microorganisms) at varying concentrations and/or having varying nucleotide lengths. Clinical samples may include large amounts of host DNA (e.g., human DNA) in addition to nucleic acids originating from one or more sub-populations (e.g., microbial, fetal, cancer, and/or other cell populations) of interest. Non-limiting examples of such clinical samples include sputum, feces, or blood culture media, which can contain nucleic acids originating from one or more of a host (e.g., human) and/or one or more sub-populations of predefined categories (e.g., infecting or contaminating microorganisms, fetal cells, cancer cells, etc.), where sub-population loads range from approximately 0-10¹³ units per milliliter of sample, or more typically approximately 10³-10⁹ units/mL.

One common practice in next-generation sequencing comprises pooling together sequencing libraries from multiple samples for simultaneous sequencing. This practice can provide an added benefit of faster sequencing times and higher throughput but is nevertheless accompanied by a dramatic increase in the amount of data collected per sequencing run, further compounding the high computational burden of NGS data analysis and interpretation. As described above, variation can be introduced at any point prior to pooling and sequencing, such that each individual sample in a pool of samples may suffer from varying inconsistencies between one or more other samples even within the same sequencing run. As a result, in some instances, data corresponding to individual samples in the pool of samples may not be suitable for direct comparison. In some such instances, additional data processing methods are needed to segregate each subset of data for individual alignment and analysis.

Such disadvantages limit the ready applicability of NGS data, at least in part because inter-sample or inter-experiment variations in the data hamper accurate quantification of sub-populations of predefined categories (e.g., genetic variations, microorganisms, fetal cells, cancer cells, etc.) represented in a sample and, similarly, whether the predefined category is present at a concentration above a given threshold (e.g., a clinically relevant threshold). As such, the ease with which NGS data can be meaningfully translated into actionable decisions (e.g., clinical decisions) is reduced.

Thus, there is a need in the art for methods of quantifying nucleic acids in a sample using sequencing data (e.g., next-generation sequencing data), particularly where one or more nucleic acids in the sample originate from different sources (e.g., populations of predefined categories, such as an organism of interest in a host specimen).

Benefit

Quantification of nucleic acids in a sample can provide valuable information relating to epidemiology (e.g., disease tracking and/or transmission), disease progression or monitoring, and/or treatment efficacy (e.g., effect of antimicrobial treatment on microbial community profiles). In such instances, comparisons are made between multiple samples from a single subject (e.g., longitudinally) or between multiple subjects, where the disadvantages of sample and dataset variation become even more apparent. Differences in sample processing and/or sequencing efficiency can also create complications when attempting to isolate and/or quantify nucleic acids derived from predefined categories of sub-populations relative to those derived from a host, or when differentiating between multiple populations of different predefined categories (e.g., co-infecting microorganisms) within a single sample, where the relative amounts of nucleic acids from two or more sources can vary widely (e.g., linear, non-linear, and/or linear within a given dynamic range). One example application of nucleic acid quantification in samples includes metagenomics, the genomic analysis of a population of microorganisms.

Metagenomics makes possible the profiling of microbial communities in the environment and the human body at unprecedented depth and breadth. Its rapidly expanding use has provided new insights into microbial diversity in natural and man-made environments and highlighted the role of microbial community profiles in health and disease applications such as infectious disease testing, pathogenesis (e.g., the interplay between acute infection and colonization), transmission risk, treatment response, disease monitoring and epidemiology, diagnosis and reporting, analysis pipeline validation, regulatory purposes, and/or other areas of clinical, diagnostic, and environmental interest.

Advantageously, in some clinical and laboratory environments, the use of metagenomics reduces sample loss and degradation and increases the sensitivity of detection by eliminating the need for in vitro microbial culture. For instance, sample loss or degradation can occur through, e.g., improper storage or handling of samples during sample collection, preparation or culture. Furthermore, a vast majority of microorganisms have not been adapted to in vitro culture, while other rare and/or novel microorganisms cannot be readily cultured. It is estimated that less than 1% of microorganisms present in the environment can be cultured in vitro. See, e.g., Streit and Schmitz (2004), “Metagenomics—the key to the uncultured microbes,” Curr Op Microb 7, 492-498, doi:10.1016/j.mib.2004.08.002. Loss of detectable microorganisms can also occur in hospital settings prior to sample collection, such as in instances where patients undergo treatment (e.g., an antibiotic therapy) immediately after admission and initial diagnosis. In such cases, patient samples collected after antibiotic exposure may not be suitable for laboratory culture, and subsequently detected microorganisms may not be representative of the actual in vivo composition of pathogens. See, Harris et al. (2017), “Influence of Antibiotics on the Detection of Bacteria by Culture-Based and Culture-Independent Diagnostic Tests in Patients Hospitalized With Community-Acquired Pneumonia,” Open Forum Infect Dis 4(1), doi:10.1093/ofid/ofx014. Through the application of metagenomics, the ability to detect rare or low-abundance pathogens can improve diagnostic applications, for instance where the cause of a disease is unknown and diagnostic panels are unable to provide information as to the etiology of the disease or provide guidelines as to appropriate treatment. See, for example, Greninger (2018), “The challenge of diagnostic metagenomics,” Expert Rev Mol Diagn 18:7, 605-615, doi:10.1080/14737159.2018.1487292.

To date, most microbial quantification studies have relied on PCR amplification of microbial marker genes (e.g., bacterial 16S rRNA), for which large, curated databases have been established, or dideoxy DNA “Sanger” sequencing. However, while conventional pathogen-specific nucleic acid amplification tests are highly sensitive and specific, they require prior knowledge of common pathogens likely to be identified in biological or environmental samples, such as those included in limited diagnostic panels. Furthermore, because Sanger sequencing is performed on single amplicons, the throughput of Sanger sequencing is limited, and large-scale Sanger sequencing projects are expensive and laborious. In contrast, NGS technologies used for metagenomics encourage a comprehensive approach to characterization of the microbiome by reducing bias, improving detection of less abundant taxa, and facilitating the discovery of novel pathogens and pathogenic markers, albeit with concomitant limitations.

For example, many of the pathogens targeted in diagnostic assays can be found in the environment and as commensals at the site of sample collection. In diseases such as pneumonia, the most frequently encountered bacterial pathogens may also exist as “normal flora” of the oropharyngeal passage, which is often itself the site of sample collection (e.g., sputum and tracheal aspirates and/or nasopharyngeal swab (NPS)) or the route for collection of more invasive specimens such as bronchoalveolar lavage (BAL). Frequent contamination by or co-collection of normal flora is essentially unavoidable in such cases. In such a scenario the diagnostic power of NGS may be limited by the fact that clinically relevant organisms cannot be readily distinguished from commensals or contamination due to the likelihood that NGS can detect the presence of both highly and minimally concentrated organisms (e.g., NGS has an almost limitless dynamic range) without providing a great deal of inherent context to interpret the clinical relevance of detections in the sequencing data. Thus, NGS may detect the presence of a pathogen (e.g., nucleic acids from a pathogen) and its relative abundance (e.g., percent abundance) to other detected nucleic acids or organisms without providing any indication of whether or not the detected pathogen is present at a clinically relevant concentration.

The traditional practice in microbiological laboratories has been to perform semi-quantitative or quantitative cultures to distinguish pathogenic loads of organisms (e.g., bacteria) from non-clinically relevant commensal carriage. Different diagnostic titer guidelines exist for different types of specimens. Similar approaches have been applied to NGS assays. Typically, NGS provides semi-quantitative data, where, in the absence of confounding factors such as sample preparation errors or differences in sequencing efficiency, the number of sequence reads for a target is generally related to the abundance of the target. Conventional methodology has made use of this relationship to obtain relative quantification data for nucleic acids of interest in NGS. For example, the relative abundance of nucleic acids in a sample can be determined by performing a series of serial dilutions (e.g., 10-fold dilutions) of one or more samples, sequencing the series of diluted samples, and then plotting the numbers of sequence reads found in each. These methods are based on an assumption that if the relationship between the number of sequence reads in the serial diluted samples has a linear relationship (e.g., a 10-fold dilution results in an approximately 10-fold reduction in the number of sequence reads, a 100-fold dilution results in an approximately 100-fold reduction in the number of sequence reads, etc.), then the number of sequence reads can be used to relatively quantify different targets present in the sample (e.g., to relatively quantify high and low concentration targets). For instance, if a first sequenced nucleic acid has 10 sequencing reads and a second has 100 sequencing reads, it is concluded that the second nucleic acid is 10 times more concentrated than the first. This method can be used, for example, to detect gene duplication and/or to determine the number of copies of a gene in a genome. Nonetheless, this approach is merely relative and, as a result, fails to determine the actual concentration of either the first or the second nucleic acid. Furthermore, resolution can decrease at very low and/or very high concentrations, such that relative concentrations estimated over a large range (e.g., over several orders of magnitude) may not faithfully reflect actual abundance. Generally, this approach is subject to the disadvantages of relative quantification described above, due to its lack of accurate quantification and failure to account for intra-lab and inter-lab variations.

In contrast, absolute quantification of NGS data provides information on the number of genomic and/or transcriptomic copies of nucleic acids (e.g., for one or more RNA and/or DNA targets) in a volume or weight of specimen, including but not limited to copies (e.g., genomic and/or transcriptomic copies) per mL, genomic equivalents (GE)/mL, and/or copies per weight of specimen (e.g., mg). Absolute quantification within the context of NGS data analysis traditionally requires upfront (e.g., external) titration studies with quantified standards to derive one or more quantitative standard curve models. Specimens with unknown quantities of genomic and/or transcriptomic targets (e.g., nucleic acids derived from organisms of interest) can then be assessed using the derived model(s).

For example, a common approach to absolute quantification includes quantifying the nucleic acids in a sample used for NGS in a separate reaction. In some such instances, quantitative PCR (qPCR) is used for absolute quantification, using a standard curve approach. In this approach, a standard curve generated from plotting the crossing point (Cp) values obtained from real-time PCR against known quantities of a single reference template provides a regression line that can be used to extrapolate the quantities of the same target gene in samples of interest. Serial dilutions (e.g., 10-fold dilutions) of the reference template are set up alongside samples containing the specific gene target to be quantified. Various separate reactions are run, including one for each level of the reference target and one for each of the samples of interest. Additionally, in some instances, separate standard curves with separate reference templates are obtained for different gene targets, to account for the effect of assay-specific differences in PCR efficiencies on quantification.

A limitation of this approach and other external titration studies is that the one or more derived models are specific to the particular assay or target (e.g., sample and/or organism of interest), and thus require customization for each respective specimen processing protocol, nucleic acid extraction efficiency, target pathogen, molecular target, and/or any other component, parameter, or process utilized during data acquisition. Therefore, any changes in specimen processing protocols or other such variables will likely require one or more new titration studies and derivation of a corresponding one or more new standard curve models. This process is laborious, time-consuming, and costly, particularly where, in the context of metagenomics and other applications of high-throughput sequencing analysis, it is desirable to perform detection and/or characterization of a large number of sub-populations (e.g., microorganisms) within a large number of samples. Furthermore, difficulties can arise in instances where one or more populations of interest include novel targets and a reference sequence for generating a target-specific quantification standard model is unavailable.

As a further illustrative example, the power of NGS lies in its massive parallelism (e.g., at least 10, at least 100, and/or at least 1000 samples can be processed simultaneously and in parallel). Using qPCR to quantify a plurality of candidate targets (e.g., a theoretically unlimited number of known and/or novel microorganisms to be detected and quantified) in each of the many possible samples requires a substantial and prohibitive amount of human labor. Although quantification of targets using hundreds and sometimes thousands of separate nucleic acid reactions has been performed using qPCR (see, e.g., Hindson et al., 2011, “High-Throughput Droplet Digital PCR System for Absolute Quantitation of DNA Copy Number,” Anal Chem. 83(22): 8604-8610), this approach is technically challenging and requires special equipment. Additionally, qPCR approaches generally assume or require the assays to have the same PCR efficiency in singleplex and multiplex reactions, which further limits the universality of this approach. Notably, all standard curve-based quantification approaches published to date require setting up external reactions and the calculation of standard curves.

Another approach to quantifying nucleic acids from NGS data uses assay-specific competitive templates (see, e.g., U.S. Patent Publication 2015/0292001, “Methods for Standardized Sequencing of Nucleic Acids and Uses Thereof,” published Oct. 15, 2015). Such methods aim to provide reproducibility in measurements of nucleic acid copy number in samples by relying on a proportional relationship of a native target sequence to a respective competitive internal amplification control specifically designed for that native target sequence. However, such approaches are assay- and template-specific (e.g., the competitive template is target- and sample-specific) and require the design of new competitive internal amplification controls for each assay and/or template to be sequenced, limiting the general applicability of this approach. In addition, the competitive template approach requires that the target be sequenced with and without the competitive template in order to deconvolute the sequencing response of the target alone from the sequencing response of the target plus the competitive template. This effectively doubles the number of sequencing reactions performed, thus increasing the cost and labor involved, adds to the level of complexity of the approach and has the potential to introduce additional error into the calculation.

Given the above deficiencies in conventional methods for nucleic acid quantification, there is a need for improved systems and methods for quantification of predefined categories (e.g., microorganisms, fetal cells, cancer cells, and/or other sub-populations) represented in a sample, that will overcome the above limitations.

Accordingly, the present disclosure provides systems and methods for determining an amount of a predefined category (e.g., a contaminating and/or infecting microorganism, a sub-population of fetal cells, a sub-population of cancer cells, etc.) in a sample (e.g., a clinical specimen obtained from a subject), for instance where the sample includes one or more nucleic acid molecules originating from the predefined category and one or more nucleic acid molecules originating from a source other than the predefined category (e.g., the subject). A known quantity of an internal control (IC) material is added to the sample, where the internal control material includes one or more nucleic acid molecules. The sample, together with the added IC material, is then subjected to a sequencing reaction (e.g., NGS), thus obtaining a sequencing dataset including a first plurality of sequence reads (e.g., corresponding to the one or more nucleic acids from the predefined category) and a second plurality of sequence reads (e.g., corresponding to the one or more nucleic acids from the IC material).

In an example embodiment of the method, in accordance with the present disclosure, the IC material is a reference nucleic acid (e.g., RNA or DNA) sequence comprising natural and/or synthetic nucleic acid sequences. In one embodiment, the known quantity of the IC material that is added to the sample prior to sequencing is determined based on one or more parameters of an assay. For instance, in some embodiments, the known quantity of the IC material is selected based on factors including, but not limited to, the desired resolution of the assay, the nucleic acid extraction efficiency, the concentration range of the nucleic acids to be sequenced, the prevalence of genetic mutations to be detected, and/or the desired sequencing read depth.

In another example embodiment of the method, the sample comprises tissue and/or cells. In some embodiments, the sequencing of the sample and the IC material further includes extracting nucleic acids (e.g., RNA or DNA) from the combined sample and IC material. In some embodiments, the extracted nucleic acids are prepared for sequencing (e.g., fragmented, reverse-transcribed, and/or converted into a sequencing library by annealing and/or ligation to sequencing adaptors and molecular barcodes). In some embodiments, sequencing is performed by next-generation sequencing, including any suitable method known in the art (e.g., Illumina, Life Technologies, Roche, Pacific Biosciences, etc.).

The method further includes determining a first read count from the first plurality of sequence reads and a second read count from the second plurality of sequence reads, where the first and second read counts are normalized based on a first target nucleotide sequence length (e.g., corresponding to the predefined category) and a second target nucleotide sequence length (e.g., corresponding to the IC material), respectively. The amount of the predefined category in the sample is then calculated based on the first read count, the second read count, and the known quantity of the internal control material. For example, in some embodiments, the calculation of the predefined category quantity is determined by the equation Q_{org}=(Q_{IC}*RC_{org})/RC_{IC}, where Q_{org} is the amount of the predefined category in the sample, Q_{IC} is the known quantity of the internal control material, RC_{org} is the first normalized read count for the number of sequence reads originating from the predefined category, and RC_{IC} is the second normalized read count for the number of sequence reads originating from the internal control material.

The systems and methods disclosed herein overcome the limitations of sample and/or process variation via the addition of a known quantity of IC material to the sample prior to sample processing and sequencing, which is then carried through all sample processing and sequencing procedures. In particular, any manipulations (e.g., sample loss, sample preparation, extraction, amplification, nucleic acid recovery, purification, library preparation, and/or sequencing) to which the sample (e.g., including the predefined category) is exposed are likewise experienced by the IC material, and the number of sequence reads obtained from sequencing nucleic acid molecules from the IC material (e.g., the second read count) will also reflect all of the manipulations and systematic losses reflected in the sequence reads obtained from the predefined category (e.g., the first read count).

Furthermore, the systems and methods disclosed herein can be used for quantification of any number of samples or sample types, including any number of predefined categories (e.g., microbial populations). For example, in some embodiments, the provided systems and methods are used to quantify a plurality of populations of predefined categories (e.g., organisms and/or microorganisms) within a single sample. While quantification of microorganisms for metagenomics has been described above as an illustrative example, the presently disclosed systems and methods are not limited to quantification of microorganisms but are applicable to any predefined category or sub-population that can be represented by nucleic acid molecules in a sample, such as a population of cells, a population of organisms, a tissue, and/or a cell type or origin (e.g., a population of microorganisms, cancer cells, fetal cells, etc.). Thus, the systems and methods disclosed herein can be used for quantification of any predefined category represented in a sample, including but not limited to microorganisms.

In some embodiments, the provided systems and methods are used to quantify one or more populations of predefined categories within each sample in a plurality of samples. In some embodiments, a corresponding known quantity of IC material is added to each respective sample in a plurality of samples, and the plurality of samples are pooled prior to sample processing and sequencing. In some such instances, quantification of one or more predefined categories within each sample in the pooled plurality of samples can be performed without the need for additional customization of the IC material or other external titration studies. For example, the addition of the IC material to each respective sample in the one or more samples prior to sequencing provides that any manipulations experienced by the respective sample is likewise reflected in its corresponding IC material, and thus, for each respective sample, quantification of a respective one or more predefined categories can be separately performed using its respective corresponding IC material.

The systems and methods provided herein overcome the limitations of conventional methods for quantification of sequencing data. By calculating the amount of the predefined category in the sample using normalized read counts for the predefined category, normalized read counts for the IC material, and the known initial quantity of the IC material, accurate quantification (e.g., absolute quantification) of a predefined category (e.g., a microorganism) in the sample is achieved. Such quantitative data can be used for data comparison, analysis, and/or decision-making, including those relating to infectious disease testing, pathogenesis, transmission risk, treatment response, disease monitoring and epidemiology, diagnosis, reporting, analysis pipeline validation, regulatory purposes, and/or other areas of clinical, diagnostic, and environmental interest. Furthermore, by providing absolute quantification using known quantities of IC material, the systems and methods provided herein are not subject to the limitations of relative quantification methods, which suffer from inaccurate estimations of fold differences and a lack of actionable quantitative data. In some embodiments, the disclosed methods are performed without the need for external titration studies, thus saving labor, time and cost for each sequencing run and subsequent analysis, and further improve upon conventional assay-specific, template-specific, and/or target-specific methods for quantification due to their applicability across a wide variety of samples and targets without the need for extensive or repetitive methods for generating models or constructing standard curves. Similarly, the provided methods improve upon conventional quantification methods that rely on reference templates to construct standard curves, thus allowing the method to be used for the detection and quantification of novel categories and/or populations, such as microorganisms, fetal cells, and/or cancer cells.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the term “microorganism,” or “microbe,” refers to a microscopic organism. In some embodiments, the term “microorganism” will be understood to include bacteria, fungi, protozoa (e.g., protozoan parasites), viruses (e.g., DNA viruses and/or RNA viruses), algae, archaea, phages, and/or helminths (e.g., multicellular eukaryotic parasites). In some embodiments, a microorganism is a single-celled organism and/or a colony of single-celled organisms. In some embodiments, a microorganism is eukaryotic or prokaryotic. In some embodiments, a microorganism is a pathogen (e.g., disease-causing), such as a human, animal, or plant-infective pathogen.

Examples of bacteria include, but are not limited to, disease-causing agents such as Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp (such as Bacillus anthracis, Bacillus cereus, Bacillus subtilis, Bacillus thuringiensis, and Bacillus stearothermophilus), Bacteroides sp. (such as Bacteroides fragilis). Bartonella sp. (such as Bartonella bacilliformis and Bartonella henselae), Bifidobacterium sp. Bordetella sp (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp (such as Burkholderia pseudomallei and Burkholderia cepacia), Campylobacter sp. (such as Campylobacter jejuni, Campylobacter coli, Campylobacter lari and Campylobacter fetus), Capnocytophaga sp. Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeium and Corynebacterium), Clostridium sp. (such as Clostridium perfringens, Clostridium difficile, (Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enter aggregative E. coli and uropathogenic E. coli), Enterococcus sp. (such as Enterococcus faecalis and Enterococcus faecium), Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus), Helicobacter sp. (such as Helicobacter pylori, Helicobacter cinaedi and Helicobacter fennelliae), Kingella kingii, Klebsiella sp (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia haemolytica, Microsporum canis, Moraxella catarrhalis, Morganella sp., Mobiluncus sp., Micrococcus sp., mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasma sp (such as Mycoplasma pneumonia, Mycoplasma hominis, and Mycoplasma genitalum), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly; Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella choleraesuis and Salmonella typhimurium), Serratia sp (such as Serratia marcescans and Serratia liquefaciens), Shigella sp. (such as Shigella dysenteriae. Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equisimilis, Group D streptococci, Streptococcus bovis, Group F streptococci, and Streptococcus anginosus Group G streptococci), Spirillum minus, Streptobacillus moniliforme, Treponema sp. (such as Treponema carateum, Treponema petnue, Treponema pallidum and Treponema endemicum), Trichophyton rubrum, T. mentagrophytes, Tropheryma whippelii, Ureaplasma urealyticum, Veillonella sp., Vibrio sp. (such as Vibrio cholerae, Vibrio parahaemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia.

Examples of fungi include, but are not limited to, Aspergillus sp., Candida auris, Candida albicans, Candida dubliniensis, Candida famata, Candida glabrata, Candida guilliermondii, Candida kefyr, Candida lusitaniae, Candida krusei, Candida parapsilosis, Candida tropicalis, Cryptococcus gattii, Cryptococcus neoformans, Fusarium sp., Malassezia furfur, Rhodotorula sp., Trichosporon sp., Histoplasma capsulatum, Coccidioides immitis, and Pneumocystis carinii, as well as the causative agents of Aspergillosis, Balsomycosis, Candidiasis, Coccidioidomycosis, fungal eye infections, fungal nail infections, histoplasmosis, mucormycosis, mycetoma, Pneuomcystis pneumonia, ringworm, sporotrichosis, crypococcosis, and Talaromycosis.

Examples of protozoan parasites include, but are not limited to, Plasmodium falciparum, P. vivax, P. ovals P. malariae, P. berghei, Leishmania donovani, L. infantum, L. chagasi, L. mexicana, L. amazonensis, L. venezuelensis, L. tropica, L. major, L. minor, L. aethiopica, L. Biana braziliensis, L. (V.) guyanensis, L. (V) panarmensis, L. (V.) periviana, Trypanosoma brucei rhodesiense, T. brucei gambiense, T. cruzi, Giardia intestinalis. G. lamblia, Toxoplasma gondii, Entamoeba histolytica, Trichomonas vaginalis, Pneumocystis carinii, and Cryptosporidium parvum.

Examples of helminths include, but are not limited to, Filarioidea sp., Wuchereria sp. (such as Wuchereria bancrofti), Brugia sp. (such as Brugia malayi and Brugia timori), Loa sp. (such as Loa loa), Mansonella sp. (such as Mansonella streptocerca, Mansonella perstans, and Mansonella ozzardi), Onchocerca sp. (such as Onchocerca volvulus), Enterobius vermicularis, Ascaris sp. (such as Ascaris lumbricoides), Dracunculus (such as Dracunculus medinensis), Ancylostoma sp. (such as Ancylostoma duodenale, Ancylostoma braziliense, Ancylostoma tubaeforme, and Ancylostoma caninum), Necator sp. (such as Necator americanus), Trichuris sp. (such as Trichuris trichiura, Trichuris vulpis, Trichuris campanula, Trichuris suis, and Trichuris muris), Strongyloides sp. (such as Strongyloides stercoralis, Strongyloides canis, Strongyloides fuelleborni, Strongyloides cebus, and Strongyloides kellyi), Nematodirus sp., Moniezia sp., Oesophagostomum sp. (such as Oesophagostomum bifurcum, Oesophagostomum aculeatum, Oesophagostomum brumpti, Oesophagostomum stephanostomum, and Oesophagostomum stephanostomum var thomasi), Cooperia sp. (such as Cooperia ostertagi and Cooperia oncophora), Haemonchus sp., Ostertagia sp. (such as Ostertagia ostertagi), Trichostrongylus sp. (such as Trichostrongylus axei), Dirofilaria sp. (such as Dirofilaria immitis, Dirofilaria tenuis and Dirofilaria repens), and Schistosoma sp. (such as Schistosoma incognitum, Schistosoma ovuncatum, Schistosoma sinensium. Schistosoma indicum, Schistosoma nasale, Schistosoma spindale, Schistosoma japonicam, Schistosoma malayensis, Schistosoma mekongi, Schistosoma haematobium. Schistosoma bovis, Schistosoma curassoni, Schistosoma guineensis, Schistosoma haematobium, Schistosoma intercalatum, Schistosoma leiperi, Schistosoma margrebowiei, Schistosoma mattheei, Schistosoma mansoni, Schistosoma edwardiense, Schistosoma hippotami, and Schistosoma rodhaini)

Examples of viruses include, but are not limited to, disease-causing agents such as Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Coronavirus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, Norovirus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Severe acute respiratory syndrome coronavirus 2, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, the term “microorganism” will be understood to include any one or more bacteria, fungi, protozoa, viruses, algae, archaea, phages, and/or helminths selected from a database (e.g., a microbial genome database, a transcriptomic database, a proteomic database, a metabolomics database, a taxonomic database, and/or a clinical database). In some embodiments, the database comprises one or more entries corresponding to and/or identifying a microorganism (e.g., an annotation, for a respective microorganism, to a genome, transcriptome, nucleic acid sequence, protein sequence, metabolite, taxonomic record and/or clinical record). In some embodiments, a microorganism is selected from a database that is locally maintained, proprietary, and/or open-access. In some embodiments, a microorganism is selected from a national and/or international database. Examples of such databases include, but are not limited to, NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. For example, MBGD comprises all complete genome sequences of bacteria, archaea, and unicellular eukaryotes, including fungi and protozoa, available at the NCBI genomes site. The Microbial Rosetta Stone is a database that provides information on disease-causing organisms (e.g., bacteria, fungi, protozoa, DNA viruses, RNA viruses, plants, and animals) and the toxins produced therefrom. See, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gky1054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19; each of which is hereby incorporated by reference herein in its entirety.

As used herein, the terms “antimicrobial resistance marker” or “AMR marker” refers to a measurable and/or detectable marker indicating that a respective microorganism has antimicrobial resistance. As used herein, the term “antimicrobial resistance” refers to a property of or exhibited by a respective microorganism, such that the respective microorganism is resistant to one or more antimicrobial interventions (e.g., where an effect of an antimicrobial intervention is attenuated, obstructed, or negated). As used herein, the term “antimicrobial susceptibility” refers to a property of or exhibited by a respective microorganism, such that the respective microorganism is susceptible to one or more antimicrobial interventions (e.g., where an effect of an antimicrobial intervention serves to kill, diminish, slow or prevent growth in one or a population of microorganisms).

In some embodiments, antimicrobial resistance is conferred by a genetic sequence (e.g., an antimicrobial resistance gene). In some embodiments, the antimicrobial resistance marker is a genetic marker (e.g., a nucleic acid sequence for the antimicrobial resistance gene indicating that the gene comprises a mutation that confers resistance). In some embodiments, the antimicrobial resistance marker is a restriction fragment length polymorphism (RFLP), a random amplified polymorphic DNA (RAPD), an amplified fragment length polymorphism (AFLP), a variable number tandem repeat (VNTR), an oligonucleotide polymorphism (OP), a single nucleotide polymorphism (SNP), an allele specific associated primer (ASAP), an inverse sequence-tagged repeat (ISTR), an inter-retrotransposon amplified polymorphism (IRAP), and/or a simple sequence repeat (SSR or microsatellite). In some embodiments, an antimicrobial resistance marker is detected based on a mapping (e.g., an alignment) of one or more sequence reads to a reference sequence (e.g., a reference genome). In some embodiments, an antimicrobial resistance marker is an amino acid sequence and/or an amino acid residue. In some embodiments, an antimicrobial resistance marker is a biochemical marker.

In some embodiments, an antimicrobial resistance marker indicates that a respective microorganism is resistant to one or more interventions for a corresponding type of microorganism (e.g., antibacterial resistance, antiprotozoal resistance, antifungal resistance, anihelminthic resistance, and/or antiviral resistance). For example, in some embodiments, an antimicrobial intervention is a drug that targets a specific gene in a respective microorganism, and a mutation in the gene confers resistance to the microorganism. In some such embodiments, an antimicrobial resistance marker can be a genetic marker for the target gene that indicates a resistance to the antimicrobial drug.

As used herein, the term “antimicrobial resistance status” refers to an indication of a presence or absence of an antimicrobial resistance marker. For example, the term antimicrobial resistance status or AMR status will be understood to include an indication that a respective biological sample and/or a microorganism detected in a biological sample has either antimicrobial resistance or antimicrobial susceptibility. In some embodiments, an antimicrobial resistance status includes an indication that an antimicrobial resistance marker is present (e.g., has been detected) in the respective biological sample and/or microorganism. In some embodiments, an antimicrobial resistance status includes an indication of any one or more features for the respective antimicrobial resistance marker (e.g., gene identifier, gene name, intervention (drug) information, intervention (drug) classes, associated organisms, gene families, and/or resistance mechanisms).

In some embodiments, an antimicrobial resistance marker is associated with one or more microorganisms in a plurality of microorganisms (e.g., where the respective microorganism has been reported or annotated as expressing the respective antimicrobial resistance marker). In some embodiments, a first antimicrobial resistance marker is associated with a first respective microorganism in a plurality of microorganisms, and a second antimicrobial resistance marker is associated with a second respective microorganism, other than the first microorganism, in the plurality of microorganisms.

Examples of antimicrobial resistance markers (e.g., genes and/or amino acid residues) include, but are not limited to, the antimicrobial resistance markers listed below in Table 1.

TABLE 1
Example Antimicrobial Resistance Markers

| Intervention | |
|---|---|
| Type | Marker: Gene Name or Subtype [AA Mutation] |
| Antibiotic | Aminocoumarins: GyrB, ParE, ParY |
| Resistance | Aminoglycosides: AAC(1), AAC(2′), AAC(3), AAC(6′), ANT(2″), |
| | ANT(3″), ANT(4″), ANT(6), ANT(9), APH(2″), APH(3″), APH(3′), |
| | APH(4), APH(6), APH(7″), APH(9), ArmA, RmtA, RmtB, RmtC, Sgm |
| | β-Lactams: AER, BLA1, CTX-M, KPC, SHV, TEM; BlaB, CcrA, |
| | IMP, NDM, VIM; ACT, AmpC, CMY, LAT, PDC; OXA β-lactamase; |
| | methicillin-resistant PBP2; antibiotic-resistant Omp36, OmpF, PIB |
| | (por); bla (blaI, blaR1) and mec (mecI, mecR1) operons |
| | Chloramphenicol: CAT; Chloramphenicol phosphotransferase |
| | Ethambutol: EmbB |
| | Mupirocin: MupA, MupB |
| | Peptide antibiotics: MprF |
| | Phenicol: Cfr 23S rRNA methyltransferase |
| | Rifampin: Arr; Rifampin glycosyltransferase; Rifampin |
| | monooxygenase; Rifampin phosphotransferase; DnaA, RbpA; RpoB |
| | Streptogramins: Cfr 23S rRNA methyltransferase; ErmA, ErmB, |
| | Erm(31); Lsa, MsrA, Vga, VgaB; Streptogramin Vgb lyase; Vat |
| | acetyltransferase |
| | Fluoroquinolones: Fluoroquinolone acetyltransferase; |
| | Fluoroquinolone-resistant GyrA, GyrB, ParC; Qnr |
| | Fosfomycin: FomA, FomB, FosC; FosA, FosB, FosX |
| | Glycopeptides: VanA, VanB, VanD, VanR, VanS |
| | Lincosamides: Cfr 23S rRNA methyltransferase; ErmA, ErmB, |
| | Erm(31); Lin |
| | Linezolid: Cfr 23S rRNA methyltransferase |
| | Macrolides: Cfr 23S rRNA methyltransferase; ErmA, ErmB, |
| | Erm(31); EreA, EreB; GimA, Mgt, Ole; MPH(2′)-I, MPH(2′)-II; |
| | MefA, MefE, Mel |
| | Streptothricin: sat |
| | Sulfonamides: Sul1, Sul2, Sul3, sulfonamide-resistant FolP |
| | Tetracyclines: Mutant porin PIB (por) with reduced permeability; |
| | TetX; TetA, TetB, TetC, Tet30, Tet31; TetM, TetO, TetQ, Tet32, Tet36 |
| | Antibiotic efflux: MacAB-TolC, MsbA, MsrA, VgaB; EmrD, |
| | EmrAB-TolC, NorB, GepA; MepA; AdeABC, AcrD, MexAB-OprM, mtrCDE, |
| | EmrE; adeR, acrR, baeSR, mexR, phoPQ, mtrR |
| Antifungal | CYP51a [F219S, F46Y, M172V, N248T, D255E, G138C, G138S, |
| Resistance | G434C, G54E, I266N, G54R, G54V, G54W, H147Y, L98H, M217I, |
| | M220L, M220T, M220V, P216L, R228Q, Y121F, T289A, G448S, |
| | M172I, Y431C] |
| | ERG11 [A114S, G487T, T916C, A61V, D116E, D225H, D225Y, |
| | E165K, E266D, F126L, F126T, F145L, F380S, F449L, F449Y, |
| | F72L, G129A, G307S, G448V, G450E, G464S, G484S, H283R, |
| | I253V, I471T, K119L, K119N, K128T, R467I, K143E, K143Q, |
| | K143R, K161N, L491V, M140R, P375Q, P49R, T486P, P503L, |
| | Q474K. R163T. R381I, R467K, S405F, T132H, T229A, T494A, |
| | V437I, V452A. V488I. V130I, Y132F, Y132H, Y136F, Y205E, |
| | G472R, Y257H, Y33C. Y39C. Y79C, T199I] |
| | tub2 [E198A, H6Y] |
| | FKS1 [D632E, D632G, D632Y, D646Y, F639I, F641S, F655C, |
| | L642S, N470K, P660A, S639F, S639P, S645F, S645P, S645Y, V641K] |
| | CYP51b [G460S, S508T] |
| | CYP51c [Y319H, T788G] |
| | MgCYP51 [L50S, V136A, Y461S, S524T, Y459C, Y459S, G460D] |
| | MfCYP51 [A313G, Y463H, Y136F, Y463D, Y461D, Y463N] |
| | FUR1 [R101C, F211I] |
| | FKS2 [F659del, F659S, F659V] |
| | BcSdhB [P225F, H272Y, H272R] |
| | CYP51 [A29P, D78Y, E106K, E331A, F506I, G459S, G511S, |
| | I381V, I440V, K23E, K449R, K508R, M144T, N244S, Q167H, |
| | Q309H, Q43H, R462H, S35T, S505Q, S507P, V37A, V55A, Y133F, |
| | Y134F, Y136F, Y136H, Y137H, Y486H] |
| | DHPS [T55A, P57S] |
| | Cytb [G143A] |
| | RTA2 [G234S] |
| | HapE [P88L] |
| | cox10 [R243Q] |
| | DHFR [D153V, S37T, I158V, V79I, Y197L, T14A, P26Q, M52I, |
| | E63G, T144A, K171E, S106P, E127G, R170G] |
| Antiprotozoal | Pfmdr1 [N86Y, Y184F, S1034C, N1042D, 1246Y] |
| Resistance | Pfcrt [K76T, C72S, M74I, N75E, A220S, Q271E, N326S, I356T, R371I] |
| | Pfmrp [Y191H, A437S] |
| | Pfnhe1 [ms4670] |
| | PfATP4 [G223R] |
| | Pfdhps [S436A/F, A437G, K540E, A581G, A613T/S, A16V, N51I, |
| | C59R, I164L] |
| | PfAtp18 [T38I] |
| | PfK13 [Y493H, R539T, I543T, C580Y, M476I, D56V, F446I, P574L] |
| | Pfcytb [Y268S/C/N] |
| | MRP1, HSP70, PRP1 (Leishmania) |
| | LdMT [L856P, T420N, L832F, V176D, W210, Y354F, F1078Y] |
| | LdRos3 [M1] |
| Antihelminthic | beta-tubulin [F200Y, E198A. F167Y] |
| Resistance | unc-38 |
| | unc-63 |
| | acr-8 |
| | mptl-1 |
| | des-2 |
| | deg-3 |
| | avr-14 [L256F] |
| | lgc-37 [K169R] |
| | glc-5 [A169 V] |
| | ggr-3 |
| | pgpA |
| Antiviral | A H1N1 [H275Y, Q136K, N70S, I222V/M, Y155H] |
| Resistance | A H1N1 pdm09 [N294S, H275Y, I222V, I222R, E119G, E119V, |
| | N325K, S247N, I117V] |
| | A H3N2 [R292K, N294S, D151A/E, Q136K, E119V/A/D/G, |
| | R224K, R371K, R224K, E276D, H274Y, I222V] |
| | B [E119A/D/G/A, H274Y, R371K, I222T, R292K, N294S, D198N, D198E] |

See, for example, Capela et al., 2019, “An Overview of Drug Resistance in Protozoal Diseases,” Int J Mol Sci. 20(22): 5748; doi: 10.3390/ijms20225748; Beech et al., 2011, “Anthelmintic resistance: markers for resistance, or susceptibility?” Parasitology 138(2): 160-174; doi: 10.1017/S0031182010001198; and Toledu-Rueda et al., 2018, “Antiviral resistance markers in influenza virus sequences in Mexico, 2000-2017,” Infect Drug Resist 11: 1751-1756; doi: 10.2147/IDR.S153154; each of which is hereby incorporated herein by reference in its entirety.

In some embodiments, the term “antimicrobial resistance marker” will be understood to include any one or more genes, amino acid sequences amino acid residues, genetic markers, and/or biochemical markers selected from a database. In some embodiments, an antimicrobial resistance marker is selected from a database that is one or more of locally maintained, proprietary, and/or open-access. In some embodiments, an antimicrobial resistance marker is selected from a national and/or international database. Examples of such databases include, but are not limited to, the National Database of Antibiotic Resistant Organisms (NDARO), the Comprehensive Antibiotic Resistance Database (CARD), ResFinder, PointFinder, ARG-ANNOT, ARGs-OSP, PlasmoDB, the Mycology Antifungal Resistance Database (MARDy), DBDiaSNP, the HIV Drug Resistance Database, the Virus Pathogen Resource (ViPR), and/or any of the databases used for selecting one or more microorganisms, as disclosed above. See, for example, McArthur et al., 2013, “The Comprehensive Antibiotic Resistance Database,” Antimicrob Ag Chemother, 57(7) 3348-3357; doi: 10.1128/AAC.00419-13; Zankari et al., 2017, “PointFinder: a novel web tool for WGS-based detection of antimicrobial resistance associated with chromosomal point mutations in bacterial pathogens,” J Antimicrob Chemother, 72 (10) 2764-2768; doi: 10.1093/jac/dkx217; Gupta et al., 2013, “ARG-ANNOT, a New Bioinformatic Tool To Discover Antibiotic Resistance Genes in Bacterial Genomes,” Antimicrob Ag Chemother, 58 (1) 212-220; doi: 10.1128/AAC.01310-13; Zhang et al., “ARGs-OSP: online searching platform for antibiotic resistance genes distribution in metagenomic database and bacterial whole genome database,” bioRxiv 337675; doi: 10.1101/337675; Nash et al., 2018, “MARDy: Mycology Antifungal Resistance Database,” 34 (18) 3233-3234; doi: 10.1093/bioinformatics/bty321; and Mehla and Ramana, 2015, “DBDiaSNP: An Open-Source Knowledgebase of Genetic Polymorphisms and Resistance Genes Related to Diarrheal Pathogens,” OMICS 19 (6) 354-360; doi: 10.1089/omi.2015.0030; each of which is hereby incorporated herein by reference in its entirety.

As used herein, the term “sample,” “biological sample,” or “patient sample” refers to any sample taken from a subject, which can reflect a biological state associated with the subject. Examples of samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the sample consists of blood, whole blood, plasma, serum, urine, cerebrospinal fluid, fecal, saliva, sweat, tears, pleural fluid, pericardial fluid, or peritoneal fluid of the subject. A sample can include any tissue or material derived from a living or dead subject. A sample can be a liquid sample or a solid sample (e.g., a cell or tissue sample). A sample can be a cell-free sample. A sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A sample can be a stool sample. A sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A sample can be obtained from a subject invasively (e.g., surgical means) or non-invasively (e.g., a blood draw, a swab, or collection of a discharged sample). A sample can be a tissue or organ from an animal, a cell (e.g., within a subject, taken directly from a subject, and/or a cell maintained in culture or from a cultured cell line), a cell lysate, a lysate fraction, and/or a cell extract. A sample can be a solution comprising one or more molecules derived from a cell, cellular material, and/or viral material (e.g., nucleic acid). A sample can be a solution comprising a non-naturally occurring nucleic acid (e.g., a cDNA or next-generation sequencing library), which is assayed as described herein.

The term “sample” can refer to a control sample, including positive control samples, negative control samples, or blank control samples. As used herein, a positive control sample refers to a sample that comprises a known, non-zero amount of nucleic acid molecules corresponding to at least one target predefined category (e.g., microorganism of interest). In some embodiments, a positive control sample is obtained from a subject with a known population of a predefined category such as a microorganism (e.g., a pathogenic infection), or from diseased tissue in a subject diagnosed with an infectious disease. In some embodiments, the positive control sample comprises natural and/or synthetic nucleic acids. As used herein, a negative control sample refers to a sample that does not include nucleic acids corresponding to at least one respective predefined category (e.g., microorganism of interest). In some embodiments, the negative control sample is obtained from a healthy subject, or from a healthy tissue in a subject diagnosed with an infectious disease. In some embodiments, a positive or negative control sample is validated (e.g., for presence, absence, and/or quantification of a microorganism of interest and/or of a nucleic acid molecule of interest) by a laboratory validation technique, such as targeted enrichment sequencing, PCR, in vitro culture, immunoassays (e.g., ELISA, Western blot, chemiluminescence, etc.), serological assays and/or antimicrobial susceptibility assays. As used herein, a blank control sample refers to a sample that comprises one or more reagents used for processing the positive control sample and/or the negative control sample (e.g., reagents for sample collection, sample storage, pre-processing, nucleic acid isolation, and/or sequencing). In some embodiments, the blank control sample does not comprise biological material. In some embodiments, the blank control sample is water.

A first sample and a second sample can be matched samples. For example, in some embodiments, a first sample and a second sample are obtained from a diseased tissue and a healthy tissue from the same subject, respectively. In some embodiments, a first sample and a second sample are obtained from a subject diagnosed with an infectious disease and a healthy subject from the same cohort, respectively (e.g., in a clinical study). In some embodiments, a first sample and a second sample are process matched. For example, in some embodiments, a first sample and a second sample are prepared using the same process, including the reagents, equipment, processing times, and/or operator or technician used to perform the method, as well as matching workflows for sequencing, mapping, and/or pre-processing.

As used herein, the terms “nucleic acid” and “nucleic acid molecule” are used interchangeably. The terms refer to nucleic acids of any composition form, such as ribonucleic acid (RNA), deoxyribonucleic acid (DNA, e.g., complementary DNA (cDNA), genomic DNA (gDNA) and the like), and/or DNA or RNA analogs (e.g., containing base analogs, sugar analogs and/or a non-native backbone and the like). In some embodiments, nucleic acids are in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid, in some embodiments, can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As used herein, the terms “sequencing,” “sequencing reaction,” and the like refer to any biochemical processes that may be used to determine the order of biological macromolecules such as nucleic acids. For example, sequencing data can include all or a portion of the nucleotide bases in a nucleic acid molecule such as an mRNA transcript, a DNA fragment and/or a genomic locus.

As used herein, the term “sequence reads,” “sequencing reads,” or “reads” refers to nucleotide base sequences produced by any nucleic acid sequencing process described herein or known in the art. Sequence reads can be generated from one end of nucleic acid fragments (e.g., “single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, where, for example, most of the sequence reads can be smaller than 200 bp. A sequence read can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “sequence read count” or “read count” refers to the total number of nucleic acid reads generated for each nucleic acid molecule in a subset of nucleic acid molecules, which may or may not be equivalent to the number of nucleic acid molecules generated, during a nucleic acid sequencing reaction. In some embodiments, a read count refers to a count of sequence reads in the plurality of sequence reads that map (e.g., align) to a corresponding reference sequence (e.g., complete and/or incomplete genome) for a respective predefined category (e.g., microorganism). In some embodiments, a read count refers to a count of unique sequence reads in the plurality of sequence reads that map to a corresponding reference sequence (e.g., complete and/or incomplete genome) for a respective predefined category (e.g., microorganism). In some embodiments, a read count refers to a count of sequence reads in the plurality of sequence reads that is normalized (e.g., relative to a target nucleotide sequence length for all or a portion of a corresponding reference sequence).

As used herein, the term “depth,” “read depth,” or “sequencing depth” refers to a total number of unique nucleic acid fragments encompassing a particular locus or region of the reference sequence (e.g., complete and/or incomplete genome) of a subject that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is an integer, because it represents the actual sequencing depth for a particular locus. Sequencing depth can also be applied to multiple loci, or a whole genome or reference sequence, in which case Y can refer to the mean or average number of times a locus or a haploid genome, or a whole genome or reference sequence, respectively, is sequenced. Alternatively, depth, read-depth, or sequencing depth can refer to a measure of central tendency (e.g., a mean or mode) of the number of unique nucleic acid fragments that encompass one of a plurality of loci or regions of the genome or reference sequence of a subject that are sequenced in a particular sequencing reaction. For example, in some embodiments, sequencing depth refers to the average depth of every locus across an arm of a chromosome, a targeted sequencing panel, an exome, or an entire genome or reference sequence. In such case, Y may be expressed as a fraction or a decimal, because it refers to an average depth across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “coverage” refers to the proportion of a reference sequence (e.g., a complete and/or incomplete reference genome) that is covered by mapped (e.g., aligned) sequence reads. In some embodiments, coverage is a percent coverage of the mapping of a plurality of sequence reads against the respective reference sequence. For instance, in some embodiments, if after mapping of a plurality of sequence reads to a reference sequence, 90% of the reference sequence is covered by mapped (e.g., aligned) reads, then the coverage is 90%.

As used herein, the terms “genome” or “reference genome” refer to any particular known, sequenced or characterized genome, whether partial or complete, of any predefined category (e.g., organism, microorganism, and/or virus) that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of a predefined category (e.g., organism, microorganism, and/or virus), expressed in nucleic acid sequences. As used herein, a reference sequence or reference genome often is an assembled or partially assembled genomic sequence from a representative member of a predefined category (e.g., an individual) or from multiple representatives of a predefined category (e.g., multiple individuals). In some embodiments, a reference genome is an assembled or partially assembled genomic sequence from one or more human individuals. In some embodiments, a reference genome is an assembled or partially assembled genomic sequence from one or more microorganisms of the same species. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

In some embodiments, a genome is a complete genome. In some embodiments, a genome is an incomplete genome. For example, in some embodiments, an incomplete genome is at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the complete genome.

In some embodiments, a complete or incomplete genome is less than 1 megabase pairs (Mb), less than 0.5 Mb, less than 0.4 Mb, less than 0.3 Mb, less than 0.2 Mb, or less than 0.1 Mb. In some embodiments, a complete or incomplete genome is at least 1 Mb, at least 2 Mb, at least 3 Mb, at least 4 Mb, at least 5 Mb, at least 6 Mb, at least 7 Mb, at least 8 Mb, at least 9 Mb, at least 10 Mb, at least 15 Mb, at least 20 Mb, at least 25 Mb, at least 30 Mb, at least 35 Mb, at least 40 Mb, at least 45 Mb, at least 50 Mb, at least 100 Mb, at least 200 Mb, at least 500 Mb, at least 1,000 Mb, at least 2,000 Mb, at least 3,000 Mb, at least 4,000 Mb, at least 5,000 Mb, at least 10 gigabase pairs (Gb), at least 20 Gb, or at least 50 Gb.

In some embodiments, a complete or incomplete genome spans a region of a reference genome comprising at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, at least 2,000, at least 3,000, at least 4,000, at least 5,000, at least 10,000, or at least 50,000 genes. In some embodiments, a complete or incomplete genome spans a region of a reference genome comprising between 1 and 10, between 10 and 50, between 50 and 100, between 100 and 500, between 500 and 1000, between 1000 and 2000, between 2000 and 5000, between 5000 and 10,000, between 10,000 and 50,000, or more than 50,000 genes.

In some embodiments, a complete or incomplete genome spans a region of a reference genome comprising at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, or at least 500 antimicrobial resistance markers. In some embodiments, a complete or incomplete genome spans a region of a reference genome comprising between 1 and 10, between 10 and 50, between 50 and 100, or more than 100 antimicrobial resistance markers.

In some embodiments, a complete or incomplete genome is obtained from one or more nucleotide sequence databases and/or microorganism databases, including but not limited to NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. See, for example, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gky1054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19; each of which is hereby incorporated by reference herein in its entirety.

As used herein, the term “reference sequence” refers to a sequence of nucleotide bases. In some embodiments, a reference sequence is a reference genome. In some embodiments, a reference sequence is a complete or incomplete genome. In some embodiments, a reference sequence is less than 1 megabase pairs (Mb), less than 0.5 Mb, less than 0.4 Mb, less than 0.3 Mb, less than 0.2 Mb, or less than 0.1 Mb in length. In some embodiments, a reference sequence is at least 1 Mb, at least 2 Mb, at least 3 Mb, at least 4 Mb, at least 5 Mb, at least 6 Mb, at least 7 Mb, at least 8 Mb, at least 9 Mb, at least 10 Mb, at least 15 Mb, at least 20 Mb, at least 25 Mb, at least 30 Mb, at least 35 Mb, at least 40 Mb, at least 45 Mb, at least 50 Mb, at least 100 Mb, at least 200 Mb, at least 500 Mb, at least 1,000 Mb, at least 2,000 Mb, at least 3,000 Mb, at least 4,000 Mb, at least 5,000 Mb, at least 10 gigabase pairs (Gb), at least 20 Gb, or at least 50 Gb in length. In some embodiments, a reference sequence length is between 0.2 Mb and 1 Mb in length. In some embodiments, a reference sequence length is between 0.4 Mb and 2 Mb in length. In some embodiments, a reference sequence length is between 100 Kb and 1 Mb in length

In some embodiments, a reference sequence spans a region of a reference genome comprising at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, at least 2,000, at least 3,000, at least 4,000, at least 5,000, at least 10,000, or at least 50,000 genes. In some embodiments, a reference sequence spans a region of a reference genome comprising between 1 and 10, between 10 and 50, between 50 and 100, between 100 and 500, between 500 and 1000, between 1000 and 2000, between 2000 and 5000, between 5000 and 10,000, between 10,000 and 50,000, or more than 50,000 genes.

In some embodiments, a reference sequence comprises at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, or at least 500 antimicrobial resistance markers. In some embodiments, a reference sequence consists of between 1 and 10, between 10 and 50, between 50 and 100, or more than 100 antimicrobial resistance markers.

The implementations described herein provide various technical solutions for quantification of predefined categories (e.g., microorganisms) in a sequencing dataset obtained from a sequencing reaction of nucleic acids from a biological sample. Examples of such sequencing datasets include those arising from sample processing and/or sequencing as disclosed in U.S. Patent Application No. 62/696,783, entitled “Methods and Systems for Processing Samples,” filed Jul. 11, 2018, and PCT Application No. PCT/US2019/060915, entitled “Directional Targeted Sequencing,” filed Nov. 12, 2019, each of which is hereby incorporated by reference. Details of implementations are now described in conjunction with the Figures.

Exemplary System Embodiments

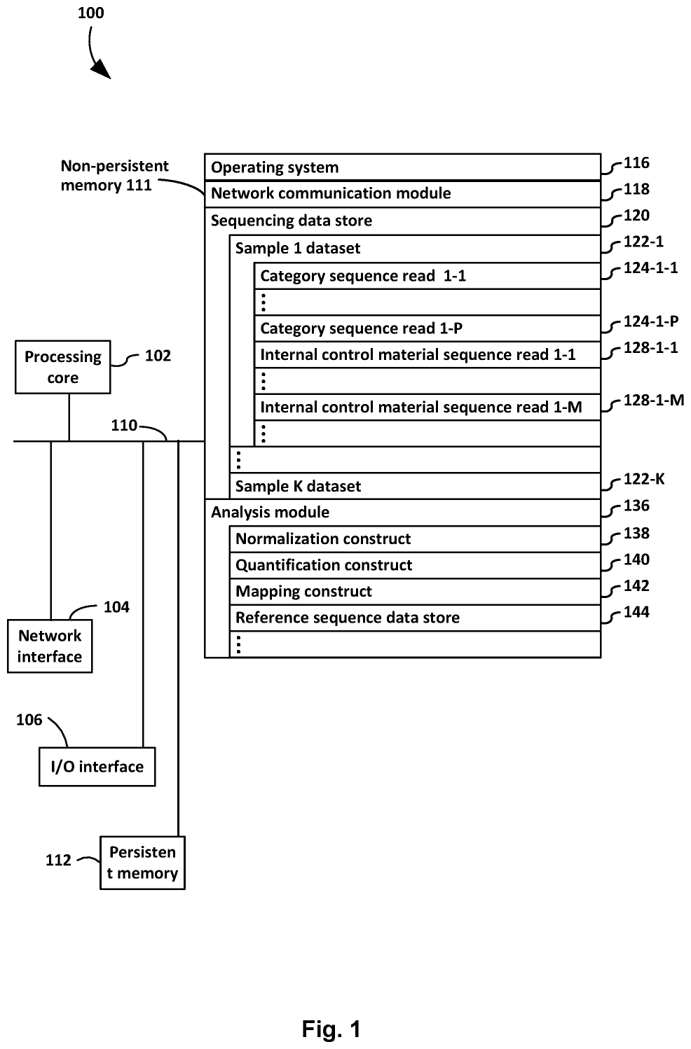

FIG. 1 is a block diagram illustrating a system 100 for determining an amount of a predefined category represented in a sample, in accordance with some implementations. The device 100 in some implementations includes one or more central processing units (CPU(s)) 102 (also referred to as processors), one or more network interfaces 104, a user interface 106, a non-persistent memory 111, a persistent memory 112, and one or more communication buses 110 for interconnecting these components. The one or more communication buses 110 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprises non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the visualization system 100 with other devices, or a communication network;
a sequencing data store 120 obtained from a sequencing of the sample 122 (e.g., 122-1, . . . , 122-K) and an added known quantity of an internal control material, comprising a first plurality of sequence reads 124 corresponding to one or more nucleic acid molecules originating from the predefined category (e.g., 124-1-1, . . . , 124-1-P) and a second plurality of sequence reads 128 corresponding to one or more nucleic acid molecules originating from the internal control material (e.g., 128-1-1, . . . , 128-1-M);
an analysis module 136 comprising a normalization construct 138 and a quantification construct 140 for determining, from the first plurality of sequence reads 124, a first read count for the number of sequence reads originating from the predefined category, where the first read count is normalized based on a first target nucleotide sequence length, determining, from the second plurality of sequence reads 128, a second read count for the number of sequence reads originating from the internal control material, where the second read count is normalized based on a second target nucleotide sequence length, and calculating the amount of the predefined category in the sample based on the first read count, the second read count, and the known quantity of the internal control material;
optionally, a mapping construct 142 for mapping the plurality of sequence reads against one or more reference sequences; and
optionally, a reference sequence data store 144 comprising a plurality of reference sequences corresponding to one or more predefined categories.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 may retrieve all or a portion of such data when needed.

Although FIG. 1 depicts a “system 100,” the figures are intended more as a functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

### Specific Embodiments of the Disclosure

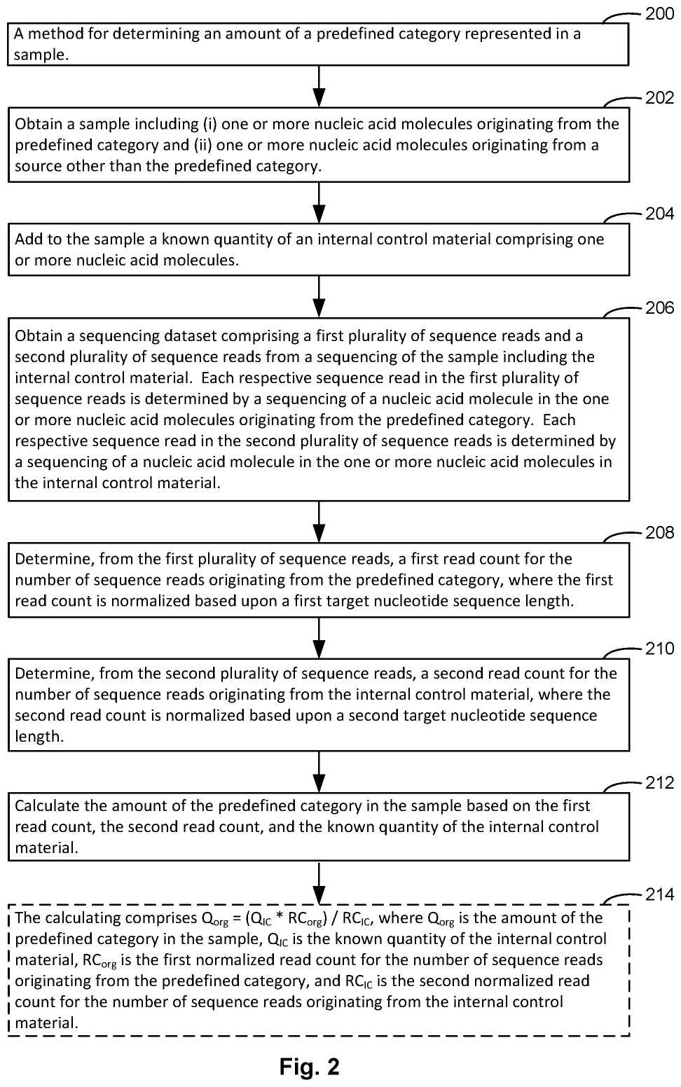

While a system in accordance with the present disclosure has been disclosed with reference to FIG. 1, a method in accordance with the present disclosure is now detailed with reference to FIG. 2. In some embodiments, the presently disclosed systems and methods are used in conjunction with the systems and methods described in, for example, IDbyDNA, 2019, “Explify Software v1.5.0 User Manual,” Document No. TH-2019-200-006, pp. 1-44, which is hereby incorporated by reference herein in its entirety for all purposes.

Referring to Block 200, the present disclosure provides a method for determining an amount (e.g., a concentration) of a first predefined category (e.g., a microorganism) in a sample.

In some embodiments, the method disclosed herein is used to determine an amount of a predefined category represented in a sample, where the predefined category is present in the sample at a concentration of between 0 and 10¹³ copies/mL, between 10² and 10⁷ copies/mL, or between 10⁴ and 10⁶ copies/mL. In some embodiments, the method is used to determine an amount of a predefined category represented in a sample, where the predefined category is present in the sample at a concentration of no more than 10¹⁰ copies/mL, no more than 10⁷ copies/mL, no more than 10⁶ copies/mL, no more than 10⁵ copies/mL, no more than 10⁴ copies/mL, no more than 1000 copies/mL, no more than 100 copies/mL, no more than 10 copies/mL, or less. In some embodiments, the method is used to determine an amount of a predefined category represented in a sample, where the predefined category is present in the sample at a concentration of at least 1 copy/mL, at least 10 copies/mL, at least 100 copies/mL, at least 1000 copies/mL, at least 10⁴ copies/mL, at least 10⁵ copies/mL, at least 10⁶ copies/mL, at least 10⁷ copies/mL, at least 10⁸ copies/mL, at least 10⁹ copies/mL, at least 10¹⁰ copies/mL, or more.

In some embodiments, the first predefined category is an organism. In some embodiments, the first predefined category is a microorganism. In some embodiments, the first predefined category is any entity that can be represented by nucleic acid molecules in a sample, such as a cell, an organism, a microorganism, a tissue type, a cell type, and/or a tissue or cell origin. In some embodiments, the first predefined category is any number or size of a respective entity, such as a population of cells, a population of organisms, a population of microorganisms, a tissue, and/or an organ. In some embodiments, the first predefined category is a classification of a respective entity, such as a characteristic of a cell or cells that can be determined using nucleic acid molecules. For example, in some embodiments, the first predefined category is a cancer condition, such as a presence or absence of cancer, a cancer stage, a cancer type, a tissue of origin, and/or a metastatic status (e.g., where the source other than the first predefined category is an individual organism). In another example, the first predefined category is a population of cancer cells. In some embodiments, the first predefined category is a tumor. In some embodiments, the first predefined category is a fetus (e.g., where the source other than the first predefined category is a pregnant individual). In some embodiments, the first predefined category is a population of activated cells (e.g., lymphocytes), cells undergoing a biological process (e.g., cell division, differentiation, activation of functional pathways, etc.), and/or cells undergoing a treatment (e.g., a chemical, biological and/or radiological treatment),In some embodiments, the first predefined category is a first population of biological material normally present in a sample (e.g., a sub-population of endogenous cells in an individual) and the source other than the first predefined category includes all other biological materials originating from the sample (e.g., all other cells in the individual) that are distinct from the first population of biological material. In some embodiments, the first predefined category is a first population of biological material that is not normally present in a sample (e.g., infecting and/or contaminating microorganisms in a sample and/or an individual) and the source other than the first predefined category includes any one or more biological materials that are normally present in the sample (e.g., endogenous cells in the sample and/or individual).

In some embodiments, the predefined category is selected from a plurality of predefined categories. In some embodiments, the plurality of predefined categories consists of two, three, four, five, six, seven, eight, nine, ten, eleven, twelve, thirteen, fourteen, fifteen, sixteen, seventeen, eighteen, nineteen or twenty categories. In some embodiments, the plurality of predefined categories consists of between two and twenty thousand categories. In some embodiments, the plurality of categories comprises 5 or more, 10 or more, 15 or more, 20 or more, 100 or more, 1000 or more or 10,000 or more categories. In some such embodiments, each respective predefined category in the plurality of predefined categories is an organism. In some embodiments, each respective predefined category in the plurality of predefined categories is a microorganism. In some embodiments, each respective predefined category in the plurality of predefined categories is any entity that can be represented by nucleic acid molecules in a sample, such as a cell, an organism, a microorganism, a tissue type, a cell type, and/or a tissue or cell origin. In some embodiments, each respective predefined category in the plurality of predefined categories is any number or size of a respective entity, such as a population of cells, a population of organisms, a population of microorganisms, a tissue, and/or an organ. In some embodiments, each respective predefined category in the plurality of predefined categories is a classification of a respective entity, such as a characteristic of a cell or cells that can be determined using nucleic acid molecules. For example, in some embodiments, a respective predefined category is a cancer condition, such as a presence or absence of cancer, a cancer stage, a cancer type, a tissue of origin, and/or a metastatic status (e.g., where the source other than the first predefined category is an individual organism). In another example, in some embodiments, a respective predefined category is a population of cancer cells. In some embodiments, a respective predefined category is a tumor. In some embodiments, a respective predefined category is a fetus (e.g., where the source other than the first predefined category is a pregnant individual). In some embodiments, a respective predefined category is a population of activated cells (e.g., lymphocytes), cells undergoing a biological process (e.g., cell division, differentiation, activation of functional pathways, etc.), and/or cells undergoing a treatment (e.g., a chemical, biological and/or radiological treatment).

In some embodiments, a respective predefined category is a first population of biological material normally present in a sample (e.g., a sub-population of endogenous cells in an individual) and the source other than the respective predefined category includes all other biological materials originating from the sample (e.g., all other cells in the individual) that are distinct from the first population of biological material. In some embodiments, a respective predefined category is a first population of biological material that is not normally present in a sample (e.g., infecting and/or contaminating microorganisms in a sample and/or an individual) and the source other than the respective predefined category includes any one or more biological materials that are normally present in the sample (e.g., endogenous cells in the sample and/or individual).

Any embodiment for a first predefined category disclosed herein, such as those described above and in the following sections, are applicable to any other respective predefined category referred to herein, including any second, third, fourth, or subsequent predefined category in one or more samples. Moreover, any embodiment for a respective predefined category disclosed herein is further contemplated as including any substitutions, modifications, additions, deletions, and/or combinations thereof, as will be apparent to one skilled in the art.

In some embodiments, the method disclosed herein is used to determine an amount of one or more predefined categories represented in a sample, where the sample comprises two or more taxonomically distinct populations of predefined categories (e.g., distinct taxa in a community of multiple microbial populations). For example, in some instances, a taxonomically distinct predefined category is a species, subspecies, strain, and/or mutant (e.g., of an organism).

In some embodiments, the method disclosed herein is used to determine an amount of a first predefined category in a plurality of predefined categories (e.g., taxa), where the first predefined category consists of less than 1 in 10, less than 1 in 100, less than 1 in 1000, less than 1 in 10⁴, less than 1 in 10⁵, less than 1 in 10⁶, less than 1 in 10⁷, less than 1 in 10⁸, or less than 1 in 10⁹ of the total predefined categories in the plurality of predefined categories. In some embodiments, the method disclosed herein is used to determine an amount of a first predefined category in a plurality of predefined categories (e.g., taxa), where the first predefined category consists from between than 1 in 10 and less than 1 in 10⁹ of the total predefined categories in the plurality of predefined categories. In some embodiments, the method disclosed herein is used to determine an amount of a first predefined category in a plurality of predefined categories (e.g., taxa), where the first predefined category consists from between than 1 in 100 and less than 1 in 10⁸ of the total predefined categories in the plurality of predefined categories. In some embodiments, the method disclosed herein is used to determine an amount of a first predefined category in a plurality of predefined categories (e.g., taxa), where the first predefined category consists from between than 1 in 1000 and less than 1 in 10⁷ of the total predefined categories in the plurality of predefined categories. In some embodiments, the method disclosed herein is used to determine an amount of a first predefined category in a plurality of predefined categories (e.g., taxa), where the first predefined category consists from between than 1 in 10,000 and less than 1 in 10⁶ of the total predefined categories in the plurality of predefined categories. In some embodiments, the method disclosed herein is used to determine an amount of a first predefined category in a plurality of predefined categories, where the first predefined category consists of less than 60%, less than 50%, less than 40%, less than 30%, less than 20%, less than 10%, less than 5%, less than 1%, less than 0.5%, less than 0.1%, less than 0.05%, less than 0.01%, or less than 0.001% of the total population of predefined categories in the plurality of predefined categories.

For example, in some embodiments, a plurality of predefined categories comprises a community of microorganisms, such as an environmental and/or clinical sample (e.g., a microbiome). In some embodiments, the method is used to determine an amount of a majority and/or a minority population of microorganisms in a sample. In some embodiments, the method is used to determine an amount of a microorganism that is present at a low concentration (e.g., less than 50%, less than 40%, less than 20%, less than 10%, less than 5%, or less than 1%) within a community of microorganisms. In some embodiments, the plurality of predefined categories comprises a first predefined category of interest (e.g., a first microorganism for quantification) and one or more predefined categories other than the first predefined category (e.g., a co-infecting and/or contaminating microorganism).

Subjects and Samples.

Referring to Block 202, the method comprises obtaining a sample including (i) one or more nucleic acid molecules originating from the first predefined category and (ii) one or more nucleic acid molecules originating from a source other than the first predefined category.

In some embodiments, the sample is obtained from a biological subject. For example, in some embodiments, the subject is a human (e.g., a patient). In some embodiments, the sample is obtained from any tissue, organ or fluid from the subject. In some embodiments, a plurality of samples is obtained from the subject (e.g., a plurality of replicates and/or a plurality of samples including a healthy sample and a diseased sample).

In some embodiments, the sample is obtained from a human with a disease condition (e.g., an infectious disease and/or a disease caused by a pathogenic microorganism). In some embodiments, the disease condition is influenza, common cold, measles, rubella, chickenpox, norovirus, polio, infectious mononucleosis (mono), herpes simplex virus (HSV), human papillomavirus (HPV), human immunodeficiency virus (HIV), viral hepatitis (e.g., hepatitis A, B, C, D, and/or E), viral meningitis, West Nile Virus, rabies, ebola, strep throat, bacterial urinary tract infections (UTIs) (e.g., coliform bacteria), bacterial food poisoning (e.g., E. coli, Salmonella, and/or Shigella), bacterial cellulitis (e.g., Staphylococcus aureus (MRSA)), bacterial vaginosis, gonorrhea, chlamydia, syphilis, Clostridium difficile (C. difficile), tuberculosis, whooping cough, pneumococcal pneumonia, bacterial meningitis, Lyme disease, cholera, botulism, tetanus, anthrax, vaginal yeast infection, ringworm, athlete's foot, thrush, aspergillosis, histoplasmosis, Cryptococcus infection, fungal meningitis, malaria, toxoplasmosis, trichomoniasis, giardiasis, tapeworm infection, roundworm infection, pubic and head lice, scabies, leishmaniasis, and/or river blindness. In some embodiments, the sample is obtained from a human with a viral respiratory disease. In some embodiments, the sample is obtained from a human with a coronavirus infection. In some embodiments, the biological sample is obtained from a human with a SARS-CoV-2 infection.

In some embodiments, the disease condition is a cancer. In some embodiments, the cancer is ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and/or papillary renal cell carcinoma.

In some embodiments, the sample is obtained from a pregnant individual. In some embodiments, the sample is obtained from a pregnant human.

In some embodiments, the sample is a clinical sample, a diagnostic sample, an environmental sample, a consumer quality sample, a food sample, a biological product sample, a microbial testing sample, a tumor sample, a forensic sample and/or a laboratory or hospital sample. In some embodiments, biological sample is obtained from a human or an animal. In some embodiments, a biological sample is a sample from a patient undergoing a treatment.

In some embodiments, the sample is collected from an environmental source, such as a field (e.g., an agricultural field), lake, river, creek, ocean, watershed, water tank, water reservoir, pool (e.g., swimming pool), pond, air vent, wall, roof, soil, plant, and/or other environmental source. In some embodiments, the sample is collected from an industrial source, such as a clean room (e.g., in manufacturing or research facilities), hospital, medical laboratory, pharmacy, pharmaceutical compounding center, food processing area, food production area, water or waste treatment facility, and/or food product. In some embodiments, the sample is an air sample, such as ambient air in a facility (e.g., a medical facility or other facility), exhaled or expectorated air from a subject, and/or aerosols, including any biological contaminants present therein (e.g., bacteria, fungi, viruses, and/or pollens). In some embodiments, the sample is a water sample, such as dialysis systems in medical facility (e.g., to detect waterborne pathogens of clinical significance and/or to determine the quality of water in a facility). In some embodiments, the sample is an environmental surface sample, such as before or after a sterilization or disinfecting process (e.g., to confirm the effectiveness of the sterilization or disinfecting procedure).

In some embodiments, the sample is a control sample (e.g., a positive control, negative control, and/or blank control).

In some embodiments, the one or more nucleic acid molecules in the sample originating from the first predefined category is RNA or DNA. In some embodiments, the one or more nucleic acid molecules in the sample originating from the source other than the first predefined category is RNA or DNA.

In some embodiments, the sample comprises or consists essentially of RNA. In some embodiments, the sample comprises or consists essentially of DNA. In some embodiments, the one or more nucleic acid molecules are included within cells. Alternatively, or in addition, in some embodiments, the one or more nucleic acid molecules are not included within cells (e.g., cell-free nucleic acid molecules). In some embodiments, samples comprising cell-free nucleic acid molecules include samples from which cells have been removed, samples not subjected to a lysis step, and/or samples treated to separate cellular nucleic acid molecules from cell-free nucleic acid molecules. For example, in some embodiments, cell-free nucleic acid molecules include nucleic acid molecules released into circulation upon death of a cell, which can be isolated from a plasma fraction of a blood sample.

In some embodiments, the one or more nucleic acid molecules in the sample originating from the first predefined category are nucleic acid molecules originating from a first microorganism, such as a pathogenic microorganism (see, for example, “Microorganisms,” below). In some embodiments, the one or more nucleic acid molecules in the sample originating from the first predefined category originate from a first microorganism (e.g., a first microbiological taxon, such as a species, subspecies, strain, and/or mutant), and the one or more nucleic acid molecules in the sample originating from the source other than the first predefined category originate from a second microorganism (e.g., a second microbiological taxon, such as a species, subspecies, strain, and/or mutant). In some such embodiments, the sample comprises two or more distinct populations of microorganisms (e.g., a community of microbial populations).

In some embodiments, the one or more nucleic acid molecules in the sample originating from the source other than the first predefined category originate from a host subject (e.g., where the first predefined category is an infecting and/or contaminating microorganism). In some embodiments, the one or more nucleic acid molecules in the sample originating from the source other than the first predefined category originate from a human (e.g., a patient with an infectious disease).

In some embodiments, the one or more nucleic acid molecules in the sample comprise any of the embodiments described herein. See, for example, Definitions: Nucleic acids.

Other suitable embodiments of samples are as described in the above sections (see, for example, Definitions: Samples), and any substitutions, modifications, additions, deletions, and/or combinations thereof, as will be apparent to one skilled in the art.

Microorganisms.

In some embodiments, the first predefined category is a microorganism (e.g., an infecting and/or contaminating microorganism in the sample).

In some embodiments, a microorganism is a single-celled organism and/or a colony of single-celled organisms. In some embodiments, a microorganism is one or more members of a taxon (e.g., a species, subspecies, strain, mutant, and/or other taxonomic group within which one or more individual biological entity can be classified). In some embodiments, a microorganism is eukaryotic or prokaryotic. In some embodiments, a microorganism is any one of the microorganisms described herein (See, Definitions: “Microorganisms,” above). In some embodiments, a microorganism is any one of the microorganisms selected from a database, including but not limited to NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database.

In some embodiments, the first predefined category (e.g., microorganism) is a commensal organism (e.g., is commonly associated with the source or site of sample collection and/or is not considered to be harmful). For example, hundreds of microorganisms are known to co-exist in the oral microbiome, and their existence in a sample collected from the oral cavity of a subject may not be indicative of a disease state. In some embodiments, the first predefined category (e.g., microorganism) exists in a symbiotic (e.g., endosymbiotic) relationship with a subject (e.g., a host organism). In some embodiments, the first predefined category is a microorganism that is considered healthy, normal, and/or beneficial to health, such as a probiotic. Other suitable alternatives include various microorganisms that are known or have been shown to contribute to immune health, synthesize useful vitamins, and/or ferment indigestible carbohydrates.

In some embodiments, the first predefined category (e.g., microorganism) is a pathogen (e.g., disease-causing), such as a human, animal, or plant-infective pathogen.

In some embodiments, the first predefined category is associated with a disease and/or is known or has been shown to be otherwise harmful to a population, such as a human population. For example, in some embodiments, the first predefined category is a pathogen that is a causative agent in an infectious disease. In some embodiments, the first predefined category is present in the sample (e.g., the subject, source and/or site of collection) at an asymptomatic level (e.g., at a level unlikely to induce disease or infection). In some embodiments, the first predefined category is present in the sample (e.g., the subject, source and/or site of collection) at a symptomatic level (e.g., a chronic and/or acute symptomatic level).

In some embodiments, the first predefined category is associated with and/or the causative agent of, for example, a brain infection, urinary tract disease, respiratory disease, CNS, and/or cancer. In some embodiments, the first predefined category is associated with and/or the causative agent of influenza, common cold, measles, rubella, chickenpox, norovirus, polio, infectious mononucleosis (mono), herpes simplex virus (HSV), human papillomavirus (HPV), human immunodeficiency virus (HIV), viral hepatitis (e.g., hepatitis A, B, C, D, and/or E), viral meningitis, West Nile Virus, rabies, Ebola, strep throat, bacterial urinary tract infections (UTIs) (e.g., coliform bacteria), bacterial food poisoning (e.g., E. coli, Salmonella, and/or Shigella), bacterial cellulitis (e.g., Staphylococcus aureus (MRSA)), bacterial vaginosis, gonorrhea, chlamydia, syphilis, Clostridium difficile (C. diff), tuberculosis, whooping cough, pneumococcal pneumonia, bacterial meningitis, Lyme disease, cholera, botulism, tetanus, anthrax, vaginal yeast infection, ringworm, athlete's foot, thrush, aspergillosis, histoplasmosis, Cryptococcus infection, fungal meningitis, malaria, toxoplasmosis, trichomoniasis, giardiasis, tapeworm infection, roundworm infection, pubic and head lice, scabies, leishmaniasis, and/or river blindness.

In some embodiments, the first predefined category is associated with and/or the causative agent of a viral respiratory disease. In some embodiments, the first predefined category is associated with and/or the causative agent of a coronavirus infection. In some embodiments, the first predefined category is associated with and/or the causative agent of a SARS-CoV-2 infection.

In some embodiments, the first predefined category (e.g., microorganism) is selected from the group consisting of bacterial, fungal, viral, and parasitic.

For instance, in some embodiments, the first predefined category is selected from viruses, bacteria, protists, helminths, monerans, chromalveolata, archaea, and/or fungi. Non-limiting examples of viruses include Human Immunodeficiency Virus, Ebola virus, rhinovirus, influenza, rotavirus, hepatitis virus, West Nile virus, ringspot virus, mosaic viruses, herpesviruses, and/or lettuce big-vein associated virus. Non-limiting examples of bacteria include Staphylococcus aureus, Staphylococcus aureus Mu3, Staphylococcus epidermidis, Streptococcus agalactiae, Streptococcus pyogenes, Streptococcus pneumonia, Escherichia coli, Citrobacter koseri, Clostridium perfringens, Enterococcus faecalis, Klebsiella pneumonia, Lactobacillus acidophilus, Listeria monocytogenes, Propionibacterium granulosum, Pseudomonas aeruginosa, Serratia marcescens, Bacillus cereus, Staphylococcus aureus Mu50, Yersinia enterocolitica, Staphylococcus simulans, Micrococcus luteus, and/or Enterobacter aerogenes. Non-limiting examples of fungi include Absidia corymbifera, Aspergillus niger, Candida albicans, Geotrichum candidum, Hansenula anomala, Microsporum gypseum, Monilia, Mucor, Penicilliusidia corymbifera, Aspergillus niger, Candida albicans, Geotrichum candidum, Hansenula anomala, Microsporum gypseum, Monilia, Mucor, Penicillium expansum, Rhizopus, Rhodotorula, Saccharomyces bayabus, Saccharomyces carlsbergensis, Saccharomyces uvarum, and/or Saccharomyces cerevisiae.

In some embodiments, the first predefined category is a coronavirus. In some embodiments, the predefined category is severe acute respiratory syndrome coronavirus (e.g., SARS-CoV-2). In some embodiments, the predefined category is an influenza virus. In some embodiments, the predefined category is an influenza A virus.

In some embodiments, the first predefined category is a microorganism in a plurality of microorganisms (e.g., in a community of microorganisms).

For example, in some embodiments, the first predefined category is a microorganism in a plurality of microorganisms comprising at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 microorganisms (e.g., taxa). In some embodiments, the first predefined category is a microorganism in a plurality of microorganisms comprising at least 100, at least 200, at least 300, at least 400, at least 500, at least 600, at least 700, at least 800, at least 900, at least 1000, at least 2000, at least 3000, at least 4000, at least 5000, at least 10,000 or at least 50,000 microorganisms (e.g., taxa). In some embodiments, the first predefined category is a microorganism in a plurality of microorganisms comprising between 1 and 50, between 50 and 100, between 100 and 200, between 200 and 500, between 500 and 1000, between 1000 and 2000, between 2000 and 3000, between 3000 and 5000, between 5000 and 10,000, between 10,000 and 50,000, or more than 50,000 microorganisms (e.g., taxa). In some embodiments, the first predefined category is a microorganism in a plurality of microorganisms comprising no more than 10,000, no more than 5,000, no more than 3000, no more than 2000, no more than 1000, no more than 500, no more than 300, no more than 200, no more than 100, no more than 50, no more than 40, no more than 30, no more than 20, or no more than 10 microorganisms (e.g., taxa). In some embodiments, one or more microorganisms in the plurality of microorganisms is selected from any one or more of the lists provided herein and/or any one or more of the databases provided herein. In some embodiments, each microorganism in the plurality of microorganisms is selected from any one or more of the lists provided herein and/or any one or more of the databases provided herein.

In some embodiments, the first predefined category is associated with a corresponding reference sequence (e.g., a reference genome). In some embodiments, the corresponding reference sequence for the predefined category is obtained from a nucleotide sequence database. A nucleotide sequence database can be, for example, a global genome database or a microorganism-specific genome database. For example, in some embodiments, a reference sequence for a predefined category is obtained from NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. See, for example, Zhulin, 2015, “Databases for Microbiologists,” J Bacteriol 197:2458-2467, doi:10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gky1054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19; each of which is hereby incorporated by reference herein in its entirety.

In some embodiments, the first predefined category is associated with an antimicrobial resistance marker (e.g., an AMR gene that is determined based on an annotation and/or a platform-curated genome library).

In some embodiments, an antimicrobial resistance marker is a gene. In some embodiments, an antimicrobial resistance marker is a nucleic acid sequence obtained from a reference genome. In some embodiments, an antimicrobial resistance marker is any of the embodiments described herein (see, for example, Definitions: “Antimicrobial resistance markers”). In some embodiments, an antimicrobial resistance marker is selected from Table 1 and/or selected from one or more databases, including but not limited to the National Database of Antibiotic Resistant Organisms (NDARO), the Comprehensive Antibiotic Resistance Database (CARD), ResFinder, PointFinder, ARG-ANNOT, ARGs-OSP, PlasmoDB, the Mycology Antifungal Resistance Database (MARDy), DBDiaSNP, the HIV Drug Resistance Database, the Virus Pathogen Resource (ViPR), and/or any of the databases used for selecting one or more microorganisms, as disclosed above.

Internal Control Material.

Referring to Block 204, the method disclosed herein further comprises adding to the sample a known quantity (e.g., a concentration) of an internal control material comprising one or more nucleic acid molecules.

In some embodiments, the internal control material is added to the sample after sample collection but prior to preparation for analysis, including lysing, permeabilizing, nucleic acid extraction, nucleic acid amplification, sequencing library preparation, sequencing, and/or data analysis. In some embodiments, the internal control material is added to the sample after sample collection but prior to any laboratory handing or sample treatment, including treatment with a preservation agent, storage, freeze-thaw, and/or aliquoting). In some embodiments, the internal control material is added to the sample immediately after collection. In some embodiments, the sample is divided into a plurality of aliquots and the internal control material is added to a respective aliquot in the plurality of aliquots.

In some embodiments, the internal control material is a natural or synthetic material having the ability to mimic a target predefined category (e.g., a microorganism for quantification) and/or a portion thereof, and its behavior throughout a workflow (e.g., sample loss, extraction efficiency, and/or sequencing efficiency during sample processing, sequencing and/or analysis). In some embodiments, the internal control material comprises one or more of a similar physical structure (e.g., membrane, capsid, and/or envelope), nucleic acid sequence (e.g., target nucleotide sequence), and/or quantity (e.g., microorganism load and/or nucleic acid copies/mL) so as to exhibit similar responses as the target predefined category during sample preparation, lysis, nucleic acid extraction yield, amplification, sequencing, analysis, and/or other processing manipulations.

In some embodiments, the internal control material comprises material originating from a source that is of the same type as the first predefined category. In some embodiments, the internal control material comprises material originating from a source that is of the same type as a respective predefined category in a plurality of predefined categories. In some embodiments, the internal control material comprises a material selected based on its similarity to a target predefined category for quantification. In some embodiments, the internal control material comprises naturally occurring and/or synthetic material.

For instance, in some embodiments, the internal control material is a naturally occurring material, such as an organism and/or a biological material obtained from an organism (e.g., a microorganism, a pathogen, a cell, a nucleic acid molecule, etc.). In some embodiments, the organism is selected from any one or more of the lists provided herein and/or any one or more of the databases provided herein. In some embodiments, the internal control material comprises a naturally occurring organism selected based on its similarity to a target organism for quantification (e.g., a bacteriophage selected based on an ability to mimic viral membrane, capsid, and/or envelope structure).

In some embodiments, the internal control material comprises one or more nucleic acid molecules obtained from an predefined category (e.g., DNA and/or RNA extracted from a sample of a microorganism). For example, in some embodiments, the internal control material comprises one or more nucleic acid molecules corresponding to one or more genes from an organism. In some such embodiments, a gene in the one or more genes is selected based on a known copy number in the respective organism. In some embodiments, the internal control material is obtained from an organism via a nucleic acid amplification process (e.g., PCR) for the respective one or more genes.

In some embodiments, the internal control material comprises one or more synthetic materials, such as one or more synthetic nucleic acid molecules and/or one or more synthetic particles. In some such embodiments, the synthetic material is selected based on a similarity to a target organism for quantification (e.g., a synthetic nucleotide sequence designed based on a sequence similarity to a naturally occurring nucleotide sequence in a target organism, and/or a synthetic particle selected based on an ability to mimic viral membrane, capsid, and/or envelope structures).

In some embodiments, where the internal control material comprises naturally occurring or synthetic nucleic acid molecules, the size of a respective nucleic acid molecule in the internal control material is selected based on an expected fragment size resulting from a sample processing workflow for a sample and/or a target predefined category for quantification. In some embodiments, where the internal control material comprises naturally occurring or synthetic nucleic acid molecules, the composition (e.g., GC content, complementarity, etc.) of the nucleic acid molecules in the internal control material is selected based on a similarity to the expected composition of one or more target nucleic acid molecules in a target predefined category for quantification.

Other suitable examples for internal control materials include, but are not limited to, naturally occurring plasmids, engineered plasmids, naturally occurring linear nucleic acid fragments (e.g., RNA and/or DNA), synthesized linear nucleic acid fragments (e.g., RNA, cDNA, and/or DNA), and/or the like.

In some embodiments, the internal control material comprises a plurality of naturally occurring materials (e.g., organisms and/or biological material), where each respective material in the plurality of naturally occurring materials is obtained from a respective predefined category in a plurality of predefined categories (e.g., microorganisms, pathogens, cells, nucleic acid molecules, etc.). In some embodiments, the internal control material comprises a plurality of synthetic materials, where each respective material in the plurality of synthetic materials is selected for (e.g., synthesized for) at least one respective target predefined category in a plurality of target predefined categories for quantification.

In some embodiments, the internal control material comprises a plurality of naturally occurring and/or synthetic materials specific to (e.g., obtained from and/or selected for) at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 predefined categories. In some embodiments, the internal control material comprises a plurality of naturally occurring and/or synthetic materials specific to (e.g., obtained from and/or selected for) at least 100, at least 200, at least 300, at least 400, at least 500, at least 600, at least 700, at least 800, at least 900, at least 1000, at least 2000, at least 3000, at least 4000, at least 5000, at least 10,000 or at least 50,000 predefined categories. In some embodiments, the internal control material comprises a plurality of naturally occurring and/or synthetic materials specific to (e.g., obtained from and/or selected for) between 1 and 50, between 50 and 100, between 100 and 200, between 200 and 500, between 500 and 1000, between 1000 and 2000, between 2000 and 3000, between 3000 and 5000, between 5000 and 10,000, between 10,000 and 50,000, or more than 50,000 predefined categories. In some embodiments, the internal control material consists of a plurality of naturally occurring and/or synthetic materials specific to (e.g., obtained from and/or selected for) no more than 10,000, no more than 5,000, no more than 3000, no more than 2000, no more than 1000, no more than 500, no more than 300, no more than 200, no more than 100, no more than 50, no more than 40, no more than 30, no more than 20, or no more than 10 predefined categories. In some embodiments, each material (e.g., each predefined category, each material obtained from each respective predefined category, and/or each synthetic material selected for each respective target predefined category) is labeled for identification and post-processing separation (e.g., via sequence-specific probes labeled fluorescently, radioactively, chemiluminescently, enzymatically, or the like, as are known in the art).

For example, in some embodiments, the internal control material comprises a plurality of naturally occurring and/or synthetic materials specific to (e.g., obtained from and/or selected for) at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 microorganisms (e.g., taxa). In some embodiments, the internal control material comprises a plurality of naturally occurring and/or synthetic materials specific to (e.g., obtained from and/or selected for) at least 100, at least 200, at least 300, at least 400, at least 500, at least 600, at least 700, at least 800, at least 900, at least 1000, at least 2000, at least 3000, at least 4000, at least 5000, at least 10,000 or at least 50,000 microorganisms (e.g., taxa). In some embodiments, the internal control material consists of a plurality of naturally occurring and/or synthetic materials specific to (e.g., obtained from and/or selected for) between 1 and 50, between 50 and 100, between 100 and 200, between 200 and 500, between 500 and 1000, between 1000 and 2000, between 2000 and 3000, between 3000 and 5000, between 5000 and 10,000, between 10,000 and 50,000, or more than 50,000 microorganisms (e.g., taxa). In some embodiments, the internal control material consists of a plurality of naturally occurring and/or synthetic materials specific to (e.g., obtained from and/or selected for) no more than 10,000, no more than 5,000, no more than 3000, no more than 2000, no more than 1000, no more than 500, no more than 300, no more than 200, no more than 100, no more than 50, no more than 40, no more than 30, no more than 20, or no more than 10 microorganisms (e.g., taxa). In some embodiments, each material (e.g., each microorganism, each biological material obtained from each respective microorganism, and/or each synthetic material selected for each respective target microorganism) is labeled for identification and post-processing separation (e.g., via sequence-specific probes labeled fluorescently, radioactively, chemiluminescently, enzymatically, or the like, as are known in the art).

In some embodiments, the known quantity of the internal control material is expressed as a genomic and/or transcriptomic concentration. In some embodiments, the known quantity of the internal control material is a concentration by volume and/or by weight. For example, the suitable units for the known quantity of the internal control material include, but are not limited to, copies/mL, genomic equivalents (GE)/mL, International Unit (IU)/mL, and/or copies/weight (g).

In some embodiments, the known quantity of the internal control material is between 0 and 10¹³ copies/mL, between 10² and 10⁷ copies/mL, or between 10⁴ and 10⁶ copies/mL. In some embodiments, the known quantity of the internal control material is at least 1 copy/mL, at least 10 copies/mL, at least 100 copies/mL, at least 1000 copies/mL, at least 10⁴ copies/mL, at least 10⁵ copies/mL, at least 10⁶ copies/mL, at least 10⁷ copies/mL, at least 10⁸ copies/mL, at least 10⁹ copies/mL, at least 10¹⁰ copies/mL, or more. In some embodiments, the known quantity of the internal control material is no more than 10¹⁰ copies/mL, no more than 10⁷ copies/mL, no more than 10⁶ copies/mL, no more than 10⁵ copies/mL, no more than 10⁴ copies/mL, no more than 1000 copies/mL, no more than 100 copies/mL, no more than 10 copies/mL, or less.

In some embodiments, the known quantity of the internal control material is determined based on the linear range of the assay. For example, in some embodiments, the known quantity of the internal control material is a concentration that is above the lower limit of detection and/or below the maximum concentration expected for the assay (e.g., the maximum concentration expected for the sample, the predefined category of interest, and/or the source other than the predefined category).

Further suitable embodiments of internal control materials are described in, for example, International Application Publication No. WO2019/204588A1, entitled “Methods for Normalization and Quantification of Sequencing Data,” filed Apr. 18, 2019, the contents of which are hereby incorporated herein by reference in its entirety, as well as any substitutions, additions, deletions, modifications, and/or combinations thereof, as will be apparent to one skilled in the art.

Sequencing and Sequencing Datasets.

Referring to Block 206, the method disclosed herein further comprises obtaining, in electronic form, a sequencing dataset comprising a first plurality of sequence reads and a second plurality of sequence reads from a sequencing of the sample including the internal control material. Each respective sequence read in the first plurality of sequence reads is determined by a sequencing of a nucleic acid molecule in the one or more nucleic acid molecules originating from the first predefined category, and each respective sequence read in the second plurality of sequence reads is determined by a sequencing of a nucleic acid molecule in the one or more nucleic acid molecules originating from the internal control material.

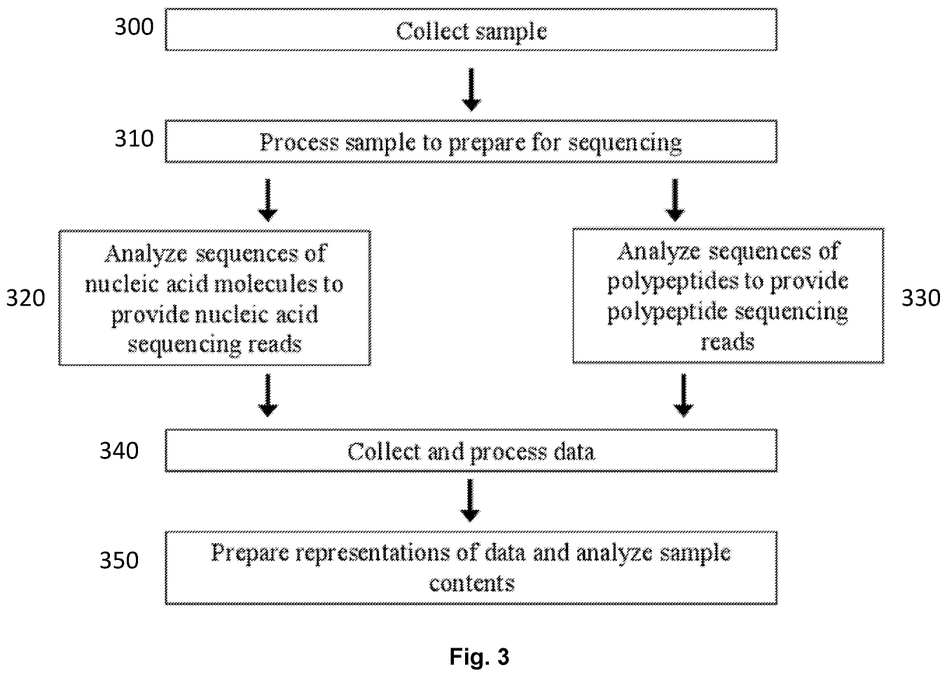

In some embodiments, a sample (e.g., a biological sample including the internal control material) is collected, prepared, sequenced (e.g., by next-generation sequencing), and/or mapped (e.g., aligned) to one or more reference sequences (e.g., complete and/or incomplete genomes) prior to quantification of a predefined category represented in the sample. In some embodiments, sample and/or internal control material processing is performed using any of the methods as disclosed in U.S. Patent Application No. 62/696,783, entitled “Methods and Systems for Processing Samples,” filed Jul. 11, 2018, which is hereby incorporated by reference herein in its entirety. As an illustrative example, in some embodiments, sample processing is performed using the method described in Example 2 and FIG. 3 (see Examples, below).

In some embodiments, the sample (e.g., including the internal control material) is contacted with a medium to preserve or enhance one or more predefined categories (e.g., microorganisms) included therein and/or to facilitate its collection. For example, in some embodiments, a sample (e.g., including the internal control material) is contacted with peptone or buffered peptone water, phosphate buffered saline, sodium chloride, ringer solution (e.g., Calgon ringer or thiosulfate ringer solutions), tryptic soy broth, brain-heart infusion broth, and/or another material. In some embodiments, a sample (e.g., including the internal control material) is subjected to elution, agitation, ultrasonic bath, centrifugation, or other processing to remove material from a sampling device and break up any clumps (e.g., clumps of cells, tissues, and/or organisms) that may be included therein.

In some embodiments, the sample (e.g., including the internal control material) is prepared for analysis by lysing or permeabilizing cells (e.g., by contacting a sample with a lysing or permeabilizing agent), degrading tissues, and/or denaturing proteins and nucleic acid molecules (e.g., by contacting a sample with a denaturing agent such as a detergent). In some embodiments, preparation of the sample (e.g., including the internal control material) also comprises releasing nucleic acid molecules from within samples. For example, in some implementations, sample preparation includes contacting the sample (e.g., including the internal control material) with an agent configured to degrade a lipid envelope and/or protein coat (e.g., capsid) of a virus to provide access to genetic material therein. In some embodiments, the sample, with or without the internal control material, is divided prior to such preparation to provide a first aliquot and a second aliquot, which first and second aliquots may undergo parallel but different processing. For example, in some instances, the first aliquot is processed to extract and preserve RNA, while the second aliquot is processed to extract and preserve DNA.

In some embodiments, the sample (e.g., including the internal control material), and/or a portion thereof, is further processed to prepare one or more nucleic acid molecules therein for analysis by nucleic acid sequencing. In some embodiments, the processing comprises extraction of the one or more nucleic acid molecules from the sample (e.g., including the internal control material).

A variety of methods are suitable for use in order to extract and/or purify nucleic acid molecules from a sample. For example, in some embodiments, nucleic acids are purified using an organic extraction method. Other non-limiting examples of extraction techniques include organic extraction followed by ethanol precipitation (e.g., using a phenol/chloroform organic reagent with or without the use of an automated nucleic acid extractor, e.g., the Model 341 DNA Extractor available from Applied Biosystems (Foster City, Calif)), stationary phase adsorption methods, and/or salt-induced nucleic acid precipitation methods, such as precipitation methods being typically referred to as “salting-out” methods. Another example of nucleic acid isolation and/or purification includes the use of magnetic particles to which nucleic acids can specifically or non-specifically bind, followed by isolation of the beads using a magnet, washing, and eluting the nucleic acids from the beads. In some embodiments, an isolation method is preceded by an enzyme digestion step to help eliminate unwanted protein from the sample, such as digestion with proteinase K and/or other like proteases. In some embodiments, nucleic acid extraction is performed using RNase inhibitors added to a lysis buffer. In some embodiments, such as for certain cell or sample types, nucleic acid extraction includes a protein denaturation and/or digestion step. In some embodiments, nucleic acid purification methods are used to isolate DNA, RNA, or both. When both DNA and RNA are isolated together during or subsequent to an extraction procedure, further steps can be employed to purify one or both separately from the other. Sub-fractions of extracted nucleic acids can also be generated, such as, for example, purification by size, sequence, and/or other physical or chemical methods.

In some embodiments, one or more nucleic acid molecules in the sample (e.g., including the internal control material) are amplified prior to sequencing. Amplification can be used to increase the detectable population of one or more nucleic acid molecules within the sample and/or the internal control material. In some embodiments, the one or more nucleic acid molecules in the sample (e.g., including the internal control material) are not amplified prior to undergoing sequencing.

Non-limiting examples of nucleic acid amplification methods include reverse transcription, primer extension, polymerase chain reaction (PCR), ligase chain reaction (LCR), helicase-dependent amplification, bridge amplification, template walking/wildfire amplification, nanoball-based amplification, asymmetric amplification, rolling circle amplification, and/or multiple displacement amplification (MDA). In some embodiments, where PCR is used, suitable non-limiting examples include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and/or touchdown PCR.

In some embodiments, preparation of the sample (e.g., including the internal control material) comprises contacting one or more nucleic acid molecules in the sample and/or the internal control material with one or more adapters and/or primers to prepare nucleic acid molecules for an amplification and/or sequencing process. In some embodiments, preparation of the sample (e.g., including the internal control material) comprises introducing primer binding sites and sample-specific identification sequences into regions of one or more nucleic acid molecules to be sequenced. In some embodiments, preparation of the sample (e.g., including the internal control material) comprises fragmenting one or more nucleic acid molecules in the sample and/or the internal control material. For example, in some instances, preparation of the sample and/or the internal control material comprises amplifying one or more nucleic acid molecules in an amplification reaction using target-specific primers that include sequencing primer binding sites and sample-specific identification sequences, such as primers with dual-indexed sequencing overhangs. In some instances, preparation of the sample and/or the internal control material comprises fragmenting the one or more nucleic acid molecules and ligating to the nucleic acid fragments sequencing-specific adapters that include sequencing primer binding sites and sample-specific identification sequences.

In some embodiments, preparation of the sample (e.g., including the internal control material) comprises preparing a sequencing library from one or more nucleic acid molecules in the sample (e.g., including the internal control material).

Additional suitable methods and embodiments for preparation of the sample and/or the internal control material are possible, as described in, for example, International Application Publication No. WO2019/204588A1, entitled “Methods for Normalization and Quantification of Sequencing Data,” filed Apr. 18, 2019, the contents of which are hereby incorporated herein by reference in its entirety.

Different types of nucleic acid molecules may undergo the same or different processing and sequencing. For example, in some embodiments, DNA molecules undergo a first sequencing process and RNA molecules undergo a second sequencing process, where the first and second sequencing processes include at least one process difference. In an example, genomic DNA such as accessible chromatin is processed according to a first sequencing method (e.g., using an assay for transposase-accessible chromatin using sequencing (ATAC-seq) method) while RNA molecules are processed according to a second sequencing method (e.g., a sequencing method that targets RNA molecules that include a polyA sequence, such as messenger RNA (mRNA) molecules). In some embodiments, different sequencing procedures are performed on the same or different samples. For example, in some embodiments, a first sequencing method to analyze a first type of nucleic acid molecule and a second sequencing method to analyze a second type of nucleic acid molecule, where the first and second sequencing methods are different and the first and second types of nucleic acid molecules are different, are performed on a same sample (e.g., at the same or different times). Alternatively or in addition, in some embodiments, a first sequencing method to analyze a first type of nucleic acid molecule is performed using a first sample and a second sequencing method to analyze a second type of nucleic acid molecule is performed using a second sample, where the first and second sequencing methods are different, the first and second types of nucleic acid molecules are different, and the first and second samples are different. In some embodiments, the first and second samples are aliquots of a single parent sample.

In some embodiments, the sequencing is quantitative or approximately quantitative. Alternatively, in some embodiments, nucleic acid sequencing is qualitative and does not provide significant insight into the relative amounts of different nucleic acid molecules included within a sample.

Various sequencing schemes can be employed. For example, in some embodiments, the sequencing is sequencing by synthesis, sequencing by hybridization, sequencing by ligation, nanopore sequencing, sequencing using nucleic acid nanoballs, pyrosequencing, single molecule sequencing (e.g., single molecule real time sequencing), single cell/entity sequencing, massively parallel signature sequencing, polony sequencing, combinatorial probe anchor synthesis, SOLiD sequencing, chain termination (e.g., Sanger sequencing), ion semiconductor sequencing, tunneling currents sequencing, heliscope single molecule sequencing, sequencing with mass spectrometry, transmission electron microscopy sequencing, RNA polymerase-based sequencing, or any other method, or a combination thereof. In some embodiments, the sequencing is a sequencing technology like Heliscope (Helicos), SMRT technology (Pacific Biosciences) or nanopore sequencing (Oxford Nanopore) that allows direct sequencing of single molecules without prior clonal amplification. In some embodiments, the sequencing is performed with or without target enrichment. In some embodiments, the sequencing is Helicos True Single Molecule Sequencing (tSMS) (e.g., as described in Harris T. D. et al., Science 320:106-109 [2008]). In some embodiments, the sequencing is 454 sequencing (Roche) (e.g., as described in Margulies, M. et al. Nature 437:376-380 (2005)). In some embodiments, the sequencing is SOLiD™ technology (Applied Biosystems). In some embodiments, the sequencing is single molecule, real-time (SMRT™) sequencing technology of Pacific Biosciences.

In some embodiments, the systems and methods described herein are used with any sequencing platform, including, but not limited to, Illumina NGS platforms, Ion Torrent (Thermo) platforms, and GeneReader (Qiagen) platforms.

In some embodiments, the sequencing is performed as described in PCT Application No. PCT/US2019/060915, entitled “Directional Targeted Sequencing,” filed Nov. 12, 2019, which is hereby incorporated by reference herein in its entirety.

In some embodiments, the sequencing reaction is a whole genome sequencing reaction (e.g., shotgun workflow). In some instances, the sequencing is digital polymerase chain reaction (PCR) sequencing. In some embodiments, the sequencing reaction is a whole transcriptome sequencing reaction (e.g., RNASeq). In some embodiments, the sequencing reaction is a panel enriched sequencing reaction. In some embodiments, the panel is pathogen-specific and/or disease condition-specific. For example, in some embodiments, the panel is a respiratory virus oligo panel (RVOP). In some embodiments, the sequencing reaction is a multiplex sequencing reaction.

In some embodiments, the method comprises determining an efficiency of one or more processing steps for the sample and/or the internal control material. For example, in some embodiments, the method comprises determining an efficiency of one or more of sample preparation, nucleic acid extraction, nucleic acid amplification, library preparation, and/or sequencing for the sample, the internal control material, and/or the one or more nucleic acid molecules originating therefrom.

In some embodiments, the method comprises comparing the efficiency of one or more processing steps between the sample and the internal control material. For example, in some instances, the efficiency of nucleic acid extraction for the one or more nucleic acid molecules originating from the first predefined category in the sample, and the efficiency of nucleic acid extraction for the one or more nucleic acid molecules originating from the internal control material, are consistent (e.g., exhibit a linear relationship). In some instances, the efficiency of nucleic acid amplification for the one or more nucleic acid molecules originating from the first predefined category in the sample, and the efficiency of nucleic acid amplification for the one or more nucleic acid molecules originating from the internal control material, are consistent (e.g., exhibit a linear relationship). In some instances, the efficiency of the sequencing reaction for the one or more nucleic acid molecules originating from the first predefined category in the sample, and the efficiency of the sequencing reaction for the one or more nucleic acid molecules originating from the internal control material, are consistent (e.g., exhibit a linear relationship). In some embodiments, the sample and internal control material efficiencies for a processing step (e.g., sample preparation, nucleic acid extraction, nucleic acid amplification, library preparation, and/or sequencing) are not consistent.

In some embodiments, the sequencing dataset comprising the first plurality of sequence reads and the second plurality of sequence reads from a sequencing of the sample including the internal control material comprises at least 1×10³, at least 1×10⁴, at least 1×10⁵, 1×10⁶, at least 1×10⁷, at least 1×10⁸, or at least 2×10⁸ sequence reads. In some embodiments, the sequencing dataset comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the sequencing dataset comprises at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, or more sequence reads. In some embodiments, the sequencing dataset consists of no more than 5×10⁷, no more than 1×10⁷, no more than 5×10⁶, no more than 4×10⁶, no more than 3×10⁶, no more than 2×10⁶, no more than 1×10⁶, no more than 500,000, no more than 100,000, no more than 50,000, no more than 30,000, no more than 20,000, no more than 10,000, no more than 9000, no more than 8000, no more than 7000, no more than 6000, no more than 5000, no more than 4000, no more than 3000, no more than 2000, no more than 1000, or less sequence reads. In some embodiments, the sequencing dataset consists of between 1000 and 5000, between 1000 and 10,000, between 2000 and 20,000, between 5000 and 50,000, between 10,000 and 100,000, between 100,000 and 500,000 between 10,000 and 500,000, between 500,000 and 1 million, between 1 million and 30 million, between 30 million and 80 million, or between 10 million and 500 million sequence reads. In some embodiments the sequencing dataset consists of a plurality of sequence reads that falls within another range starting no lower than 1000 sequence reads and ending no higher than 1×10⁹ sequence reads.

In some embodiments, the first plurality of sequence reads (e.g., originating from the first predefined category) and/or the second plurality of sequence reads (e.g., originating from the internal control material) in the sequencing dataset comprises one or more sequence reads that map (e.g., align) to a respective first reference sequence corresponding to the first predefined category (e.g., a reference genome for a microorganism) and a respective second reference sequence (e.g., a reference genome) corresponding to the internal control material.

In some embodiments, the first plurality of sequence reads (e.g., originating from the first predefined category), collectively maps to at least 50 or at least 100 base pairs of a first reference sequence (e.g., a reference genome) corresponding to the first predefined category. In some embodiments, the first plurality of sequence reads collectively maps to at least 0.1, at least 0.2, at least 0.3, at least 0.4, at least 0.5, at least 1, at least 2, at least 3, at least 4, at least 5, at least 6, at least 7, at least 8, at least 9, at least 10, or more kilobases of the first reference sequence corresponding to the first predefined category. In some embodiments, the first plurality of sequence reads collectively maps to no more than 5, no more than 4, no more than 3, no more than 2, no more than 1, no more than 0.9, no more than 0.8, no more than 0.7, no more than 0.6, no more than 0.5, no more than 0.4, no more than 0.3, no more than 0.2, no more than 0.1, or fewer kilobases of the first reference sequence corresponding to the first predefined category. In some embodiments, the first plurality of sequence reads collectively maps to between 0.1 and 0.8, between 0.3 and 1, between 0.5 and 1, between 1 and 2, between 2 and 5, between 5 and 10, or between 0.1 and 10 kilobases of the first reference sequence corresponding to the first predefined category. In some embodiments the first plurality of sequence reads collectively maps to a region of the first reference sequence that falls within another range starting no lower than 100 base pairs and ending no higher than 10,000 base pairs.

In some embodiments, the first plurality of sequence reads collectively maps to at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 30%, at least 40%, or more of the first reference sequence (e.g., reference genome) corresponding to the first predefined category. In some embodiments, the first plurality of sequence reads collectively maps to at least 50%, at least 60%, at least 70%, at least 80%, or more of the first reference sequence corresponding to the first predefined category. In some embodiments, the first plurality of sequence reads collectively maps to no more than 50%, no more than 40%, no more than 30%, no more than 20%, no more than 10%, no more than 9%, no more than 8%, no more than 7%, no more than 6%, no more than 5%, no more than 4%, no more than 3%, no more than 2%, no more than 1%, or less of the first reference sequence corresponding to the first predefined category. In some embodiments, the first plurality of sequence reads collectively maps to from 0.1% to 5%, from 0.5% to 10%, from 5% to 20%, from 20% to 50%, or from 10% to 100% of the first reference sequence corresponding to the first predefined category.

In some embodiments, the second plurality of sequence reads (e.g., originating from the internal control material) collectively maps to at least 50 or at least 100 base pairs of a second reference sequence (e.g., reference genome) corresponding to the internal control material. In some embodiments, the second plurality of sequence reads collectively maps to at least 0.1, at least 0.2, at least 0.3, at least 0.4, at least 0.5, at least 1, at least 2, at least 3, at least 4, at least 5, at least 6, at least 7, at least 8, at least 9, at least 10, or more kilobases of the second reference sequence corresponding to the internal control material. In some embodiments, the second plurality of sequence reads collectively maps to no more than 5, no more than 4, no more than 3, no more than 2, no more than 1, no more than 0.9, no more than 0.8, no more than 0.7, no more than 0.6, no more than 0.5, no more than 0.4, no more than 0.3, no more than 0.2, no more than 0.1, or fewer kilobases of the second reference sequence corresponding to the internal control material. In some embodiments, the second plurality of sequence reads collectively maps to between 0.1 and 0.8, between 0.3 and 1, between 0.5 and 1, between 1 and 2, between 2 and 5, between 5 and 10, or between 0.1 and 10 kilobases of the second reference sequence corresponding to the internal control material. In some embodiments the second plurality of sequence reads collectively maps to a region of the second reference sequence that falls within another range starting no lower than 100 base pairs and ending no higher than 10,000 base pairs.

In some embodiments, the second plurality of sequence reads collectively maps to at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 30%, at least 40%, or more of the second reference sequence (e.g., reference genome) corresponding to the internal control material. In some embodiments, the second plurality of sequence reads collectively maps to at least 50%, at least 60%, at least 70%, at least 80%, or more of the second reference sequence corresponding to the internal control material. In some embodiments, the second plurality of sequence reads collectively maps to no more than 50%, no more than 40%, no more than 30%, no more than 20%, no more than 10%, no more than 9%, no more than 8%, no more than 7%, no more than 6%, no more than 5%, no more than 4%, no more than 3%, no more than 2%, no more than 1%, or less of the second reference sequence corresponding to the internal control material. In some embodiments, the second plurality of sequence reads collectively maps to from 0.1% to 5%, from 0.5% to 10%, from 5% to 20%, from 20% to 50%, or from 10% to 100% of the second reference sequence corresponding to the internal control material.

In some embodiments, the sequencing dataset further includes a third plurality of sequence reads, where each respective sequence read in the third plurality of sequence reads is determined by a sequencing of a nucleic acid molecule in the one or more nucleic acid molecules originating from the source other than the first predefined category. In some embodiments, the third plurality of sequence reads comprises sequence reads originating from a host organism (e.g., where the first predefined category is a microorganism). In some embodiments, the third plurality of sequence reads comprises sequence reads originating from a human (e.g., a patient).

In some embodiments, the third plurality of sequence reads comprises one or more sequence reads that map (e.g., align) to a respective third reference sequence corresponding to the source other than the first predefined category. For example, in some embodiments, the third plurality of sequence reads comprises one or more sequence reads that map to a human reference genome.

In some embodiments, the sequencing dataset further includes a fourth plurality of sequence reads, where each respective sequence read in the fourth plurality of sequence reads is determined by a sequencing of a nucleic acid molecule in the one or more nucleic acid molecules originating from a second predefined category other than the first predefined category. In some embodiments, the fourth plurality of sequence reads comprises sequence reads originating from a co-infecting and/or co-contaminating microorganism (e.g., where the first predefined category is an infecting and/or contaminating microorganism). In some embodiments, the fourth plurality of sequence reads comprises sequence reads originating from a pathogen.

In some embodiments, the fourth plurality of sequence reads comprises one or more sequence reads that map (e.g., align) to a respective fourth reference sequence corresponding to the second predefined category other than the first predefined category. For example, in some embodiments, the fourth plurality of sequence reads comprises one or more sequence reads that map to a reference genome corresponding to a second microorganism other than the first microorganism.

In some embodiments, the third, fourth, and/or any subsequent pluralities of sequence reads include any of the embodiments disclosed herein as for the first and/or second pluralities of sequence reads, as well as any substitutions, modifications, additions, deletions, and/or combinations thereof, as will be apparent to one skilled in the art.

Obtaining Normalized Read Counts.

Referring to Block 208, the method disclosed herein further comprises determining, from the first plurality of sequence reads, a first normalized read count for the number of sequence reads originating from the first predefined category, where the first normalized read count is normalized based on a first target nucleotide sequence length.

Additionally, referring to Block 210, the method further comprises determining, from the second plurality of sequence reads, a second normalized read count for the number of sequence reads originating from the internal control material, where the second normalized read count is normalized based on a second target nucleotide sequence length.

In some embodiments, the determining the first read count and the second read count further comprises mapping (e.g., aligning) the first plurality of sequence reads to all or a portion of a first reference sequence corresponding to the first predefined category (e.g., a first reference genome for a microorganism), and mapping (e.g., aligning) the second plurality of sequence reads to all or a portion of a second reference sequence corresponding to the internal control material (e.g., a reference genome, a naturally occurring nucleotide sequence, and/or a synthetic nucleotide sequence).

In some embodiments, the mapping comprises aligning and/or assembling one or more sequence reads in one or more of the first and the second plurality of sequence reads. In some embodiments, the alignment and/or assembly comprises one or more alignment algorithms that detect overlapping and/or redundant sequence information in each respective plurality of sequence reads. In some embodiments, the alignment and/or assembly is based at least in part on a known reference sequence (e.g., an alignment using a variant of the center-star algorithm). In some implementations, the alignment and/or assembly comprises one or more alignment algorithms that align sequence reads relative to each other without using a reference sequence (e.g., de novo assembly routines). Non-limiting examples of alignment methods include BLASR (basic local alignment with successive refinement), PHRAP, CAP, ClustalW, T-Coffee, AMOS make-consensus, and/or other dynamic programming multiple sequence alignments (MSAs). In some embodiments, the mapping is performed using a k-mer alignment (e.g., with and/or without a reference sequence).

In some embodiments, the analysis comprises pre-processing and/or pre-sorting of one or more sequence reads in the sequencing dataset. In some embodiments, pre-sorting includes sorting each sequence read obtained from the sequencing of the sample including the internal control material into one or more bins, where each bin corresponds to a different nucleic acid source (e.g., the first predefined category, the source other than the first predefined category, and/or the internal control material), depending on the likelihood that the sequence read originated from the respective source. Each sequence read is then mapped (e.g., using a k-mer alignment, a gapped k-mer alignment, and/or a full alignment) to one or more reference sequences (e.g., genomes) corresponding to different sources. In some embodiments, the analysis is performed using an analysis pipeline. Methods of mapping sequence reads obtained from sequencing nucleic acids are further provided in, for example, U.S. patent application Ser. No. 15/724,476, entitled “Methods and Systems for Multiple Taxonomic Classification,” filed Oct. 4, 2017, and U.S. Patent Application No. 62/723,384, entitled “Methods and Systems for Providing Sample Information,” filed Aug. 27, 2018, each of which is hereby incorporated by reference in its entirety.

In some embodiments, the mapping is performed using a mapping (e.g., alignment) tool, including, but not limited to, BLAST, BLASR, BWA-MEM, DAMAPPER, NGMLR, GraphMap, Minimap, and/or Velvet. In some embodiments, the mapping tool performs the mapping using a reference sequence (e.g., a reference genome). In some embodiments, the mapping tool performs the mapping without the use of a reference sequence. For example, BGREAT (see, Limasset et al., 2016, BMC Bioinformatics 17:237) and deBGA (e.g., as described by Liu et al., 2016, Bioinformatics 32(21):3224-3232) are designed to work with both second generation sequencing data and de Bruijn graphs as opposed to linear target sequences. Other methods include BlastGraph to use BLAST mapping results to cluster alignments and perform comparative genomic analyses (as described in Ye et al., 2013, Bioinformatics 29(24):3222-3224), and/or GramTools to map short reads to a population reference graph (e.g., as described in Maciuca et al., 2016, on the Internet at dx.doi.org/10.1101/059170). See also, Zerbino and Birney, “Velvet: Algorithms for de novo short read assembly using de Bruijn graphs,” Genome Reach 2008, 18:821-829. In some embodiments, the mapping is performed by mapping nucleotide sequences (e.g., obtained from a sequencing of nucleic acid molecules) to a nucleotide reference sequence (e.g., a genomic and/or transcriptomic reference sequence). In some embodiments, the mapping is performed by mapping polypeptide sequences (e.g., obtained from a translation of one or more nucleotide sequences obtained from a sequencing of nucleic acid molecules) to a polypeptide reference sequence (e.g., an amino acid sequence for a protein product). In some embodiments, a nucleotide and/or polypeptide reference sequence corresponds to a microorganism. In some embodiments, the nucleotide and/or polypeptide reference sequence is obtained from a database (e.g., a microorganism database as disclosed herein).

Other methods of mapping sequence reads to a reference sequence are possible, as will be apparent to one skilled in the art. See, for example, Roumpeka et al., 2017, “A Review of Bioinformatics Tools for Bio-Prospecting from Metagenomic Sequence Data,” Front. Genet. 8:23, doi: 10.3389/fgene.2017.00023, which is hereby incorporated herein by reference in its entirety. In some embodiments, the sequencing, mapping, and/or analysis is performed using a software program (e.g., Explify), as described in Example 1 (Examples, below). See, for example, IDbyDNA, 2019, “Explify Software v1.5.0 User Manual,” Document No. TH-2019-200-006, pp. 1-44, which is hereby incorporated by reference herein in its entirety.

In some embodiments, a reference sequence is a reference genome for a microorganism. In some implementations, reference sequences and reference genomes are any of the embodiments disclosed herein (see, for example, Definitions: “Reference genomes” and Definitions: “Reference sequences”, above).

In some embodiments, the read count is a read depth (see, for example, Definitions: Depth). For example, in some embodiments, the read count is a read depth obtained from an alignment of a plurality of sequence reads. In some embodiments, the read count is a read depth obtained for a plurality of sequence reads that map to a target nucleotide sequence (e.g., a target region in a reference sequence). In some embodiments, the read count is the total count of sequence reads that map, all or in part (e.g., partial and/or overlapping) to all or a portion of the target nucleotide sequence. In some embodiments, the read count is a measure of the depth at each nucleotide base in the target nucleotide sequence. For example, in some such embodiments, the read count is the mean sequencing depth at each nucleotide base in the target nucleotide sequence, averaged over the length of the target nucleotide sequence.

In some embodiments, the read count (e.g., depth) is at least 0.1×, at least 0.2×, at least 0.3×, at least 0.4×, at least 0.5×, at least 0.6×, at least 0.7×, at least 0.8×, at least 0.9×, at least 1×, at least 2×, at least 3×, at least 4×, at least 5×, at least 6×, at least 7×, at least 8×, at least 9×, at least 10×, or more. In some embodiments, the read count (e.g., depth) is at least 10×, at least 20×, at least 30×, at least 40×, at least 50×, at least 60×, at least 70×, at least 80×, at least 90×, at least 100×, at least 200×, at least 300×, at least 400×, at least 500×, at least 600×, at least 700×, at least 800×, at least 900×, at least 1000×, at least 2000×, at least 5000×, at least 10,000×, at least 20,000×, at least 30,000×, or more. In some embodiments, the read count (e.g., depth) is no more than 1000×, no more than 500×, no more than 100×, no more than 90×, no more than 80×, no more than 70×, no more than 60×, no more than 50×, no more than 40×, no more than 30×, no more than 20×, no more than 10×, no more than 5×, or less. In some embodiments, for instance in shotgun sequencing, the read count (e.g., depth) is at least 0.001×, or at least 0.01×. In some embodiments, the read count (e.g., depth) is between 0.0005× and 0.10×.

In some implementations, the determining the first read count and the second read count further comprises normalizing read counts against a target nucleotide sequence length. For example, in some embodiments, the obtaining normalized read counts comprises determining a first count of the number of sequence reads, in the first plurality of sequence reads, that map to a first target nucleotide sequence obtained from the first reference sequence corresponding to the first predefined category, determining a second count of the number of sequence reads, in the second plurality of sequence reads, that map to a second target nucleotide sequence obtained from the second reference sequence corresponding to the internal control material, normalizing the first count based on the length of the first target nucleotide sequence, and normalizing the second count based on the length of the second target nucleotide sequence, thus obtaining the first normalized read count and the second normalized read count, respectively.

In some embodiments, normalization is performed by normalizing a read count by, for example, the total number of reads, the total number of reads associated with a target nucleotide sequence, the length of the reference sequence, and/or a combination thereof. Examples of such normalization include fragments per kilobase of transcript per million mapped reads (FPKM) and/or reads per kilobase of transcript per million mapped reads (RPKM). In some embodiments, normalization includes other methods that take into account the relative amount of reads in different samples, such as normalizing sequencing reads from samples by the median of ratios of observed counts per sequence. Thus, in some embodiments, the first normalized read count and the second normalized read count are expressed as reads per kilobase per million mapped reads (RPKM). RPKM can be calculated using the equation:

RPKM=(targetcount*10³*10⁶)/(totalcount*targetlength), where targetcount indicates the number of sequence reads that map to the target nucleotide sequence, totalcount indicates the total number of sequence reads obtained from the sequencing of the sample, and targetlength indicates the length of the target nucleotide sequence in base pairs.

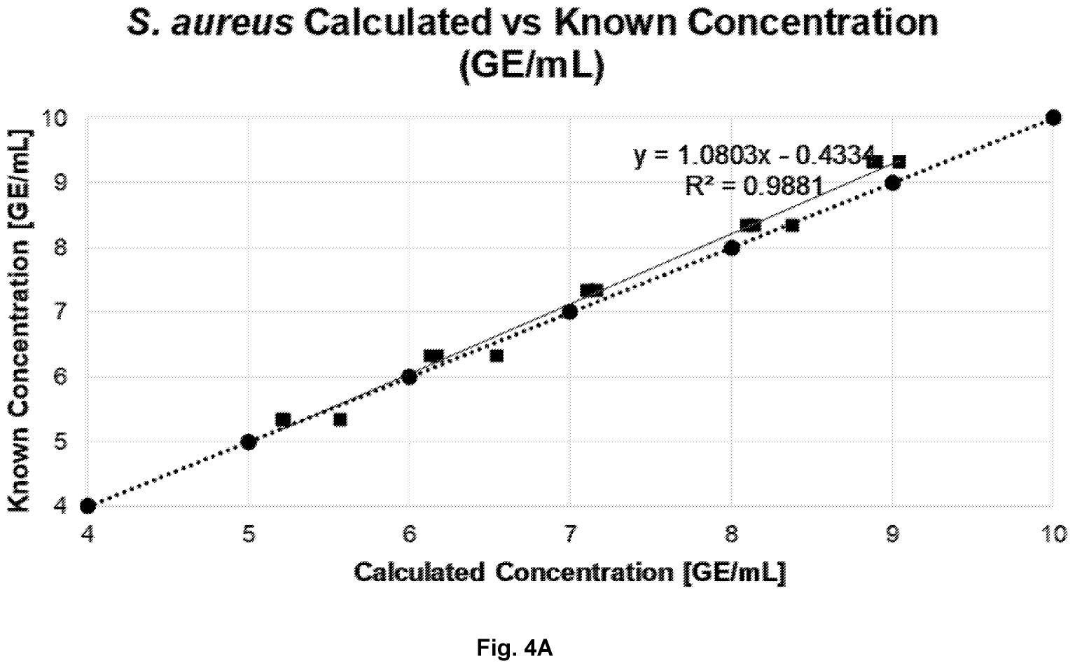

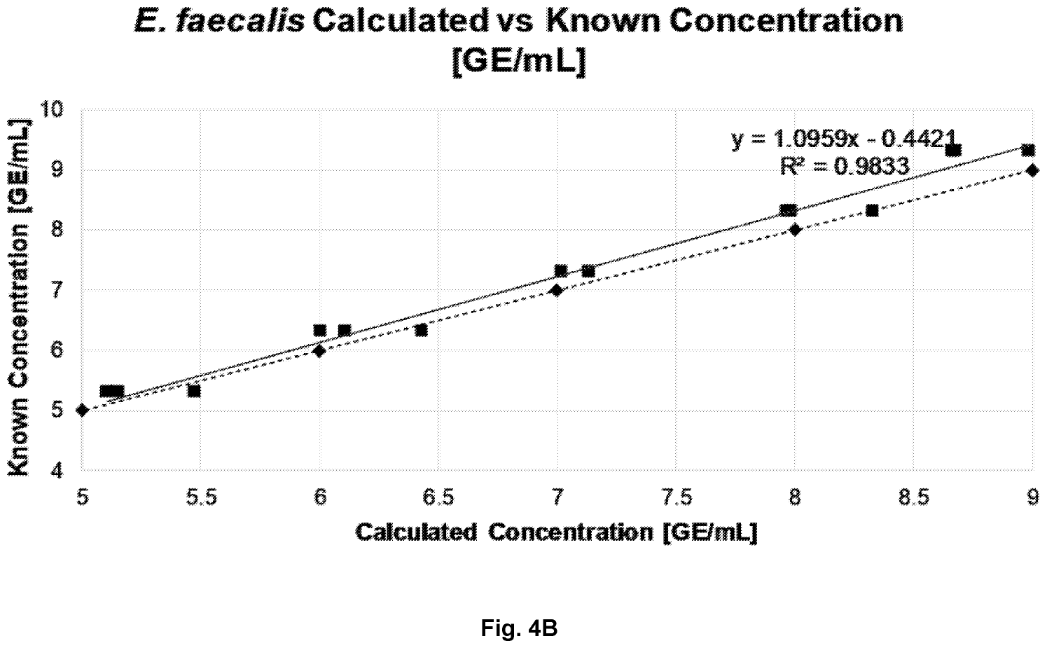

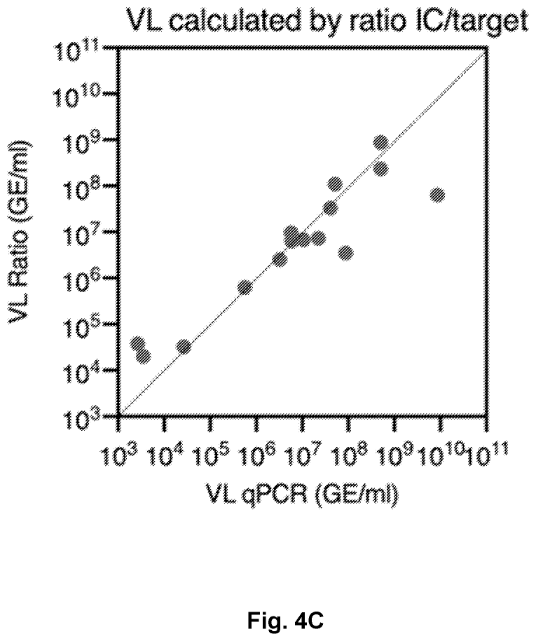

In some embodiments, normalization of read counts is performed by obtaining an aggregated RPKM across a plurality of target nucleotide subsequences. For example, as illustrated in Example 3 and FIGS. 4A and 4B below, normalized read counts for Staphylococcus aureus, Enterococcus faecalis, and the IC material in MCS titration samples were calculated as the aggregate RPKM, where the target length and number of reads mapped were aggregated across the entire targeted region, including contiguous and non-contiguous bases, using the formula for RPKM provided above.

In some embodiments, an Alternative Normalized Read Count calculation is used. For example, in some instances, alternative normalized read counts can provide more robust results in clinical practice where it can reasonably be expected that circulating strains are gaining and losing genetic material and may not contain every targeted region. One such calculation is a median RPKM, where the RPKM of each non-contiguous target region is calculated, and then the median non-contiguous target region RPKM is used to represent the predefined category's normalized read count.

In some embodiments, the normalized read count is obtained by incorporating targeted region outlier removal upstream of the aggregate RPKM or median RPKM calculation. For example, in some instances, targeted regions yielding low read support evidence are excluded from the predefined category's normalized read count calculation.

In some embodiments, the target nucleotide sequence is determined for each source of sequence reads (e.g., for a first predefined category, a source other than the first predefined category, and/or the internal control material). Thus, in some embodiments, the first target nucleotide sequence length and the second target nucleotide sequence length are different.

In some embodiments, the first target nucleotide sequence length is determined from all or a portion of a reference sequence (e.g., a reference genome) corresponding to the first predefined category. In some embodiments, the first target nucleotide sequence length is determined from at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of a reference sequence corresponding to the first predefined category. In some embodiments, the first target nucleotide sequence length comprises at least two at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of a reference sequence corresponding to the first predefined category. In some embodiments, the first target nucleotide sequence length is determined from a single contiguous region of a reference sequence corresponding to the first predefined category.

In some embodiments, the first target nucleotide sequence length comprises at least 50 or at least 100 base pairs (e.g., contiguous and/or non-contiguous base pairs). In some embodiments, the first target nucleotide sequence length comprises at least 10, at least 100, at least 500, at least 1000, at least 2000, at least 3000, at least 4000, at least 5000, at least 6000, at least 7000, at least 8000, at least 9000, at least 10,000, at least 20,000 base pairs (e.g., contiguous and/or non-contiguous base pairs), or more. In some embodiments, the first target nucleotide sequence length comprises no more than 10,000, no more than 9000, no more than 8000, no more than 7000, no more than 6000, no more than 5000, no more than 4000, no more than 3000, no more than 2000 base pairs (e.g., contiguous and/or non-contiguous base pairs), or less. In some embodiments, the first target nucleotide sequence length consists of from 10 to 500, from 100 to 1000, from 300 to 5000, from 1000 to 8000, from 5000 to 20,000, or from 100 to 20,000 base pairs (e.g., contiguous and/or non-contiguous base pairs). In some embodiments the first target nucleotide sequence length consists of another range starting no lower than 100 base pairs and ending no higher than 20,000 base pairs.

In some embodiments, the first target nucleotide sequence length comprises at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 30%, at least 40%, or more of the first reference sequence (e.g., reference genome) corresponding to the first predefined category (e.g., contiguous and/or non-contiguous regions of the reference sequence). In some embodiments, the first target nucleotide sequence length comprises at least 50%, at least 60%, at least 70%, at least 80%, or more of the first reference sequence corresponding to the first predefined category (e.g., contiguous and/or non-contiguous regions of the reference sequence). In some embodiments, the first target nucleotide sequence length consists of no more than 50%, no more than 40%, no more than 30%, no more than 20%, no more than 10%, no more than 9%, no more than 8%, no more than 7%, no more than 6%, no more than 5%, no more than 4%, no more than 3%, no more than 2%, no more than 1%, or less of the first reference sequence corresponding to the first predefined category (e.g., contiguous and/or non-contiguous regions of the reference sequence). In some embodiments, the first target nucleotide sequence length consists of from 0.1% to 5%, from 0.5% to 10%, from 5% to 20%, from 20% to 50%, or from 10% to 100% of the first reference sequence corresponding to the first predefined category (e.g., contiguous and/or non-contiguous regions of the reference sequence). In some embodiments, the first target nucleotide sequence length comprises at least 0.001% or at least 0.01% of the first reference sequence corresponding to the first predefined category (e.g., contiguous and/or non-contiguous regions of the reference sequence). In some embodiments, the first target nucleotide sequence length consists of between 0.001% and 1% of the first reference sequence corresponding to the first predefined category (e.g., contiguous and/or non-contiguous regions of the reference sequence). In some embodiments, the first target nucleotide sequence length consists of between 0.001% and 3% of the first reference sequence corresponding to the first predefined category (e.g., contiguous and/or non-contiguous regions of the reference sequence).

In some embodiments, the first target nucleotide sequence length is a fixed length. In some embodiments, the first target nucleotide sequence length is a constant value that is determined based on the reference sequence corresponding to the respective first predefined category.

In some embodiments, the second target nucleotide sequence length is determined from all or a portion of a reference sequence (e.g., a reference genome, a natural sequence, and/or a synthetic sequence) corresponding to the internal control material. In some embodiments, the second target nucleotide sequence length is determined from at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of a reference sequence corresponding to the internal control material. In some embodiments, the second target nucleotide sequence length comprises at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of a reference sequence corresponding to the internal control material. In some embodiments, the second target nucleotide sequence length is determined from a single contiguous region of a reference sequence corresponding to the internal control material.

In some embodiments, the second target nucleotide sequence length comprises at least 50 base pairs or at least 100 base pairs (e.g., contiguous and/or non-contiguous base pairs). In some embodiments, the second target nucleotide sequence length comprises at least 10, at least 100, at least 500, at least 1000, at least 2000, at least 3000, at least 4000, at least 5000, at least 6000, at least 7000, at least 8000, at least 9000, at least 10,000, at least 20,000 base pairs (e.g., contiguous and/or non-contiguous base pairs), or more. In some embodiments, the second target nucleotide sequence length consists of no more than 10,000, no more than 9000, no more than 8000, no more than 7000, no more than 6000, no more than 5000, no more than 4000, no more than 3000, no more than 2000 base pairs (e.g., contiguous and/or non-contiguous base pairs), or less. In some embodiments, the second target nucleotide sequence length consists of from 10 to 500, from 100 to 1000, from 300 to 5000, from 1000 to 8000, from 5000 to 20,000, or from 100 to 20,000 base pairs (e.g., contiguous and/or non-contiguous base pairs). In some embodiments the second target nucleotide sequence length comprises another range starting no lower than 100 base pairs and ending no higher than 20,000 base pairs.

In some embodiments, the second target nucleotide sequence length comprises at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 30%, at least 40%, or more of the second reference sequence (e.g., reference genome) corresponding to the internal control material (e.g., contiguous and/or non-contiguous regions of the reference sequence). In some embodiments, the second target nucleotide sequence length comprises at least 50%, at least 60%, at least 70%, at least 80%, or more of the second reference sequence corresponding to the internal control material (e.g., contiguous and/or non-contiguous regions of the reference sequence). In some embodiments, the second target nucleotide sequence length consists of no more than 50%, no more than 40%, no more than 30%, no more than 20%, no more than 10%, no more than 9%, no more than 8%, no more than 7%, no more than 6%, no more than 5%, no more than 4%, no more than 3%, no more than 2%, no more than 1%, or less of the second reference sequence corresponding to the internal control material (e.g., contiguous and/or non-contiguous regions of the reference sequence). In some embodiments, the second target nucleotide sequence length consists of from 0.1% to 5%, from 0.5% to 10%, from 5% to 20%, from 20% to 50%, or from 10% to 100% of the second reference sequence corresponding to the internal control material (e.g., contiguous and/or non-contiguous regions of the reference sequence).

In some embodiments, the second target nucleotide sequence length is a fixed length. In some embodiments, the second target nucleotide sequence length is a constant value that is determined based on the reference sequence corresponding to the respective internal control material.

In some implementations, the analysis further comprises detecting and/or identifying the presence, absence, and/or identity of the predefined category (e.g., microorganism) in the sample. In some implementations, the analysis further comprises detecting and/or identifying the presence, absence, and/or identity of an antimicrobial resistance gene in the predefined category (e.g., microorganism) in the sample. In some embodiments, an antimicrobial resistance gene is any of the embodiments disclosed herein (see, for example, Definitions: “Antimicrobial resistance,” above).

Quantifying Predefined Categories.

Referring to Block 212, the method disclosed herein further comprises calculating the amount of the first predefined category in the sample based on the first normalized read count, the second normalized read count, and the known quantity of the internal control material.

For example, referring to Block 214, in some embodiments, the calculating the amount of the first predefined category in the sample is determined based on the relationship Q_{org}=(Q_{IC}*RC_{org})/RC_{IC}, where Q_{org} is the amount (e.g., concentration) of the first predefined category in the sample, Q_{IC} is the known quantity (e.g., concentration) of the internal control material, RC_{org} is the first normalized read count for the number of sequence reads originating from the first predefined category, and RC_{IC} is the second normalized read count for the number of sequence reads originating from the internal control material.

In some embodiments, the known quantity of the internal control material and/or the calculated amount of the predefined category is expressed in any suitable unit for quantification, including genomic or transcriptomic concentration by volume or weight (e.g., copies/mL, GE/mL, IU/mL, copies/weight, etc.).

In some embodiments, the first read count is any observed read count for the number of sequence reads originating from the first predefined category. In some embodiments, the first read count is a variable determined based on variations in one or more of sample type, sample aliquot, sample processing, nucleic acid extraction, nucleic acid amplification, sequencing reaction, sequencing run, and/or other workflow protocols.

In some embodiments, the second read count is any observed read count for the number of sequence reads originating from the internal control material. In some embodiments, the second read count is a variable determined based on variations in one or more of sample type, sample aliquot, sample processing, nucleic acid extraction, nucleic acid amplification, sequencing reaction, sequencing run, and/or other workflow protocols.

In some embodiments, the method comprises determining an amount of the predefined category independent of a limit of detection filter for the first and/or second read count. In some embodiments, the method comprises determining an amount of the predefined category independent of a minimum and/or maximum read count threshold for the first and/or second read count.

In some embodiments, to account for variability in sampling and measurement that can present in one or more of the foregoing workflow processes, the method comprises applying one or more correction factors to the calculation of the amount of the predefined category in the sample. For example, in some embodiments, assay-specific (e.g., predefined category-specific and/or target-specific) correction factors are used to correct for repeatable and systematic factors like differences in nucleic acid amplification efficiency, differences in nucleic acid purification efficiency, differences in sequencing library preparation, and/or differences in sequencing efficiency. Since such differences are repeatable and systematic for a given sample, analyte, and/or assay, in some embodiments, the differences can be measured and used to generate assay-specific correction factors to correct predefined category quantification. In some embodiments, a plurality of assay-specific (e.g., predefined category-specific and/or target-specific) correction factors are applied to a plurality of predefined categories for quantification to remove systematic differences in target quantification performance for each predefined category in the plurality of predefined categories.

In some embodiments, the amount of the first predefined category in the sample determined by the relationship Q_{org}=(Q_{IC}*RC_{org})/RC_{IC} is corrected by one or more correction factors. In some embodiments, the one or more correction factors comprises an extraction correction factor. In some embodiments, the one or more correction factors comprises a sequencing correction factor. In some embodiments, the one or more correction factors comprises an abundance correction factor. In some embodiments, the one or more correction factors comprises any one or more of an extraction correction factor, a sequencing correction factor, and/or an abundance correction factor, and/or any combination thereof.

Accordingly, in some embodiments, the method comprises correcting the amount of the first predefined category in the sample using an extraction correction factor (e.g., a predefined category-specific correction factor (EF) to account for differences in extraction efficiency). In some embodiments, the extraction correction factor is obtained based on a sequencing of a known amount of one or more extraction correction sequences in a plurality of extraction correction sequences. In some embodiments, the plurality of extraction correction sequences comprises sequences from a representative set of predefined categories (e.g., for correcting predefined category-specific differences in extraction efficiency).

In some embodiments, an extraction correction sequence in the plurality of extraction correction sequences comprises all or a portion of a reference sequence (e.g., a reference genome) corresponding to a predefined category in a plurality of predefined categories. In some embodiments, each extraction correction sequence in the plurality of extraction correction sequences comprises all or a portion of a reference sequence (e.g., a reference genome) corresponding to a predefined category in a plurality of predefined categories. In some embodiments, the plurality of extraction correction sequences comprises all or a portion of a first reference sequence corresponding to the first predefined category (e.g., a reference genome for a target microorganism for quantification). In some embodiments, the extraction correction factor is averaged over a plurality of extraction correction sequences (e.g., grouped by species, strain, and/or other taxonomic classification). Example strategies for determining extraction correction factors are provided in Table 2.

TABLE 2
Example Strategies for Extraction Correction Factors

| | EF (no | EF | EF (group |
|---|---|---|---|
| | correction) | (explicit) | average) |
| Organism 1 (Gram+) | 1 | 0.9 | 0.85 |
| Organism 2 (Gram+) | 1 | 0.8 | 0.85 |
| Organism 3 (Gram+) | 1 | 0.7 | 0.65 |
| Organism 4 (Gram+) | 1 | 0.6 | 0.65 |
| Organism 5 (Gram+) | 1 | 1.1 | 1.05 |
| Organism 6 (Gram+) | 1 | 1.0 | 1.05 |

In some embodiments, the extraction correction factor is a fixed value.

In some embodiments, the method comprises correcting the amount of the first predefined category in the sample using a sequencing correction factor (e.g., a target-specific correction factor (SF) to account for differences in sequencing efficiency). In some embodiments, the sequencing correction factor is obtained based on a sequencing of a known amount of one or more sequencing-correction sequences in a plurality of sequencing-correction sequences. In some embodiments, the plurality of sequencing-correction sequences comprises sequences for a representative set of target regions in a reference sequence (e.g., for correcting target-specific differences in sequencing efficiency).

In some embodiments, a sequencing-correction sequence in the plurality of sequencing-correction sequences comprises all or a portion of a reference sequence (e.g., a reference genome) corresponding to a predefined category in a plurality of predefined categories. In some embodiments, each sequencing-correction sequence in the plurality of sequencing-correction sequences comprises all or a portion of a reference sequence (e.g., a reference genome) corresponding to a predefined category in a plurality of predefined categories. In some embodiments, the plurality of sequencing-correction sequences comprises all or a portion of a first target nucleotide sequence corresponding to the first predefined category. In some embodiments, the sequencing correction factor is averaged over a plurality of sequencing-correction sequences (e.g., grouped by species, strain, and/or other taxonomic classification). Example strategies for determining sequencing correction factors are provided in Table 3.

TABLE 3
Example Strategies for Sequencing Correction Factors

| | SF (no correction) | SF (explicit) | SF (group average) |
|---|---|---|---|
| Sequence 1 | 1 | 0.9 | 0.85 |
| Sequence 2 | 1 | 0.8 | 0.85 |
| Sequence 3 | 1 | 0.7 | 0.65 |
| Sequence 4 | 1 | 0.6 | 0.65 |
| Sequence 5 | 1 | 1.1 | 1.05 |
| Sequence 6 | 1 | 1.0 | 1.05 |

In some embodiments, the sequencing correction factor is a fixed value.

In some embodiments, the method comprises correcting the amount of the first predefined category in the sample using an abundance correction factor (e.g., to account for biological differences in abundances of target sequences, such as copy number variations).

In some embodiments, the abundance correction factor is obtained based on a sequencing of a known amount of one or more abundance correction sequences in a plurality of abundance correction sequences. In some embodiments, the plurality of abundance correction sequences comprises sequences from a representative set of predefined categories and/or target sequences (e.g., regions comprising copy number variations). In some embodiments, an abundance correction sequence in the plurality of abundance correction sequences comprises all or a portion of a reference sequence (e.g., a reference genome) corresponding to one or more predefined categories in a plurality of predefined categories (e.g., populations and/or predefined categories comprising genomic copy number variations). In some embodiments, each abundance correction sequence in the plurality of abundance correction sequences comprises all or a portion of a reference sequence (e.g., a reference genome) corresponding to a predefined category in a plurality of predefined categories (e.g., populations and/or predefined categories comprising genomic copy number variations). In some embodiments, the plurality of abundance correction sequences comprises all or a portion of a first reference sequence corresponding to the first predefined category (e.g., a reference genome, comprising a copy number variation, for a target microorganism for quantification). In some embodiments, the abundance correction factor is averaged over a plurality of abundance correction sequences (e.g., grouped by species, strain, and/or other taxonomic classification). In some embodiments, the abundance correction factor is a fixed value.

In some embodiments, one or more correction factors are applied to the quantification methods disclosed herein by scaling (e.g., multiplying) the amount of the first predefined category in the sample Q_{org} by the respective one or more correction factors (e.g., an extraction correction factor, a sequencing correction factor, and/or an abundance correction factor). For example, in some such embodiments, the amount of the first predefined category in the sample is corrected based on the relationship Q_{org}=(AF*EF*SF*Q_{IC}*RC_{org})/RC_{IC}, where AF is an abundance correction factor, EF is an extraction correction factor, SF is a sequencing correction factor, Q_{org} is the amount of the first predefined category in the sample, Q_{IC} is the known quantity of the internal control material, RC_{org} is the first normalized read count for the number of sequence reads originating from the first predefined category, and RC_{IC} is the second normalized read count for the number of sequence reads originating from the internal control material.

Quantification of Multiple Populations.

In some embodiments, the sequencing dataset further includes a third plurality of sequence reads, wherein each respective sequence read in the third plurality of sequence reads is determined by a sequencing of a nucleic acid molecule in the one or more nucleic acid molecules originating from the source other than the first predefined category. In some embodiments, the source other than the first predefined category is human.

In some embodiments, the method further comprises mapping (e.g., aligning) the third plurality of sequence reads to all or a portion of a third reference sequence corresponding to the source other than the first predefined category (e.g., a human reference genome); determining a third count of the number of sequence reads, in the third plurality of sequence reads, that map to a third target nucleotide sequence obtained from the third reference sequence corresponding to the source other than the first predefined category; normalizing the third count based on the length of the third target nucleotide sequence, thereby determining a third normalized read count for the number of sequence reads originating from the source other than the first predefined category; and calculating the amount of the first predefined category in the sample based at least in part on the third normalized read count.

In some embodiments, the third normalized read count is expressed as reads per kilobase per million mapped reads (RPKM).

In some embodiments, the third target nucleotide sequence length is determined from at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of the third reference sequence corresponding to the source other than the first predefined category (e.g., a human reference genome). In some embodiments, the third target nucleotide sequence length comprises at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of the third reference sequence corresponding to the source other than the first predefined category (e.g., a human reference genome). In some embodiments, the third target nucleotide sequence length consists of between (i) 2, 3, 4, 5, 6, 7, 8, 9, 10, 15, 20, 25, 30, 35, 40, or 45 and (ii) 50, 100, 200, 500, or 1,000 non-contiguous regions of the third reference sequence corresponding to the source other than the first predefined category (e.g., a human reference genome). In some embodiments, the third target nucleotide sequence length is determined from a single contiguous region of the third reference sequence corresponding to the source other than the first predefined category. In some embodiments, the third plurality of sequence reads collectively maps to at least 50 base pairs or at least 100 base pairs of a third reference sequence corresponding to the source other than the first predefined category.

Other embodiments for the third plurality of sequence reads, the third reference sequence, the third target nucleotide sequence, sequencing, mapping sequence reads, obtaining read counts, normalization, quantification, and any characteristics or elements thereof, are possible. For example, any of the embodiments described herein for a plurality of sequence reads, a reference sequence, and a target nucleotide sequence, sequencing, mapping sequence reads, obtaining read counts, normalization, quantification, and any other characteristics or elements thereof, are applicable to the third instance as to the first and/or the second instance. Further, any substitutions, modifications, additions, deletions, and/or combinations of any of the systems and methods provided herein are possible, as will be apparent to one skilled in the art.

Another aspect of the present disclosure provides a method for determining an amount of a plurality of predefined categories in the sample, where the sample comprises, for each respective predefined category in the plurality of predefined categories, one or more nucleic acid molecules originating from the respective predefined category (e.g., a plurality of co-infecting and/or co-contaminating population of microorganisms). For example, as described above, in some embodiments, the plurality of predefined categories comprises at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, or more predefined categories (e.g., populations of microorganisms in the sample). In some embodiments, the method is used to determine an amount of at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100, at least 200, at least 300, at least 400, at least 500, at least 600, at least 700, at least 800, at least 900, at least 1000, at least 2000, or more predefined categories (e.g., populations of microorganisms in the sample).

In some embodiments, the plurality of predefined categories comprises no more than 5,000, no more than 3000, no more than 2000, no more than 1000, no more than 500, no more than 300, no more than 200, no more than 100, no more than 50, no more than 40, no more than 30, no more than 20, no more than 10, or fewer predefined categories. In some embodiments, the method is used to determine an amount of no more than 5,000, no more than 3000, no more than 2000, no more than 1000, no more than 500, no more than 300, no more than 200, no more than 100, no more than 50, no more than 40, no more than 30, no more than 20, no more than 10, or fewer predefined categories. In some embodiments, the plurality of predefined categories consists of from 1 to 10, from 5 to 20, from 10 to 50, from 50 to 100, from 80 to 1000, or from 500 to 2000 predefined categories. In some embodiments, the method is used to determine an amount of from 1 to 10, from 5 to 20, from 10 to 50, from 50 to 100, from 80 to 1000, or from 500 to 2000 predefined categories. In some embodiments, the plurality of predefined categories comprises another range starting no lower than 2 sequence reads and ending no higher than 3000 predefined categories.

Accordingly, in some embodiments, the first predefined category is in a plurality of predefined categories in the sample, and the dataset comprises a corresponding plurality of sequence reads for each predefined category in the plurality of predefined categories, including the first plurality of sequence reads for the first predefined category. In some such embodiments, the method further comprises, for each respective predefined category beyond the first predefined category in the plurality of predefined categories, determining a respective normalized read count for the number of sequence reads originating from the respective predefined category, where the respective normalized read count is normalized based on a corresponding target nucleotide sequence length for the respective predefined category, and calculating the amount of the respective predefined category in the sample based on the respective normalized read count for the number of sequence reads originating from the respective predefined category, the second normalized read count, and the known quantity of the internal control material.

In some embodiments, a respective predefined category beyond the first predefined category in the plurality of predefined categories is a microorganism. In some embodiments, each respective predefined category beyond the first predefined category in the plurality of predefined categories is a microorganism. In some embodiments, the microorganism is selected from the group consisting of bacterial, fungal, viral, and parasitic. In some embodiments, the microorganism is a pathogen.

In some embodiments, the amount of the first predefined category in the sample and the amount of a respective predefined category, other than the first predefined category, in the plurality of predefined categories in the sample are different.

In some embodiments, the sequencing dataset further includes a respective plurality of sequence reads, for each respective predefined category other than the first predefined category in the plurality of predefined categories, where each respective sequence read in the respective plurality of sequence reads is determined by a sequencing of a nucleic acid molecule in the one or more nucleic acid molecules originating from the respective predefined category. In some embodiments, the respective plurality of sequence reads collectively maps to at least 50 base pairs or at least 100 base pairs of a reference sequence (e.g., a reference genome) corresponding to the respective predefined category.

In some embodiments, the method further comprises mapping (e.g., aligning), for each respective predefined category beyond the first predefined category in the plurality of predefined categories, the corresponding plurality of sequence reads to all or a portion of a reference sequence corresponding to the respective predefined category; determining a count of the number of sequence reads, in the corresponding plurality of sequence reads, that map to a target nucleotide sequence obtained from the corresponding reference sequence; normalizing the count based on the length of the target nucleotide sequence, thus determining the respective normalized read count for the number of sequence reads originating from the respective predefined category; and calculating the amount of the respective predefined category in the sample based on the respective normalized read count, the second normalized read count, and the known quantity of the internal control material.

In some embodiments, the calculating the amount of the respective predefined category in the sample is determined based on the relationship Q_{org}=(Q_{IC}*RC_{org})/RC_{IC}, where Q_{org} is the amount of the respective predefined category in the sample, Q_{IC} is the known quantity of the internal control material, RC_{org} is the respective normalized read count for the number of sequence reads originating from the respective predefined category, and RC_{IC} is the second normalized read count for the number of sequence reads originating from the internal control material.

In some embodiments, the respective normalized read count is expressed as reads per kilobase per million mapped reads (RPKM).

In some embodiments, the respective target nucleotide sequence length, for each respective predefined category in the plurality of predefined categories, is determined from at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of the reference sequence corresponding to the respective predefined category. In some embodiments, the respective target nucleotide sequence length, for each respective predefined category in the plurality of predefined categories, comprises at least two (e.g., at least 2, at least 3, at least 4, at least 5, at least 6, at least 7, at least 8, at least 9, at least 10, at least 15, at least 20, at least 25, at least 30, at least 35, at least 40, at least 45, at least 50, at least 100, at least 200, at least 500, at least 1,000, or more) non-contiguous regions of the reference sequence corresponding to the respective predefined category. In some embodiments, the respective target nucleotide sequence length, for each respective predefined category in the plurality of predefined categories, is determined from a single contiguous region of the reference sequence corresponding to the respective predefined category.

In some embodiments, the respective target nucleotide sequence length, for each respective predefined category in the plurality of predefined categories, comprises at least 50 base pairs or at least 100 base pairs (e.g., contiguous and/or non-contiguous base pairs). In some embodiments, the first target nucleotide sequence length for the first predefined category (e.g., for a first microorganism) and the respective target nucleotide sequence length for a respective predefined category other than the first predefined category (e.g., for a microorganism other than the first microorganism in a plurality of microorganisms) are different.

In some embodiments, the amount of a respective predefined category, in a plurality of predefined categories in the sample, is determined by the relationship Q_{org}=(Q_{IC}*RC_{org})/RC_{IC} and is further corrected by one or more correction factors. In some embodiments, the one or more correction factors comprises an extraction correction factor (e.g., for correcting predefined category-specific differences in extraction efficiency). In some embodiments, the one or more correction factors comprises a sequencing correction factor (e.g., for correcting target-specific differences in sequencing efficiency). In some embodiments, the one or more correction factors comprises an abundance correction factor (e.g., to account for biological differences in abundances of target sequences, such as copy number variations). In some embodiments, the one or more correction factors comprises any one or more of an extraction correction factor, a sequencing correction factor, and/or an abundance correction factor, and/or any combination thereof. In some embodiments, the amount of a respective predefined category, in a plurality of predefined categories, in the sample is corrected based on the relationship Q_{org}=(AF*EF*SF*Q_{IC}*RC_{org})/RC_{IC}, where AF is an abundance correction factor, EF is an extraction correction factor, SF is a sequencing correction factor, Q_{org} is the amount of the respective predefined category in the sample, Q_{IC} is the known quantity of the internal control material, RC_{org} is the respective normalized read count for the number of sequence reads originating from the respective predefined category, and RC_{IC} is the second normalized read count for the number of sequence reads originating from the internal control material.

Other embodiments for the plurality of sequence reads, the reference sequence, the target nucleotide sequence, sequencing, mapping sequence reads, obtaining read counts, normalization, quantification, and any characteristics or elements thereof, for each respective predefined category in a plurality of predefined categories in the sample (e.g., including and/or other than the first predefined category) are possible. For example, any of the embodiments described herein for a plurality of sequence reads, a reference sequence, and a target nucleotide sequence, sequencing, mapping sequence reads, obtaining read counts, normalization, quantification, and any other characteristics or elements thereof, are applicable to a second, third, fourth, fifth, sixth, seventh, eighth, ninth, tenth, and/or any subsequent instances (e.g., for any one or more predefined categories, other than the first predefined category, in a plurality of predefined categories) as to the first instance (e.g., as for a first predefined category in a plurality of predefined categories). Further, any substitutions, modifications, additions, deletions, and/or combinations of any of the systems and methods provided herein are possible, as will be apparent to one skilled in the art.

Another aspect of the present disclosure provides a method for determining, for each sample in a pooled plurality of samples, an amount of a respective predefined category in the respective sample. The method comprises obtaining a plurality of samples, where each sample in the plurality of samples includes one or more nucleic acid molecules originating from a respective predefined category and one or more nucleic acid molecules originating from a respective source other than the predefined category.

The method further comprises adding, to each respective sample in the plurality of samples, a respective known quantity of a respective internal control material comprising one or more nucleic acid molecules. In some embodiments, each respective sample including its respective internal control material, in the plurality of samples, is separately prepared and/or processed for sequencing by any of the methods and/or embodiments disclosed herein.

In some embodiments, the plurality of samples, including their respective internal control materials, are pooled prior to sequencing. In some embodiments, the sequencing is multiplex sequencing. The method subsequently includes obtaining, in electronic form, for each respective sample in the plurality of samples, a respective sequencing dataset comprising a first respective plurality of sequence reads and a second respective plurality of sequence reads from a sequencing of the respective sample including the corresponding internal control material. For each respective sample in the plurality of samples, each sequence read in the first plurality of sequence reads is determined by a sequencing of a nucleic acid molecule in the one or more nucleic acid molecules originating from the respective predefined category, and each sequence read in the second plurality of sequence reads is determined by a sequencing of a nucleic acid molecule in the one or more nucleic acid molecules originating from the respective corresponding internal control material.

In some embodiments, each respective sequencing dataset is isolated based on a unique identifier for the respective sample and its respective corresponding internal control material (e.g., a sequence barcode, unique molecular identifier, adapter sequence, etc.).

For each respective sequencing dataset corresponding to each respective sample in the plurality of samples, the method further comprises determining, from the first respective plurality of sequence reads, a first normalized read count for the number of sequence reads originating from the first predefined category, where the first normalized read count is normalized based on a first target nucleotide sequence length and determining, from the second respective plurality of sequence reads, a second normalized read count for the number of sequence reads originating from the internal control material, where the second normalized read count is normalized based on a second target nucleotide sequence length.

For each respective sequencing dataset corresponding to each respective sample in the plurality of samples, the method includes calculating the amount of the first predefined category in the sample based on the first normalized read count, the second normalized read count, and the known quantity of the internal control material, thus obtaining an amount of a predefined category represented in a sample, for each respective sample in a plurality of samples.

Other embodiments for one or more samples in a plurality of samples, including sample types, sample collection, predefined categories such as organisms and/or microorganisms, sample processing, internal control materials, nucleic acid preparation, sequencing reactions, sequence reads, reference sequences, target nucleotide sequences, mapping sequence reads, obtaining read counts, normalization, quantification, and any characteristics or elements thereof, are possible. For example, any of the embodiments described herein for sample types, sample collection, predefined categories such as organisms and/or microorganisms, sample processing, internal control materials, nucleic acid preparation, sequencing reactions, sequence reads, reference sequences, target nucleotide sequences, mapping sequence reads, obtaining read counts, normalization, quantification, and any other characteristics or elements thereof, are applicable to a second sample and/or a plurality of samples as to a first sample. Further, any substitutions, modifications, additions, deletions, and/or combinations of any of the systems and methods provided herein are possible, as will be apparent to one skilled in the art.

Report Generation.

In some embodiments, the method disclosed herein further comprises generating a report (e.g., a diagnostic report) including the amount of the first predefined category in the sample.

In some embodiments, the report comprises a first therapeutic regimen based on the amount of the first predefined category.

In some embodiments, the first therapeutic regimen is a course of antibiotics, antivirals, antifungals, and/or antiparasitic medication, a combination therapy, and/or a change in diet.

In some embodiments, the first therapeutic regimen is based on the determination that the first predefined category is present in the sample at a concentration above a threshold concentration. For example, in some embodiments, the first predefined category is a pathogenic microorganism, the first therapeutic regimen is selected if the pathogenic microorganism is present in the sample at or above a concentration that is associated with a disease (e.g., a threshold concentration associated with a clinical manifestation of a microorganism), and the first therapeutic regimen is not selected if the pathogenic microorganism is present in the sample below the concentration that is associated with the disease (e.g., the microorganism is present at asymptomatic levels). In some such embodiments, the report further comprises a description and/or an annotation of the pathogen. In some embodiments, the report further comprises a description of the first therapeutic regimen based on the pathogen. In some embodiments, the report further comprises an annotation of the first therapeutic regimen based on clinical and/or health data.

In some embodiments, sample is a clinical sample from a patient undergoing a therapy, and the first therapeutic regimen comprises a change from a current therapy to a new therapy. For example, in some embodiments, the first therapeutic regimen is selected if the pathogenic microorganism is present in the sample at a concentration that indicates an undesirable effect of the current therapy (e.g., lack of efficacy and/or change of efficacy due to antimicrobial resistance).

In some embodiments, the report comprises an antimicrobial resistance status for the first predefined category (e.g., where the first predefined category is a first organism and/or microorganism), and the first therapeutic regimen is based on the amount of the first predefined category and the antimicrobial resistance status for the first predefined category.

For example, in some embodiments, the first predefined category is a pathogenic microorganism comprising an antimicrobial resistance gene, the first therapeutic regimen is selected for the pathogen with the antimicrobial resistance gene if the pathogenic microorganism is present in the sample at or above a concentration that is associated with a disease (e.g., a threshold concentration associated with a clinical manifestation of a microorganism), and the first therapeutic regimen is not selected if the pathogenic microorganism is present in the sample below the concentration that is associated with the disease (e.g., the microorganism is present at asymptomatic levels).

In some embodiments, quantification of one or more antimicrobial resistance genes is used to direct the use of one or more respective antimicrobial medicines or combinatorial therapeutics. For example, in some cases, quantification is used to select a treatment that attenuates or eliminates the expression or protein activity of the antimicrobial resistance gene (e.g., by antisense RNA, RNA interference (RNAi) sequences, antibodies, or small molecule inhibitors).

In some embodiments, the report further comprises a description and/or an annotation of the antimicrobial resistance gene.

In some embodiments, the report further comprises a patient status, such as a patient response status. For example, in some embodiments, the report includes a status of a patient that is undergoing monitoring in response to a treatment. In some embodiments, the patient response status is a change in an amount of a predefined category in a sample from the patient (e.g., an organism, microorganism, cell type, cell origin, and/or other population) after administration of a therapeutic regimen. In some embodiments, the report includes a determination of an efficacy of a treatment, based at least in part on the patient response status.

In some embodiments, the report further comprises an amount of a second predefined category in the sample, calculated based on a normalized read count for the second predefined category, the second normalized read count for the internal control material, and the known quantity of the internal control material. In some embodiments, the report further comprises a second therapeutic regimen based on the amount of the second predefined category. In some embodiments, the report comprises an antimicrobial resistance status for the second predefined category, and the second therapeutic regimen is based on the amount of the second predefined category and the antimicrobial resistance status for the second predefined category.

In some embodiments, the generating of a report comprises transmitting the report to a cloud computing infrastructure (e.g., an email). In some embodiments, the report is generated as an email that can be sent to, for example, a patient, a medical practitioner (e.g., a primary physician), a hospital and/or a diagnostic laboratory. In some embodiments, the report is stored for retrieval. In some embodiments, the report is transmitted to a cloud computing infrastructure (e.g., a server) for storage. In some embodiments, the report is generated in a printable format. In some embodiments, the report is generated as a printable document (e.g., a PDF).

Additional embodiments, substitutions, modifications, additions, deletions, and/or combinations of any of the systems and methods provided herein are possible, as will be apparent to one skilled in the art. See, for example, IDbyDNA, 2019, “Explify Software v1.5.0 User Manual,” Document No. TH-2019-200-006, pp. 1-44, which is hereby incorporated by reference herein in its entirety.

### Additional Embodiments

Another aspect of the present disclosure provides a computer system having one or more processors, and memory storing one or more programs for execution by the one or more processors, the one or more programs comprising instructions for determining an amount of a first predefined category in a sample. The one or more programs comprise instructions for obtaining a sample including (i) one or more nucleic acid molecules originating from the first predefined category and (ii) one or more nucleic acid molecules originating from a source other than the first predefined category, and adding to the sample a known quantity of an internal control material comprising one or more nucleic acid molecules. The one or more programs further comprise obtaining, in electronic form, a sequencing dataset comprising a first plurality of sequence reads and a second plurality of sequence reads from a sequencing of the sample including the internal control material, where each respective sequence read in the first plurality of sequence reads is determined by a sequencing of a nucleic acid molecule in the one or more nucleic acid molecules originating from the first predefined category, and each respective sequence read in the second plurality of sequence reads is determined by a sequencing of a nucleic acid molecule in the one or more nucleic acid molecules originating from the internal control material. The one or more programs further comprise determining, from the first plurality of sequence reads, a first normalized read count for the number of sequence reads originating from the first predefined category, where the first normalized read count is normalized based on a first target nucleotide sequence length, and determining, from the second plurality of sequence reads, a second normalized read count for the number of sequence reads originating from the internal control material, where the second normalized read count is normalized based on a second target nucleotide sequence length. The one or more programs further comprise calculating the amount of the first predefined category in the sample based on the first normalized read count, the second normalized read count, and the known quantity of the internal control material.

Another aspect of the present disclosure provides a non-transitory computer readable storage medium storing one or more programs configured for execution by a computer, the one or more programs comprising instructions for determining an amount of a first predefined category in a sample. The one or more programs comprise instructions for obtaining a sample including (i) one or more nucleic acid molecules originating from the first predefined category and (ii) one or more nucleic acid molecules originating from a source other than the first predefined category, and adding to the sample a known quantity of an internal control material comprising one or more nucleic acid molecules. The one or more programs further comprise obtaining, in electronic form, a sequencing dataset comprising a first plurality of sequence reads and a second plurality of sequence reads from a sequencing of the sample including the internal control material, where each respective sequence read in the first plurality of sequence reads is determined by a sequencing of a nucleic acid molecule in the one or more nucleic acid molecules originating from the first predefined category, and each respective sequence read in the second plurality of sequence reads is determined by a sequencing of a nucleic acid molecule in the one or more nucleic acid molecules originating from the internal control material. The one or more programs further comprise determining, from the first plurality of sequence reads, a first normalized read count for the number of sequence reads originating from the first predefined category, where the first normalized read count is normalized based on a first target nucleotide sequence length, and determining, from the second plurality of sequence reads, a second normalized read count for the number of sequence reads originating from the internal control material, where the second normalized read count is normalized based on a second target nucleotide sequence length. The one or more programs further comprise calculating the amount of the first predefined category in the sample based on the first normalized read count, the second normalized read count, and the known quantity of the internal control material.

Another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors.

Another aspect of the present disclosure provides a non-transitory computer readable storage medium storing one or more programs configured for execution by a computer, the one or more programs comprising instructions for carrying out any of the methods disclosed herein.

### EXAMPLES

In some embodiments, the systems and methods described herein are useful for a variety of applications including, but not limited to, metagenomics, cancer diagnostics, human variation (pharmacogenomics and ancestry), and agricultural and food analysis. In some embodiments, the systems and methods described herein are useful for bacterial and fungal classification, viral classification, parasite classification, human mRNA transcript profiling, identification of infection and contamination, detection and/or quantification of microorganisms for, e.g., education, consumers, food safety and authenticity, hospital safety and contamination monitoring, biological product quality and safety monitoring, animal disease diagnostics and treatment, microbial strain profiling, tumor profiling, forensic profiling, and/or genetic testing.

### Example 1—Explify Software Platform

In some embodiments, information about a biological sample, such as information regarding quantification of one or more predefined categories in the sample, are presented using a software program or platform. The software platform can include one or more components, such as a component for providing information about a sample, a component for analyzing sequencing information (e.g., performing a k-mer based analysis), a component for analyzing and classifying processed sequencing reads, and a component for supporting laboratory sample preparation. The Explify Software Platform (e.g., Software v1.5.0) is an exemplary platform that includes three such components: the Explify ReviewPortal, which is a web browser-accessible dashboard application; the Explify Analysis Pipeline, which processes raw NGS data for analysis by the Explify Classification Algorithm; and the Explify SeqPortal web-based application (also called Workflow Manager), which supports sample information entry and laboratory sample preparation. See, for example, IDbyDNA, 2019, “Explify Software v1.5.0 User Manual,” Document No. TH-2019-200-006, pp. 1-44, which is hereby incorporated by reference herein in its entirety.

### Example 2: Example Workflow

FIG. 3 illustrates an example workflow for processing biological samples for quantification of predefined categories, in accordance with some embodiments of the present disclosure. In Block 300, samples are collected (e.g., as described herein). In some embodiments, samples are collected from biological sources including, but not limited to, human subjects, environmental sources, industrial sources, and/or other sources. In some embodiments, samples include fluids and/or solids. In some embodiments, samples are processed to prepare the samples for subsequent sequencing (310). Optionally, samples are divided into two or more portions for subsequent analysis, where samples to be analyzed for nucleic acids included therein are processed and/or analyzed separately from samples to be analyzed for alternative analytes (e.g., polypeptides (330)) included therein. In some embodiments, sequences of nucleic acid molecules of the sample are analyzed using nucleic acid sequencing techniques (320). Data prepared from this analysis, including sequencing reads, is collected and optionally combined. In some embodiments, data is stored locally and/or in a web- or cloud-based storage system. In some embodiments, data is compared against sequences in one or more reference databases (e.g., as described herein) (340), and/or is processed and interpreted using a software program, such as a web-based software program. In some embodiments, a user prepares and/or interprets various representations of the data. In some embodiments, the data is analyzed to interpret the nucleic acid molecules included in the sample, thus identifying predefined categories (e.g., microorganisms, viruses, genes, or other contents of the sample) (350). A variety of representations of the data can be prepared (e.g., as described herein). Such representations and reports are used, in some instances, to inform a variety of interventions including medical interventions and physical interventions (e.g., as described herein). For example, a report can be used to inform a treatment regimen for a patient.

### Example 3: Performance Measures Using Known Pathogen Concentrations

FIGS. 4A, 4B, and 4C illustrate comparisons of known pathogen concentrations in example specimens to calculated concentrations, in accordance with some embodiments of the present disclosure.

To demonstrate the utility of the absolute quantification approach disclosed herein, a titration of the ZymoBIOMICS Molecular Community Standard (MCS) was combined with a fixed known concentration of internal control material and processed for next-generation sequencing. The ZymoBIOMICS Microbial Community Standard is the first commercially available standard for microbiomics and metagenomics studies. The microbial standard is a well-defined, accurately characterized mock community consisting of Gram-negative and Gram-positive bacteria and yeast with varying sizes and cell wall composition. The wide range of organisms with different properties enables characterization, optimization, and validation of lysis methods such as bead beating. It can be used as a defined input to assess the performance of entire microbiomic/metagenomic workflows, therefore enabling workflows to be optimized and validated. A mock microbial DNA community standard allows researchers to focus the optimization after the step of DNA extraction. See, for example, Nicholls et al., 2019, “Ultra-deep, long-read nanopore sequencing of mock microbial community standards,” GigaScience 8(5), giz043; doi: 10.1093/gigascience/giz043.

The MCS contains a known concentration of the pathogens Staphylococcus aureus and Enterococcus faecalis, such that the expected concentration of these pathogens and the IC material in the titration samples are as provided in Table 4. Titration samples included 10-fold serial dilutions at 1:1, 1:10, 1:100, 1:1000, and 1:10,000 for each of S. aureus and E. faecalis. All titrations were prepared in triplicate. To each replicate of each titration sample, a constant amount of IC material was added (3×10⁶ genomic equivalents (GE)/mL).

TABLE 4
Known Concentrations of Pathogens and IC Material

| | S. aureus | E. faecalis | IC |
|---|---|---|---|
| Titration/Replicate | (GE/mL) | (GE/mL) | (GE/mL) |
| 1:1 (Rep 1) | 2.13 × 10⁹ | 2.04 × 10⁹ | 3 × 10⁶ |
| 1:1 (Rep 2) | 2.13 × 10⁹ | 2.04 × 10⁹ | 3 × 10⁶ |
| 1:1 (Rep 3) | 2.13 × 10⁹ | 2.04 × 10⁹ | 3 × 10⁶ |
| 1:10 (Rep 1) | 2.13 × 10⁸ | 2.04 × 10⁸ | 3 × 10⁶ |
| 1:10 (Rep 2) | 2.13 × 10⁸ | 2.04 × 10⁸ | 3 × 10⁶ |
| 1:10 (Rep 3) | 2.13 × 10⁸ | 2.04 × 10⁸ | 3 × 10⁶ |
| 1:100 (Rep 1) | 2.13 × 10⁷ | 2.04 × 10⁷ | 3 × 10⁶ |
| 1:100 (Rep 2) | 2.13 × 10⁷ | 2.04 × 10⁷ | 3 × 10⁶ |
| 1:100 (Rep 3) | 2.13 × 10⁷ | 2.04 × 10⁷ | 3 × 10⁶ |
| 1:1000 (Rep 1) | 2.13 × 10⁶ | 2.04 × 10⁶ | 3 × 10⁶ |
| 1:1000 (Rep 2) | 2.13 × 10⁶ | 2.04 × 10⁶ | 3 × 10⁶ |
| 1:1000 (Rep 3) | 2.13 × 10⁶ | 2.04 × 10⁶ | 3 × 10⁶ |
| 1:10,000 (Rep 1) | 2.13 × 10⁵ | 2.04 × 10⁵ | 3 × 10⁶ |
| 1:10,000 (Rep 2) | 2.13 × 10⁵ | 2.04 × 10⁵ | 3 × 10⁶ |
| 1:10,000 (Rep 3) | 2.13 × 10⁵ | 2.04 × 10⁵ | 3 × 10⁶ |

MCS standard samples including IC material at the dilutions and concentrations listed above were sequenced using next-generation sequencing, and read counts were normalized in accordance with an embodiment of the present disclosure. Normalized read counts were calculated as reads per kilobase per million mapped reads (RPKM) according to the formula RPKM=(number of reads mapped to target×10³×10⁶)/(total number of reads×target length in bp), where targets were identified separately using a reference sequence (e.g., genome) of S. aureus, E. faecalis, and IC material. The constant values 10³ and 10⁶ were used to normalize for gene length and sequencing depth factor, respectively. Normalized read counts for S. aureus, E. faecalis, and IC material in all titrations were calculated as provided in Table 5.

TABLE 5
Normalized Read Counts for Pathogen Titrations and IC Material

| | S. aureus | E. faecalis | IC |
|---|---|---|---|
| Titration/Replicate | (RPKM) | (RPKM) | (RPKM) |
| 1:1 (Rep 1) | 3.54 × 10⁴ | 2.21 × 10⁴ | 1.39 × 10² |
| 1:1 (Rep 2) | 3.54 × 10⁴ | 2.01 × 10⁴ | 1.31 × 10² |
| 1:1 (Rep 3) | 4.06 × 10⁴ | 3.49 × 10⁴ | 1.09 × 10² |
| 1:10 (Rep 1) | 3.66 × 10⁴ | 2.37 × 10⁴ | 7.75 × 10² |
| 1:10 (Rep 2) | 3.58 × 10⁴ | 2.79 × 10⁴ | 8.62 × 10² |
| 1:10 (Rep 3) | 4.24 × 10⁴ | 3.80 × 10⁴ | 5.34 × 10² |
| 1:100 (Rep 1) | 3.68 × 10⁴ | 3.42 × 10⁴ | 7.57 × 10³ |
| 1:100 (Rep 2) | 3.53 × 10⁴ | 2.89 × 10⁴ | 8.33 × 10³ |
| 1:1000 (Rep 1) | 3.29 × 10⁴ | 2.47 × 10⁴ | 7.30 × 10⁴ |
| 1:1000 (Rep 2) | 3.43 × 10⁴ | 2.94 × 10⁴ | 6.86 × 10⁴ |
| 1:1000 (Rep 3) | 4.58 × 10⁴ | 3.55 × 10⁴ | 3.94 × 10⁴ |
| 1:10,000 (Rep 1) | 1.92 × 10⁴ | 1.51 × 10⁴ | 3.55 × 10⁵ |
| 1:10,000 (Rep 2) | 2.00 × 10⁴ | 1.67 × 10⁴ | 3.55 × 10⁵ |
| 1:10,000 (Rep 3) | 3.03 × 10⁴ | 2.45 × 10⁴ | 2.45 × 10⁵ |

The normalized read counts for Staphylococcus aureus, Enterococcus faecalis, and the IC material, along with the fixed known concentration of IC material, were applied to the ratio equation Q_{org}=(Q_{IC}*RC_{org})/RC_{IC}, where Q_{org} is the unknown amount of each pathogen (e.g., S. aureus and E. faecalis) in the sample, Q_{IC} is the known quantity of the internal control material, RC_{org} is the normalized read count (e.g., RPKM) for the number of sequence reads originating from the pathogen, and RC_{IC} is the second normalized read count (e.g., RPKM) for the number of sequence reads originating from the internal control material, in accordance with an embodiment of the present disclosure. Solving for Q_{org} using the ratio equation, the concentrations of Staphylococcus aureus and Enterococcus faecalis in the MCS titration samples were calculated, as shown in Table 6. For example, the concentration of E. faecalis for replicate 1 of the 1:1 titration can be calculated as follows: (3.00×10⁶)×(2.2×10⁴)/(1.39×10²)=4.77×10⁸, using the above values for Q_{IC}, RC_{org}, and RC_{IC} in Tables 4 and 5.

TABLE 6
Calculated Concentrations of Pathogens

| Titration/Replicate | S. aureus (GE/mL) | E. faecalis (GE/mL) |
|---|---|---|
| 1:1 (Rep 1) | 7.64 × 10⁸ | 4.77 × 10⁸ |
| 1:1 (Rep 2) | 8.10 × 10⁸ | 4.59 × 10⁸ |
| 1:1 (Rep 3) | 1.12 × 10⁹ | 9.61 × 10⁸ |
| 1:10 (Rep 1) | 1.42 × 10⁸ | 9.17 × 10⁷ |
| 1:10 (Rep 2) | 1.25 × 10⁸ | 9.69 × 10⁷ |
| 1:10 (Rep 3) | 2.39 × 10⁸ | 2.14 × 10⁸ |
| 1:100 (Rep 1) | 1.46 × 10⁷ | 1.35 × 10⁷ |
| 1:100 (Rep 2) | 1.27 × 10⁷ | 1.04 × 10⁷ |
| 1:1000 (Rep 1) | 1.35 × 10⁶ | 1.02 × 10⁶ |
| 1:1000 (Rep 2) | 1.50 × 10⁶ | 1.28 × 10⁶ |
| 1:1000 (Rep 3) | 3.49 × 10⁶ | 2.70 × 10⁶ |
| 1:10,000 (Rep 1) | 1.62 × 10⁵ | 1.27 × 10⁵ |
| 1:10,000 (Rep 2) | 1.69 × 10⁵ | 1.41 × 10⁵ |
| 1:10,000 (Rep 3) | 3.70 × 10⁵ | 2.99 × 10⁵ |

A comparison between the calculated concentrations for Staphylococcus aureus and Enterococcus faecalis listed in Table 6 (e.g., using the presently disclosed methods) and the known concentrations for the same listed in Table 4 (e.g., obtained from the ZymoBIOMICS Microbial Community Standard), reveals excellent agreement between the calculated and known concentrations in all titration samples. This concordance is further illustrated in FIGS. 4A (Staphylococcus aureus) and 4B (Enterococcus faecalis), where the known concentrations are plotted against the calculated concentrations and show a high correlation between the predicted and actual values (R-squared >0.98). In FIGS. 4A and 4B, experimental data points are indicated by black squares and trend lines are indicated as solid black lines.

Another performance measure for the quantification methods provided herein is illustrated in FIG. 4C. A cohort of clinical respiratory tract specimens was obtained and assayed using the Centers for Disease Control and Prevention (CDC) quantitative PCR (qPCR) SARS-CoV-2 assay. The CDC qPCR SARS-CoV-2 assay provided viral loads (VL) of SARS-CoV-2 for the specimens. For comparison, internal control material was added to the clinical respiratory tract specimens and the concentration (GE/mL) was calculated after sample processing and sequencing, in accordance with an embodiment of the present disclosure. High concordance between the calculated concentration (VL Ratio) and the actual concentration obtained from qPCR (VL qPCR) is shown by the graph in FIG. 4C, which plots VL Ratio against VL qPCR. The results illustrate that the internal control methods provided herein exhibit comparable accuracy in quantification compared to more laborious, template-specific methods such as qPCR.

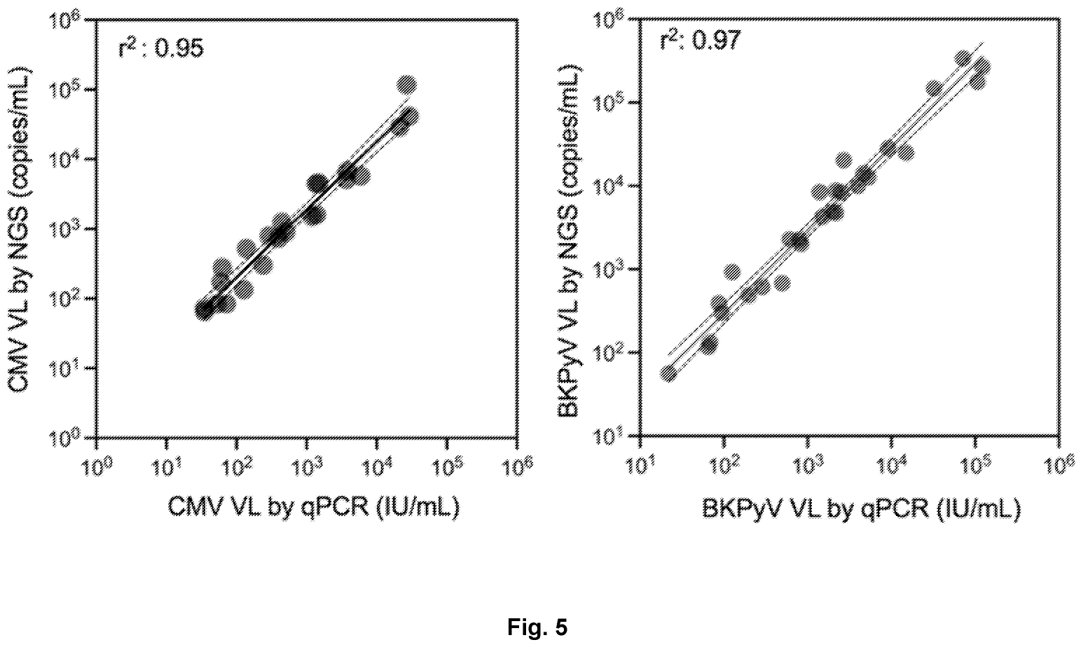

Other performance measures for the quantification methods provided herein are illustrated in FIG. 5. Plasma samples were obtained from subjects infected with cytomegalovirus (CMV; left panel) and BK polyomavirus (BKPyV; right panel) and used to generate sequencing datasets using next-generation sequencing. Viral load (VL) was determined for the plasma samples in accordance with an embodiment of the present disclosure. Correlations between the calculated plasma viral loads and expected viral loads obtained using quantitative PCR (qPCR) showed high concordance between the presently disclosed methods and expected values, further illustrating that the internal control methods provided herein exhibit comparable accuracy in quantification compared to more laborious, template-specific methods such as qPCR.

### Example 4: Correction of Quantification

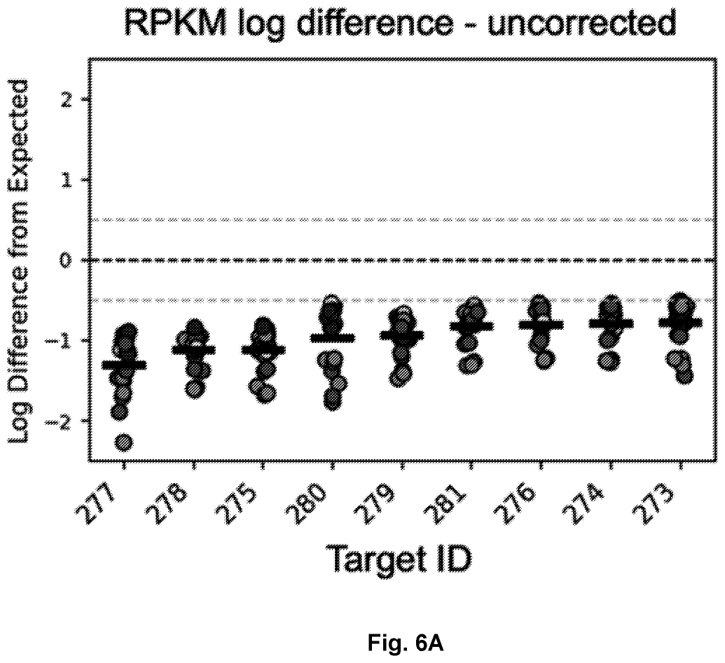

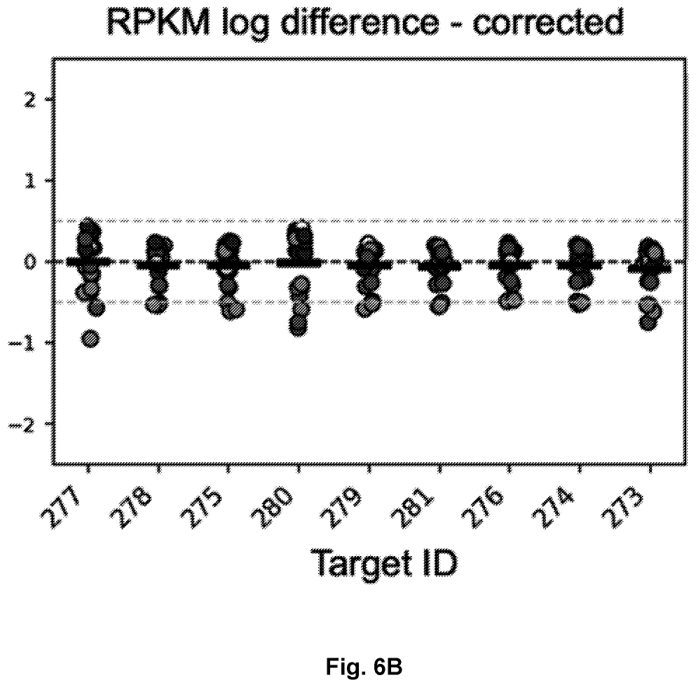

Quantification of a plurality of target nucleotide sequences for an example organism was compared without (FIG. 6A) and with (FIG. 6B) correction using application of one or more correction factors, in accordance with an embodiment of the present disclosure. The RPKM log difference between the calculated amount and the expected amount of each of the organism's target nucleotide sequences (277, 278, . . . 273) showed a disparity between the calculated and expected amounts without correction. Conversely, after application of correction factors, the log difference between the calculated and expected amounts were decreased such that calculated quantification matched expected quantification. These results illustrate the effectiveness in applying correction factors for accurate quantification of predefined categories (e.g., organisms) in samples.

### CONCLUSION

All references cited herein are incorporated herein by reference in their entirety and for all purposes to the same extent as if each individual publication or patent or patent application was specifically and individually indicated to be incorporated by reference in its entirety for all purposes.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A method for determining an amount of a first predefined category represented in a sample, comprising:
obtaining a sample including (i) one or more nucleic acid molecules originating from the first predefined category and (ii) one or more nucleic acid molecules originating from a source other than the first predefined category;
adding to the sample a known quantity of an internal control material comprising one or more nucleic acid molecules;
obtaining, in electronic form, a sequencing dataset comprising a first plurality of sequence reads and a second plurality of sequence reads from a sequencing of the sample including the internal control material, wherein:
each respective sequence read in the first plurality of sequence reads is determined by a sequencing of a nucleic acid molecule in the one or more nucleic acid molecules originating from the first predefined category, and
each respective sequence read in the second plurality of sequence reads is determined by a sequencing of a nucleic acid molecule in the one or more nucleic acid molecules originating from the internal control material;
determining, from the first plurality of sequence reads, a first normalized read count for the number of sequence reads originating from the first predefined category, wherein the first normalized read count is normalized based on a first target nucleotide sequence length;
determining, from the second plurality of sequence reads, a second normalized read count for the number of sequence reads originating from the internal control material, wherein the second normalized read count is normalized based on a second target nucleotide sequence length; and
calculating the amount of the first predefined category represented in the sample based on the first normalized read count, the second normalized read count, and the known quantity of the internal control material.

2. The method of claim 1, wherein the calculating the amount of the first predefined category in the sample is determined by dividing the product of (i) the known quantity of the internal control material and (ii) the first normalized read count by the second normalized read count.

3. The method of claim 1 or 2, further comprising correcting the amount of the first predefined category in the sample using an extraction correction factor.

4. The method of claim 3, wherein the extraction correction factor is obtained based on a sequencing of a known amount of one or more extraction correction sequences in a plurality of extraction correction sequences.

5. The method of claim 4, wherein an extraction correction sequence in the plurality of extraction correction sequences comprises all or a portion of a reference sequence corresponding to a predefined category in a plurality of predefined categories.

6. The method of claim 4 or 5, wherein the plurality of extraction correction sequences comprises all or a portion of a first reference sequence corresponding to the first predefined category.

7. The method of any one of claims 3-6, wherein the extraction correction factor is a fixed value.

8. The method of any one of claims 1-7, further comprising correcting the amount of the first predefined category in the sample using a sequencing correction factor.

9. The method of claim 8, wherein the sequencing correction factor is obtained based on a sequencing of a known amount of one or more sequencing-correction sequences in a plurality of sequencing-correction sequences.

10. The method of claim 9, wherein a sequencing-correction sequence in the plurality of sequencing-correction sequences comprises all or a portion of a reference sequence corresponding to a predefined category in a plurality of predefined categories.

11. The method of claim 9 or 10, wherein the plurality of sequencing-correction sequences comprises all or a portion of a first target nucleotide sequence corresponding to the first predefined category.

12. The method of any one of claims 8-11, wherein the sequencing correction factor is a fixed value.

13. The method of any one of claims 1-12, further comprising correcting the amount of the first predefined category in the sample using an abundance correction factor.

14. The method of any one of claims 1-13, wherein the calculating the amount of the first predefined category represented in the sample is calculated as the product of (i) an abundance correction factor, (ii) an extraction correction factor, (iii) a sequencing correction factor, (iv) the known quantity of the internal control material, and (v) the first normalized read count divided by the second normalized read count.

15. The method of any one of claims 1-14, wherein the determining the first read count and the second read count further comprises:
mapping the first plurality of sequence reads to all or a portion of a first reference sequence corresponding to the first predefined category; and
mapping the second plurality of sequence reads to all or a portion of a second reference sequence corresponding to the internal control material.

16. The method of claim 15, further comprising:
determining a first count of the number of sequence reads, in the first plurality of sequence reads, that map to a first target nucleotide sequence obtained from the first reference sequence corresponding to the first predefined category;
determining a second count of the number of sequence reads, in the second plurality of sequence reads, that map to a second target nucleotide sequence obtained from the second reference sequence corresponding to the internal control material;
normalizing the first count based on the length of the first target nucleotide sequence; and
normalizing the second count based on the length of the second target nucleotide sequence, thereby obtaining the first normalized read count and the second normalized read count, respectively.

17. The method of any one of claims 1-16, wherein the first normalized read count and the second normalized read count are expressed as reads per kilobase per million mapped reads (RPKM).

18. The method of any one of claims 1-17, wherein the first target nucleotide sequence length is determined from all or a portion of a reference sequence corresponding to the first predefined category.

19. The method of any one of claims 1-18, wherein the first target nucleotide sequence length is determined from at least two non-contiguous regions of a reference sequence corresponding to the first predefined category.

20. The method of any one of claims 1-18, wherein the first target nucleotide sequence length is determined from a single contiguous region of a reference sequence corresponding to the first predefined category.

21. The method of any one of claims 1-20, wherein the first target nucleotide sequence length comprises at least 50 base pairs.

22. The method of any one of claims 1-21, wherein the first target nucleotide sequence length and the second target nucleotide sequence length are different.

23. The method of any one of claims 1-22, wherein the second target nucleotide sequence length is determined from all or a portion of a reference sequence corresponding to the internal control material.

24. The method of any one of claims 1-23, wherein the second target nucleotide sequence length is determined from at least two non-contiguous regions of a reference sequence corresponding to the internal control material.

25. The method of any one of claims 1-23, wherein the second target nucleotide sequence length is determined from a single contiguous region of a reference sequence corresponding to the internal control material.

26. The method of any one of claims 1-25, wherein the second target nucleotide sequence length comprises at least 50 base pairs.

27. The method of any one of claims 1-26, wherein the sequencing reaction is a whole transcriptome sequencing reaction.

28. The method of any one of claims 1-26, wherein the sequencing reaction is a whole genome sequencing reaction.

29. The method of any one of claims 1-28, wherein the sequencing dataset comprises at least 1×10³, at least 1×10⁴, at least 1×10⁵, at least 1×10⁶, at least 1×10⁷, at least 1×10⁸, or at least 2×10⁸ sequence reads.

30. The method of any one of claims 1-29, wherein the first plurality of sequence reads collectively maps to at least 50 base pairs or at least 100 base pairs of a first reference sequence corresponding to the first predefined category.

31. The method of any one of claims 1-30, wherein the second plurality of sequence reads collectively maps to at least 50 base pairs of a second reference sequence corresponding to the internal control material.

32. The method of any one of claims 1-31, wherein the sample is obtained from a biological subject.

33. The method of any one of claims 1-32, wherein the sample is obtained from a human with a disease condition.

34. The method of any one of claims 1-33, wherein the first predefined category is a microorganism.

35. The method of claim 34, wherein the microorganism is selected from the group consisting of bacterial, fungal, viral, and parasitic.

36. The method of claim 34 or 35, wherein the microorganism is a pathogen.

37. The method of any one of claims 1-36, wherein the source other than the first predefined category is human.

38. The method of any one of claims 1-37, wherein the sequencing dataset further includes a third plurality of sequence reads, wherein each respective sequence read in the third plurality of sequence reads is determined by a sequencing of a nucleic acid molecule in the one or more nucleic acid molecules originating from the source other than the first predefined category.

39. The method of claim 38, further comprising:
mapping the third plurality of sequence reads to all or a portion of a third reference sequence corresponding to the source other than the first predefined category;
determining a third count of the number of sequence reads, in the third plurality of sequence reads, that map to a third target nucleotide sequence obtained from the third reference sequence corresponding to the source other than the first predefined category;
normalizing the third count based on the length of the third target nucleotide sequence, thereby determining a third normalized read count for the number of sequence reads originating from the source other than the first predefined category; and
calculating the amount of the first predefined category in the sample based at least in part on the third normalized read count.

40. The method of claim 39, wherein the third normalized read count is expressed as reads per kilobase per million mapped reads (RPKM).

41. The method of claim 39 or 40, wherein the third target nucleotide sequence length is determined from at least two non-contiguous regions of the third reference sequence corresponding to the source other than the first predefined category.

42. The method of claim 39 or 40, wherein the third target nucleotide sequence length is determined from a single contiguous region of the third reference sequence corresponding to the source other than the first predefined category.

43. The method of any one of claims 38-42, wherein the third plurality of sequence reads collectively maps to at least 50 base pairs of a third reference sequence corresponding to the source other than the first predefined category.

44. The method of any one of claims 1-43, wherein:
the first predefined category is in a plurality of predefined categories in the sample and the dataset comprises a corresponding plurality of sequence reads, for each predefined category in the plurality of predefined categories, including the first plurality of sequence reads for the first predefined category, and
the method further comprises, for each respective predefined category beyond the first predefined category in the plurality of predefined categories:
determining a respective normalized read count for the number of sequence reads originating from the respective predefined category, wherein the respective normalized read count is normalized based on a corresponding target nucleotide sequence length for the respective predefined category, and
calculating the amount of the respective predefined category in the sample based on the respective normalized read count for the number of sequence reads originating from the respective predefined category, the second normalized read count, and the known quantity of the internal control material.

45. The method of claim 44, further comprising:
mapping, for each respective predefined category beyond the first predefined category in the plurality of predefined categories, the corresponding plurality of sequence reads to all or a portion of a reference sequence corresponding to the respective predefined category;
determining a count of the number of sequence reads, in the corresponding plurality of sequence reads, that map to a target nucleotide sequence obtained from the corresponding reference sequence;
normalizing the count based on the length of the target nucleotide sequence, thereby determining the respective normalized read count for the number of sequence reads originating from the respective predefined category; and
calculating the amount of the respective predefined category in the sample based on the respective normalized read count, the second normalized read count, and the known quantity of the internal control material.

46. The method of claim 45, wherein the calculating the amount of the respective predefined category in the sample is determined by dividing the product of (i) the known quantity of the internal control material and (ii) the respective normalized read count for the number of sequence reads originating from the respective predefined category by the second normalized read count for the number of sequence reads originating from the internal control material.

47. The method of claim 45, wherein the calculating the amount of the respective predefined category in the sample is determined by dividing the product of (i) an abundance correction factor, (ii) an extraction correction factor, (iii) a sequencing correction factor, (iv) the known quantity of the internal control material, and (v) the respective normalized read count for the number of sequence reads originating from the respective predefined category by the second normalized read count for the number of sequence reads originating from the internal control material.

48. The method of any one of claims 45-47, wherein the respective normalized read count is expressed as reads per kilobase per million mapped reads (RPKM).

49. The method of any one of claims 45-48, wherein the respective target nucleotide sequence length is determined from at least two non-contiguous regions of the reference sequence corresponding to the respective predefined category.

50. The method of any one of claims 45-48, wherein the respective target nucleotide sequence length is determined from a single contiguous region of the reference sequence corresponding to the respective predefined category.

51. The method of any one of claims 45-50, wherein the respective target nucleotide sequence length comprises at least 50 base pairs.

52. The method of any one of claims 45-51, wherein the first target nucleotide sequence length for the first predefined category and the respective target nucleotide sequence length, for the respective predefined category beyond the first predefined category in the plurality of predefined categories, are different.

53. The method of any one of claims 44-52, wherein the respective predefined category is a microorganism.

54. The method of claim 53, wherein the microorganism is selected from the group consisting of bacterial, fungal, viral, and parasitic.

55. The method of claim 53 or 54, wherein the microorganism is a pathogen.

56. The method of any one of claims 44-55, wherein the respective plurality of sequence reads collectively maps to at least 50 base pairs of a reference sequence corresponding to the respective predefined category.

57. The method of any one of claims 44-56, wherein the amount of the first predefined category in the sample and the amount of the respective predefined category other than the first predefined category in the plurality of predefined categories, in the sample are different.

58. The method of any one of claims 1-57, further comprising generating a report including the amount of the first predefined category in the sample.

59. The method of claim 58, wherein the report comprises a first therapeutic regimen based on the amount of the first predefined category.

60. The method of claim 59, wherein the first predefined category is a first organism, the report comprises an antimicrobial resistance status for the first organism, and the first therapeutic regimen is based on the amount of the first organism and the antimicrobial resistance status for the first organism.

61. The method of any one of claims 58-60, wherein the report comprises a patient status.

62. The method of any one of claims 58-61, wherein the first predefined category is in a plurality of predefined categories in the sample, and the report further comprises, for each respective predefined category beyond the first predefined category in the plurality of predefined categories, an amount of the respective predefined category in the sample, calculated based on a respective normalized read count for the respective predefined category, the second normalized read count for the internal control material, and the known quantity of the internal control material.

63. The method of any one of claims 58-62, wherein the generating a report comprises transmitting the report to a cloud computing infrastructure.

64. A non-transitory computer-readable storage medium having stored thereon program code instructions that, when executed by a processor, cause the processor to perform a method for determining an amount of a first predefined category represented in a sample, the method comprising:
obtaining, in electronic form, a sequencing dataset comprising a first plurality of sequence reads and a second plurality of sequence reads originating from a sequencing of the sample, wherein the sample comprises (i) a plurality of nucleic acid molecules originating from the first predefined category, (ii) a plurality of nucleic acid molecules originating from a source other than the first predefined category, and (iii) a known quantity of an internal control material comprising one or more nucleic acid molecules, wherein:
each respective sequence read in the first plurality of sequence reads is determined by a sequencing of a nucleic acid molecule in the plurality of nucleic acid molecules originating from the first predefined category, and
each respective sequence read in the second plurality of sequence reads is determined by a sequencing of a nucleic acid molecule in the plurality of nucleic acid molecules originating from the internal control material;
determining, from the first plurality of sequence reads, a first normalized read count for the number of sequence reads originating from the first predefined category, wherein the first normalized read count is normalized based on a first target nucleotide sequence length;
determining, from the second plurality of sequence reads, a second normalized read count for the number of sequence reads originating from the internal control material, wherein the second normalized read count is normalized based on a second target nucleotide sequence length; and
calculating the amount of the first predefined category in the sample based on the first normalized read count, the second normalized read count, and the known quantity of the internal control material.

65. The non-transitory computer-readable storage medium of claim 64, wherein the calculating the amount of the first predefined category in the sample is determined by dividing the product of (i) the known quantity of the internal control material and (ii) the first normalized read count by the second normalized read count.

66. The non-transitory computer-readable storage medium of claim 64 or 65, the method further comprising correcting the amount of the first predefined category in the sample using an extraction correction factor.

67. The non-transitory computer-readable storage medium of claim 66, wherein the extraction correction factor is obtained based on a sequencing of a known amount of one or more extraction correction sequences in a plurality of extraction correction sequences.

68. The non-transitory computer-readable storage medium of claim 67, wherein an extraction correction sequence in the plurality of extraction correction sequences comprises all or a portion of a reference sequence corresponding to a predefined category in a plurality of predefined categories.

69. The non-transitory computer-readable storage medium of claim 67 or 68, wherein the plurality of extraction correction sequences comprises all or a portion of a first reference sequence corresponding to the first predefined category.

70. The non-transitory computer-readable storage medium of any one of claims 66-69, wherein the extraction correction factor is a fixed value.

71. The non-transitory computer-readable storage medium of any one of claims 64-70, further comprising correcting the amount of the first predefined category in the sample using a sequencing correction factor.

72. The non-transitory computer-readable storage medium of claim 71, wherein the sequencing correction factor is obtained based on a sequencing of a known amount of one or more sequencing-correction sequences in a plurality of sequencing-correction sequences.

73. The non-transitory computer-readable storage medium of claim 72, wherein a sequencing-correction sequence in the plurality of sequencing-correction sequences comprises all or a portion of a reference sequence corresponding to a predefined category in a plurality of predefined categories.

74. The non-transitory computer-readable storage medium of claim 72 or 73, wherein the plurality of sequencing-correction sequences comprises all or a portion of a first target nucleotide sequence corresponding to the first predefined category.

75. The non-transitory computer-readable storage medium of any one of claims 71-74, wherein the sequencing correction factor is a fixed value.

76. The non-transitory computer-readable storage medium of any one of claims 64-75, further comprising correcting the amount of the first predefined category in the sample using an abundance correction factor.

77. The non-transitory computer-readable storage medium of claim 64, wherein the calculating the amount of the first predefined category represented in the sample is calculated as the product of (i) an abundance correction factor, (ii) an extraction correction factor, (iii) a sequencing correction factor, (iv) the known quantity of the internal control material, and (v) the first normalized read count divided by the second normalized read count.

78. The non-transitory computer-readable storage medium of any one of claims 64-77, wherein the determining the first read count and the second read count further comprises:
mapping the first plurality of sequence reads to all or a portion of a first reference sequence corresponding to the first predefined category; and
mapping the second plurality of sequence reads to all or a portion of a second reference sequence corresponding to the internal control material.

79. The non-transitory computer-readable storage medium of claim 78, further comprising:
determining a first count of the number of sequence reads, in the first plurality of sequence reads, that map to a first target nucleotide sequence obtained from the first reference sequence corresponding to the first predefined category;
determining a second count of the number of sequence reads, in the second plurality of sequence reads, that map to a second target nucleotide sequence obtained from the second reference sequence corresponding to the internal control material;
normalizing the first count based on the length of the first target nucleotide sequence; and
normalizing the second count based on the length of the second target nucleotide sequence, thereby obtaining the first normalized read count and the second normalized read count, respectively.

80. The non-transitory computer-readable storage medium of any one of claims 64-79, wherein the first normalized read count and the second normalized read count are expressed as reads per kilobase per million mapped reads (RPKM).

81. The non-transitory computer-readable storage medium of any one of claims 64-80, wherein the first target nucleotide sequence length is determined from all or a portion of a reference sequence corresponding to the first predefined category.

82. The non-transitory computer-readable storage medium of any one of claims 64-81, wherein the first target nucleotide sequence length is determined from at least two non-contiguous regions of a reference sequence corresponding to the first predefined category.

83. The non-transitory computer-readable storage medium of any one of claims 64-81, wherein the first target nucleotide sequence length is determined from a single contiguous region of a reference sequence corresponding to the first predefined category.

84. The non-transitory computer-readable storage medium of any one of claims 64-83, wherein the first target nucleotide sequence length comprises at least 50 base pairs.

85. The non-transitory computer-readable storage medium of any one of claims 64-84, wherein the first target nucleotide sequence length and the second target nucleotide sequence length are different.

86. The non-transitory computer-readable storage medium of any one of claims 64-85, wherein the second target nucleotide sequence length is determined from all or a portion of a reference sequence corresponding to the internal control material.

87. The non-transitory computer-readable storage medium of any one of claims 64-86, wherein the second target nucleotide sequence length is determined from at least two non-contiguous regions of a reference sequence corresponding to the internal control material.

88. The non-transitory computer-readable storage medium of any one of claims 64-87, wherein the second target nucleotide sequence length is determined from a single contiguous region of a reference sequence corresponding to the internal control material.

89. The non-transitory computer-readable storage medium of any one of claims 64-88, wherein the second target nucleotide sequence length comprises at least 50 base pairs.

90. The non-transitory computer-readable storage medium of any one of claims 64-89, wherein the sequencing reaction is a whole transcriptome sequencing reaction or a whole genome sequencing reaction.

91. The non-transitory computer-readable storage medium of any one of claims 64-90, wherein the sequencing dataset comprises at least 1×10³, at least 1×10⁴, at least 1×10⁵, at least 1×10⁶, at least 1×10⁷, at least 1×10⁸, or at least 2×10⁸ sequence reads.

92. The non-transitory computer-readable storage medium of any one of claims 64-91, wherein the first plurality of sequence reads collectively maps to at least 50 base pairs of a first reference sequence corresponding to the first predefined category.

93. The non-transitory computer-readable storage medium of any one of claims 64-92, wherein the second plurality of sequence reads collectively maps to at least 50 base pairs of a second reference sequence corresponding to the internal control material.

94. The non-transitory computer-readable storage medium of any one of claims 64-93, wherein the sample is obtained from a biological subject.

95. The non-transitory computer-readable storage medium of any one of claims 64-94, wherein the sample is obtained from a human with a disease condition.

96. The non-transitory computer-readable storage medium of any one of claims 64-95, wherein the first predefined category is a microorganism.

97. The non-transitory computer-readable storage medium of claim 96, wherein the microorganism is selected from the group consisting of bacterial, fungal, viral, and parasitic.

98. The non-transitory computer-readable storage medium of claim 96 or 97, wherein the microorganism is a pathogen.

99. The non-transitory computer-readable storage medium of any one of claims 64-98, wherein the source other than the first predefined category is human.

100. The non-transitory computer-readable storage medium of any one of claims 64-99, wherein the sequencing dataset further includes a third plurality of sequence reads, wherein each respective sequence read in the third plurality of sequence reads is determined by a sequencing of a nucleic acid molecule in the one or more nucleic acid molecules originating from the source other than the first predefined category.

101. The non-transitory computer-readable storage medium of claim 100, further comprising:
mapping the third plurality of sequence reads to all or a portion of a third reference sequence corresponding to the source other than the first predefined category;
determining a third count of the number of sequence reads, in the third plurality of sequence reads, that map to a third target nucleotide sequence obtained from the third reference sequence corresponding to the source other than the first predefined category;
normalizing the third count based on the length of the third target nucleotide sequence, thereby determining a third normalized read count for the number of sequence reads originating from the source other than the first predefined category; and
calculating the amount of the first predefined category in the sample based at least in part on the third normalized read count.

102. The non-transitory computer-readable storage medium of claim 101, wherein the third normalized read count is expressed as reads per kilobase per million mapped reads (RPKM).

103. The non-transitory computer-readable storage medium of claim 101 or 102, wherein the third target nucleotide sequence length is determined from at least two non-contiguous regions of the third reference sequence corresponding to the source other than the first predefined category.

104. The non-transitory computer-readable storage medium of claim 101 or 102, wherein the third target nucleotide sequence length is determined from a single contiguous region of the third reference sequence corresponding to the source other than the first predefined category.

105. The non-transitory computer-readable storage medium of any one of claims 100-104, wherein the third plurality of sequence reads collectively maps to at least 50 base pairs of a third reference sequence corresponding to the source other than the first predefined category.

106. The non-transitory computer-readable storage medium of any one of claims 64-105, wherein:
the first predefined category is in a plurality of predefined categories in the sample and the dataset comprises a corresponding plurality of sequence reads, for each predefined category in the plurality of predefined categories, including the first plurality of sequence reads for the first predefined category, and
the method further comprises, for each respective predefined category beyond the first predefined category in the plurality of predefined categories:
determining a respective normalized read count for the number of sequence reads originating from the respective predefined category, wherein the respective normalized read count is normalized based on a corresponding target nucleotide sequence length for the respective predefined category, and
calculating the amount of the respective predefined category in the sample based on the respective normalized read count for the number of sequence reads originating from the respective predefined category, the second normalized read count, and the known quantity of the internal control material.

107. The non-transitory computer-readable storage medium of claim 106, further comprising:
mapping, for each respective predefined category beyond the first predefined category in the plurality of predefined categories, the corresponding plurality of sequence reads to all or a portion of a reference sequence corresponding to the respective predefined category;
determining a count of the number of sequence reads, in the corresponding plurality of sequence reads, that map to a target nucleotide sequence obtained from the corresponding reference sequence;
normalizing the count based on the length of the target nucleotide sequence, thereby determining the respective normalized read count for the number of sequence reads originating from the respective predefined category; and
calculating the amount of the respective predefined category in the sample based on the respective normalized read count, the second normalized read count, and the known quantity of the internal control material.

108. The non-transitory computer-readable storage medium of claim 107, wherein the calculating the amount of the respective predefined category in the sample is determined by dividing the product of (i) the known quantity of the internal control material and (ii) the respective normalized read count for the number of sequence reads originating from the respective predefined category by the second normalized read count for the number of sequence reads originating from the internal control material.

109. The non-transitory computer-readable storage medium of claim 107, wherein the calculating the amount of the respective predefined category in the sample is determined by dividing the product of (i) an abundance correction factor, (ii) an extraction correction factor, (iii) a sequencing correction factor, (iv) the known quantity of the internal control material, and (v) the respective normalized read count for the number of sequence reads originating from the respective predefined category by the second normalized read count for the number of sequence reads originating from the internal control material.

110. The non-transitory computer-readable storage medium of any one of claims 107-109, wherein the respective normalized read count is expressed as reads per kilobase per million mapped reads (RPKM).

111. The non-transitory computer-readable storage medium of any one of claims 107-109, wherein the respective target nucleotide sequence length is determined from at least two non-contiguous regions of the reference sequence corresponding to the respective predefined category.

112. The non-transitory computer-readable storage medium of any one of claims 107-109, wherein the respective target nucleotide sequence length is determined from a single contiguous region of the reference sequence corresponding to the respective predefined category.

113. The non-transitory computer-readable storage medium of any one of claims 107-112, wherein the respective target nucleotide sequence length comprises at least 50 base pairs.

114. The non-transitory computer-readable storage medium of any one of claims 107-113, wherein the first target nucleotide sequence length for the first predefined category and the respective target nucleotide sequence length, for the respective predefined category beyond the first predefined category in the plurality of predefined categories, are different.

115. The non-transitory computer-readable storage medium of any one of claims 106-114, wherein the respective predefined category is a microorganism.

116. The non-transitory computer-readable storage medium of claim 115, wherein the microorganism is selected from the group consisting of bacterial, fungal, viral, and parasitic.

117. The non-transitory computer-readable storage medium of claim 115 or 116, wherein the microorganism is a pathogen.

118. The non-transitory computer-readable storage medium of any one of claims 106-55, wherein the respective plurality of sequence reads collectively maps to at least 50 base pairs of a reference sequence corresponding to the respective predefined category.

119. The non-transitory computer-readable storage medium of any one of claims 106-118, wherein the amount of the first predefined category in the sample and the amount of the respective predefined category other than the first predefined category in the plurality of predefined categories, in the sample are different.

120. The non-transitory computer-readable storage medium of any one of claims 64-119, further comprising generating a report including the amount of the first predefined category in the sample.

121. The non-transitory computer-readable storage medium of claim 120, wherein the report comprises a first therapeutic regimen based on the amount of the first predefined category.

122. The non-transitory computer-readable storage medium of claim 121, wherein the report comprises an antimicrobial resistance status for the first predefined category, and the first therapeutic regimen is based on the amount of the first predefined category and the antimicrobial resistance status for the first predefined category.

123. The non-transitory computer-readable storage medium of any one of claims 120-122, wherein the report comprises a patient status.

124. The non-transitory computer-readable storage medium of any one of claims 120-123, wherein the first predefined category is in a plurality of predefined categories in the sample, and the report further comprises, for each respective predefined category beyond the first predefined category in the plurality of predefined categories, an amount of the respective predefined category in the sample, calculated based on a respective normalized read count for the respective predefined category, the second normalized read count for the internal control material, and the known quantity of the internal control material.

125. The non-transitory computer-readable storage medium of any one of claims 102-124, wherein the generating a report comprises transmitting the report to a cloud computing infrastructure.

126. A computer system for determining an amount of a first predefined category represented in a sample, the computer system comprising:
a processor; and
a memory addressable by the processor, the memory storing at least one program for execution by the at least one processor, the at least one program comprising instructions for:
obtaining, in electronic form, a sequencing dataset comprising a first plurality of sequence reads and a second plurality of sequence reads originating from a sequencing of the sample, wherein the sample comprises (i) a plurality of nucleic acid molecules originating from the first predefined category, (ii) a plurality of nucleic acid molecules originating from a source other than the first predefined category, and (iii) a known quantity of an internal control material comprising one or more nucleic acid molecules, wherein:
each respective sequence read in the first plurality of sequence reads is determined by a sequencing of a nucleic acid molecule in the plurality of nucleic acid molecules originating from the first predefined category, and
each respective sequence read in the second plurality of sequence reads is determined by a sequencing of a nucleic acid molecule in the plurality of nucleic acid molecules originating from the internal control material;
determining, from the first plurality of sequence reads, a first normalized read count for the number of sequence reads originating from the first predefined category, wherein the first normalized read count is normalized based on a first target nucleotide sequence length;
determining, from the second plurality of sequence reads, a second normalized read count for the number of sequence reads originating from the internal control material, wherein the second normalized read count is normalized based on a second target nucleotide sequence length; and
calculating the amount of the first predefined category in the sample based on the first normalized read count, the second normalized read count, and the known quantity of the internal control material.

127. The method of any one of claims 1-63, wherein the one or more nucleic acid molecules originating from the first predetermined category or the second predetermined category comprises 2, 3, 4, 5, 6, 7, 8, 9, 10, 11, 12, 13, 14, 15, 16, 17, 18, 19, or 20 or more nucleic acid molecules.

128. The method of any one of claims 1-63, wherein the one or more nucleic acid molecules originating from the first predetermined category or the second predetermined category comprises 30, 40, 50, 60, 70, 80, 90, 100, 110, 120, 130, 140, 150, 160, 170, 180, 190, or 200 or more nucleic acid molecules.

129. The method of any one of claims 1-63, wherein the one or more nucleic acid molecules originating from the first predetermined category or the second predetermined category comprises 200, 300, 400, 500, 600, 700, 800, 900, 1000, 1100, 1200, 1300, 1400, 1500, 1600, 1700, 1800, 1900, or 2000 or more nucleic acid molecules.

130. The method of any one of claims 1-631, wherein the one or more nucleic acid molecules originating from the first predetermined category or the second predetermined category comprises 3000, 4000, 5000, 6000, 7000, 8000, 9000, 10000, 11000, 12000, 13000, 14000, 15000, 16000, 17000, 18000, 19000, or 20000 or more nucleic acid molecules.

131. The method of any one of claims 1-63, wherein the one or more nucleic acid molecules originating from the first predetermined category or the second predetermined category comprises 30000, 40000, 50000, 60000, 70000, 80000, 90000, 100000, 110000, 120000, 130000, 140000, 150000, 160000, 170000, 180000, 190000, or 200000 or more nucleic acid molecules.

132. The method of any one of claims 1-63 or claims 127-131, wherein the first plurality of sequence reads, the second plurality of sequence reads, or the combination of the first plurality of sequence reads and the second plurality of sequence reads comprises 200, 300, 400, 500, 600, 700, 800, 900, 1000, 1100, 1200, 1300, 1400, 1500, 1600, 1700, 1800, 1900, or 2000 or more sequence reads.

133. The method of any one of claims 1-63 or claims 127-131, wherein the first plurality of sequence reads, the second plurality of sequence reads, or the combination of the first plurality of sequence reads and the second plurality of sequence reads comprises 1000, 3000, 4000, 5000, 6000, 7000, 8000, 9000, 10000, 11000, 12000, 13000, 14000, 15000, 16000, 17000, 18000, 19000, or 20000 or more sequence reads.

134. The method of any one of claims 1-63 or claims 127-131, wherein the first plurality of sequence reads, the second plurality of sequence reads, or the combination of the first plurality of sequence reads and the second plurality of sequence reads comprises 30000, 40000, 50000, 60000, 70000, 80000, 90000, 100000, 110000, 120000, 130000, 140000, 150000, 160000, 170000, 180000, 190000, or 200000 or more sequence reads.

135. The method of any one of claims 1-63 or claims 127-131, wherein the first plurality of sequence reads, the second plurality of sequence reads, or the combination of the first plurality of sequence reads and the second plurality of sequence reads comprises 1 million sequence reads, 2 million sequence reads, five million sequence reads, ten million sequence reads or twenty million sequence reads.

136. The method of any one of claims 1-63 or claims 127-131, wherein the first plurality of sequence reads, the second plurality of sequence reads, or the combination of the first plurality of sequence reads and the second plurality of sequence reads consists of between 1 million sequence reads and 25 million sequence reads, between 2 million sequence reads and 24 million sequence reads, between five million sequence reads and 23 million sequence reads, or between ten million sequence reads and twenty million sequence reads.

137. The method of any one of claims 1-63 or claims 127-131, wherein the first plurality of sequence reads, the second plurality of sequence reads, or the combination of the first plurality of sequence reads and the second plurality of sequence reads consists of between 500 sequence reads and 10,000 sequence reads, 800 sequence reads 5,000 sequence reads, between 600 sequence reads and 4,000 sequence reads, or between 800 sequence reads and twenty-five million sequence reads.

138. The method of any one of claims 132 through 137, wherein the first plurality of sequence reads, the second plurality of sequence reads, or the combination of the first plurality of sequence reads and the second plurality of sequence reads have an average sequence length of between 50 nucleotides and 500 nucleotides.

139. The method of any one of claims 132 through 137, wherein the first plurality of sequence reads, the second plurality of sequence reads, or the combination of the first plurality of sequence reads and the second plurality of sequence reads have an average sequence length of between 50 nucleotides and 150 nucleotides.

140. The method of any one of claims 132 through 137, wherein the first plurality of sequence reads, the second plurality of sequence reads, or the combination of the first plurality of sequence reads and the second plurality of sequence reads have an average sequence length of between 50 nucleotides and 10000 nucleotides or an average sequence length of between 3000 nucleotides and 10000 nucleotides.

141. The method of claim 15, wherein the first reference sequence or the second reference sequence comprises 1000 nucleotides, 2000 nucleotides, 10,000 nucleotides, 100,000 nucleotides, 1×10⁶ nucleotides, or 1×10⁷ nucleotides.

142. The method of claim 30, wherein the first reference sequence comprises 1000 nucleotides, 2000 nucleotides, 10,000 nucleotides, 100,000 nucleotides, 1×10⁶ nucleotides, or 1×10⁷ nucleotides.

143. The method of claim 30, wherein the second reference sequence comprises 1000 nucleotides, 2000 nucleotides, 10,000 nucleotides, 100,000 nucleotides, 1×10⁶ nucleotides, or 1×10⁷ nucleotides.
